(19) United States
(12) Patent Application Publication
(10) Pub. No.: US 2018/0011102 A1
(43) Pub. Date: Jan. 11, 2018

(54) THE PROTEIN KINASE ACTIVITY OF PHOSPHOGLYCERATE KINASE 1 AS A TARGET FOR CANCER TREATMENT AND DIAGNOSIS

(71) Applicant: Board of Regents, The University of Texas System, Austin, TX (US)

(72) Inventor: Zhimin LU, Pearland, TX (US)

(21) Appl. No.: 15/541,501

(22) PCT Filed: Jan. 5, 2016
(86) PCT No.: PCT/US2016/012153

§ 371 Date: Jul. 5, 2017

Related U.S. Application Data
(60) Provisional application No. 62/099,899, filed on Jan. 5, 2015.

(51) Int. Cl. G01N 33/574; A61K 45/06; A61K 31/713; G01N 33/50

(52) U.S. Cl. CPC G01N 33/57484; G01N 33/5011; A61K 45/06; C12Y 207/02003; A61K 31/713; G01N 2333/91225; G01N 2440/14; G01N 2800/52

(57) ABSTRACT

Compositions and methods for characterizing cancer cells by determining a marker of PGK1 activity. For example, methods are provided for predicting a patient response to a PGK1 inhibitor, a MEK/ERK inhibitor, an EGFR inhibitor, or a PIN1 inhibitor therapy. Methods for treating patients with such therapies are likewise provided.

The present application claims the priority benefit of U.S. provisional application No. 62/099,899, filed Jan. 5, 2015, the entire contents of which is incorporated herein by reference.

The invention was made with government support under Grant Nos. RO1 CA109035 and RO1 CA169603 awarded by the National Institutes of Health. The government has certain rights in the invention.

### BACKGROUND OF THE INVENTION

### 1. Field of the Invention

The present invention relates generally to the field of molecular biology, biochemistry, oncology and medicine. More particularly, it concerns methods and composition for characterizing cancer cells.

### 2. Description of Related Art

Most cancer cells even in the presence of ample oxygen predominantly produce energy by a high rate of glycolysis followed by lactic acid fermentation in the cytosol, rather than by oxidation of pyruvate in mitochondria as in most normal cells. This tumor-specific Warburg effect promotes tumor progression (Koppenol et al., 2011; Vander Heiden et al., 2009). Mitochondrial oxidative phosphorylation is regulated by the availability of oxygen and pyruvate, which are the terminal electron acceptor and the primary carbon source, respectively, for this process (Brown, 1992). Mitochondrial pyruvate metabolism is regulated by pyruvate dehydrogenase kinase (PDHK or PDK), which has four isoforms (PDHK1-4), and pyruvate dehydrogenase (PDH) (Roche and Hiromasa, 2007). PDHK1, whose expression is upregulated by hypoxia-inducible factor 1α (HIF1α), phosphorylates S293 of the PDH E1α subunit and inactivates the PDH complex that normally converts pyruvate to acetyl-coA and CO₂; this results in an inhibition of pyruvate metabolism and the tricarboxylic acid (TCA) cycle-coupled electron transport and thus attenuation of mitochondrial respiration and ROS production (Holness and Sugden, 2003; Kim et al., 2006; Papandreou et al., 2006). By excluding pyruvate from mitochondrial consumption, PDHK1 induction may promote glycolysis and increase the rate of conversion of pyruvate to lactate. Cancer cells also depend on various substrates other than glucose, such as glutamine, for mitochondrial metabolism (Gao et al., 2009). However, the mechanisms underlying coordinated regulation of glycolysis, TCA cycle, and glutaminolysis by oncogenes remain elusive.

Pyruvate kinase M2, the second ATP-generating enzyme in the glycolytic pathway, catalyzes the transfer of a phosphate group from phosphoenolpyruvate (PEP) to adenosine diphosphate for the production of pyruvate and adenosine triphosphate (ATP). PKM2 also acts as a protein kinase phosphorylating histone H3, Bub3, Stat3, and ERK (Yang et al., 2012; Gao et al., 2012; Jiang et al., 2014; Lowery et al., 2007). Phosphoglycerate kinase 1 (PGK1), the first ATP-generating enzyme in the glycolytic pathway, catalyzes the transfer of the high-energy phosphate from the 1-position of 1,3-diphosphoglycerate (1,3-BPG) to ADP, which leads to the generation of 3-phosphoglycerate (3-PG) and ATP (Marin-Hernandez et al., 2009; Semenza, 2010). PGK1 expression is upregulated in human breast cancer (Zhang et al., 2005), pancreatic ductal adenocarcinoma (Hwang et al., 2006), radioresistant astrocytomas (Yan et al., 2012), and multidrug-resistant ovarian cancer cells (Duan et al., 2002), as well as in metastatic gastric cancer, colon cancer, and hepatocellular carcinoma cells (Zieker et al., 2010; Ahmad et al., 2013; Ai et al., 2011). In spite of its overexpression in many types of human cancer, whether PGK1 has other functions besides its role in catalyzing a glycolytic reaction and the mechanisms underlying PGK1-promoted tumor development remain largely unclear.

### SUMMARY OF THE INVENTION

As taught herein, hypoxia, activation of EGFR, and expression of K-Ras G12V and B-Raf V600E induce ERK1/2 phosphorylation-dependent and PIN1 cis-trans isomerization-regulated mitochondrial translocation of PGK1. Mitochondrial PGK1, acting as a protein kinase, phosphorylates and activates PDHK1. Without being bound by theory, this phosphorylation inhibits mitochondrial pyruvate metabolism and ROS production and enhances glycolysis and glutaminolysis-driven lipid synthesis, thereby promoting tumor development.

In one embodiment, there is provided a composition for use in treating a patient having a cancer determined to comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level. For example, such a composition may comprise an effective amount of a PGK1 inhibitor, a MEK/ERK inhibitor, an EGFR inhibitor, a PIN1 inhibitor, or a combination thereof. In some aspects, the composition may comprise at least a second therapeutic.

In some aspects, the PGK1 inhibitor may be a small molecule PGK1 inhibitor. Such a small molecule inhibitor may selectively inhibit the kinase activity of PGK1. In some aspects, the PGK1 inhibitor may comprise an inhibitory polynucleotide complementary to all or part of a PGK1 gene. Such an inhibitory polynucleotide may be a siRNA.

In some aspects, the MEK-ERK inhibitor may be U0126, AZD6244, PD98059, GSK1120212, GDC-0973, RDEA119, PD18416, CI1040 or FR180204. In some aspects, the EGFR inhibitor may be AG1478.

In some embodiments, there is provided a method for treating a patient having a cancer comprising (a) selecting a patient whose cancer cells have been determined to comprise comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level; and (b) treating the patient with a PGK1 inhibitor therapy, a MEK/ERK inhibitor therapy, an EGFR inhibitor therapy, and/or a PIN1 inhibitor therapy. Thus, in a related embodiment, a composition comprising a PGK1 inhibitor therapy, a MEK/ERK inhibitor therapy, a EGFR inhibitor therapy, or a PIN1 inhibitor therapy is provided for use in treating a patient having a cancer determined to comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level.

In yet a further embodiment, a method for treating a patient having a cancer is provided comprising (a) selecting a patient whose cancer cells have been determined to comprise an elevated level of PGK1 S203 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of PGK1 Y324 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of PDHK1 T338 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of PDH S293 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of CDC45 S386 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of histone H3 S10 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of Beclin-1 S30 phosphorylation compared to a reference level; and (ii) treating the patient with a PGK1 inhibitor therapy, a MEK/ERK inhibitor therapy, an EGFR inhibitor therapy, and/or a PIN1 inhibitor therapy.

In yet a further embodiment, a method for treating a patient having a cancer is provided comprising (a) selecting a patient whose cancer cells have been determined to comprise an elevated level of mitochondrially-located PGK1 compared to a reference level; and (ii) treating the patient with a PGK1 inhibitor therapy, a MEK/ERK inhibitor therapy, an EGFR inhibitor therapy, and/or a PIN1 inhibitor therapy.

In some embodiments, there is provided a method of selecting a patient having a cancer for a PGK1 inhibitor therapy, a MEK/ERK inhibitor therapy, a EGFR inhibitor therapy, or a PIN1 inhibitor therapy comprising determining whether cancer cell of the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level, wherein if the patient comprises an elevated level then the patient is selected for a PGK1 inhibitor therapy, a MEK/ERK inhibitor therapy, an EGFR inhibitor therapy, or a PIN1 inhibitor therapy. Thus, in some aspects, a method is provided of selecting a patient having a cancer for a PGK1 inhibitor therapy, a MEK/ERK inhibitor therapy, an EGFR inhibitor therapy, or a PIN1 inhibitor therapy comprising: (a) determining whether cancer cells of the patient comprise an elevated level of any of 1, 2, 3, 4, 5, and/or 6; and (b) selecting a patient for a PGK1 inhibitor therapy, a MEK/ERK inhibitor therapy, an EGFR inhibitor therapy, or a PIN1 inhibitor therapy if cancer cells of the patient comprise an elevated level of any of 1, 2, 3, 4, 5, and/or 6.

In one embodiment, a method is provided for predicting a response to a PGK1 inhibitor therapy, a MEK/ERK inhibitor therapy, a EGFR inhibitor therapy, or a PIN1 inhibitor therapy in a patient having cancer comprising determining whether cancer cells of the patient comprise: (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level, wherein the patient is predicted to have a favorable response to a PGK1 inhibitor therapy, a MEK/ERK inhibitor therapy, an EGFR inhibitor therapy, or a PIN1 inhibitor therapy if cancer cells from the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level; or wherein the patient is not predicted to have a favorable response to a PGK1 inhibitor therapy, a MEK/ERK inhibitor therapy, an EGFR inhibitor therapy, or a PIN1 inhibitor therapy if cancer cells from the patient do not comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level. In some aspects, a method is provided for predicting a response to a PGK1 inhibitor therapy, a MEK/ERK inhibitor therapy, an EGFR inhibitor therapy, or a PIN1 inhibitor therapy in a patient having a cancer comprising (a) determining whether the cancer cells of the patient comprise an elevated level of any of 1, 2, 3, 4, 5, and/or 6 compared to a reference level; and (b) identifying the patient as predicted to have a favorable response to a PGK1 inhibitor therapy, a MEK/ERK inhibitor therapy, an EGFR inhibitor therapy, or a PIN1 inhibitor therapy if cancer cells from the patient comprise an elevated level of any one of 1, 2, 3, 4, 5, and/or 6; or identifying the patient as not predicted to have a favorable response to a PGK1 inhibitor therapy, a MEK/ERK inhibitor therapy, a EGFR inhibitor therapy, or a PIN1 inhibitor therapy if cancer cells from the patient do not comprise an elevated level of any of 1, 2, 3, 4, 5, and/or 6.

As used in the context of methods of the embodiments a “favorable response” to a therapy, such as a PGK1 inhibitor therapy, a MEK/ERK inhibitor therapy, an EGFR inhibitor therapy, or a PIN1 inhibitor therapy, can comprise reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, increased patient survival and/or an increase in the sensitivity of the tumor to an anticancer therapy.

In some aspects, a method of predicting a response further comprises reporting whether cancer cells of the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level. In still further aspects, a method can comprise reporting whether a cancer is predicted to respond to a PGK1 inhibitor therapy, a MEK/ERK inhibitor therapy, an EGFR inhibitor therapy, or a PIN1 inhibitor therapy. In certain aspects, methods of the embodiments comprise reporting results, such as by providing a written, electronic or oral report. In some aspects, a report is provided to the patient. In still further aspects, the report is provided to a third party, such an insurance company or health care provider (e.g., a doctor or hospital).

In some embodiments, a method is provided for determining a prognosis in a patient having a cancer comprising determining whether cancer cells of the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level, wherein if the cancer cells comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation, then the patient is predicted to have an aggressive cancer. In some aspects, a method is provided for determining a prognosis in a patient having a cancer comprising determining whether cancer cells of the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation compared to a reference level, wherein if the cancer cells comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation, then the patient is predicted to have an aggressive cancer. In some aspects, a method is provided for determining a prognosis in a patient having a cancer comprising (a) determining whether cancer cells of the patient comprise an elevated level of any of 1, 2, 3, 4, 5, 6, 7 or 8 compared to a reference level; and (b) identifying the patient as predicted to have an aggressive cancer, if cancer cells from the patient comprise an elevated level of any of 1, 2, 3, 4, 5, 6, 7 or 8; or identifying the patient as not predicted to have an aggressive cancer, if cancer cells from the patient do not comprise an elevated level of any of 1, 2, 3, 4, 5, 6, 7 or 8.

In some embodiments, a method is provided for determining a prognosis in a patient having a cancer comprising determining whether cancer cells of the patient comprise (1) an elevated level of PGK1 S203 phosphorylation or (2) an elevated level of PDHK1 T338 phosphorylation compared to a reference level, wherein if the cancer cells comprise (1) an elevated level of PGK1 S203 phosphorylation or Y324 phosphorylation or (2) an elevated level of PDHK1 T338 phosphorylation, then the patient is predicted to have an aggressive cancer. In some aspects, a method is provided for determining a prognosis in a patient having a cancer comprising determining whether cancer cells of the patient comprise (1) an elevated level of PGK1 S203 phosphorylation or Y324 phosphorylation and (2) an elevated level of PDHK1 T338 phosphorylation compared to a reference level, wherein if the cancer cells comprise (1) an elevated level of PGK1 S203 phosphorylation or Y324 phosphorylation and (2) an elevated level of PDHK1 T338 phosphorylation, then the patient is predicted to have an aggressive cancer. Thus, in some aspects, a method is provided for determining a prognosis in a patient having a cancer comprising: (a) determining whether cancer cells of the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation compared to a reference level; and (b) identifying the patient as predicted to have an aggressive cancer, if cancer cells from the patient comprise the elevated level or identifying the patient as not predicted to have an aggressive cancer, if cancer cells from the patient do not comprise the elevated level.

In some aspects, a method of determining a prognosis can comprise determining the grade of cancer or the probability that the cancer will metastasize. In certain aspects, a method of determining a prognosis further comprises reporting whether cancer cells from the patient comprise (1) an elevated level of PGK1 S203 phosphorylation; (2) an elevated level of PGK1 Y324 phosphorylation; (3) an elevated level of PDHK1 T338 phosphorylation; (4) an elevated level of PDH S293 phosphorylation; (5) an elevated level of CDC45 S386 phosphorylation; (6) an elevated level of histone H3 S10 phosphorylation; or (7) an elevated level of Beclin-1 S30 phosphorylation. In still further aspects, a method can comprise reporting whether a cancer is an aggressive cancer or reporting a grade for the cancer. In certain aspects, methods of the embodiments comprise reporting results, such as by providing a written, electronic or oral report. In some aspects, a report is provided to the patient. In still further aspects, the report is provided to a third party, such an insurance company or health care provider (e.g., a doctor or hospital).

In some aspects, a method of determining a prognosis may further comprise administering one or more anticancer therapy to the patient if the patient is predicted to have an aggressive cancer. In some aspects, the reference level may be a level from a non-cancer cell or a level from an early stage or low grade cancer cell.

In still a further embodiment a method for screening candidate anti-cancer agents (e.g., small molecule agents) is provided comprising determining the binding of PGK1 to PDHK1 (or a fragment thereof) and/or the phosphorylation of PDHK1 by PGK1 in the presence or absence of an agent, wherein an agent that disrupts binding of PGK1 to PDHK1 (or a fragment thereof) and/or disrupts phosphorylation of PDHK1 by PGK1 is a candidate PGK1 inhibitor or anti-cancer agent. In yet still a further embodiment, there is provided a method for screening candidate PGK1 inhibitors or anti-cancer agents comprising (a) determining the binding of PGK1 to PDHK1 (or a fragment thereof) and/or the phosphorylation of PDHK1 by PGK1 in the presence or absence of an agent; and (b) selecting a candidate PGK1 inhibitor or anti-cancer agent based on the agent disrupting the binding of PGK1 to PDHK1 (or a fragment thereof) and/or the phosphorylation of PDHK1 by PGK1. In certain aspects, methods for screening of the embodiments can involve screening of small molecules, peptides and/or polypeptides (e.g., antibodies). In certain aspects, the screening methods can be in a cell-free system. In further aspects screening is performed in cells, such as cells comprised in an organism. Additional components can, in some cases, be included in the screening assay, such as without limitation, additional polypeptides, lipids, carbohydrates, ATP, buffers, chelating agents, etc.

In some embodiments, a method is provided for predicting the severity of a cancer in a patient comprising (a) determining a level of PGK1 activity, a level of PGK1 S203 phosphorylation, a level of PGK1 Y324 phosphorylation, or a level of PGK1 mitochondrial localization in a patient sample; and (b) predicting the severity of a cancer in the subject based on the level of PGK1 activity, a level of PGK1 S203 phosphorylation, a level of PGK1 Y324 phosphorylation, or a level of PGK1 mitochondrial localization, wherein an elevated level of PGK1 activity, PGK1 S203 phosphorylation, or PGK1 mitochondrial localization relative to a reference level indicates a more severe cancer. In certain aspects, determining a level of PGK1 activity may comprise determining a level of PDHK1 T338 phosphorylation.

Various aspects of the embodiments involve determining a level of β-catenin activity, a level of PGK1 S203 phosphorylation; a level of PGK1 Y324 phosphorylation; a level of PDHK1 T338 phosphorylation; a level of PDH S293 phosphorylation; a level of CDC45 S386 phosphorylation; a level of histone H3 S10 phosphorylation; a level of Beclin-1 S30 phosphorylation; a level of PGK1 mitochondrial localization; a level of PGK1 isomerization; and/or a level of PGK1 activation. In certain aspects, this determining can comprise performing an ELISA, an immunoassay, a radioimmunoassay (RIA), Immunohistochemistry, an immunoradiometric assay, a fluoroimmunoassay, a chemiluminescent assay, a bioluminescent assay, a gel electrophoresis, a Western blot analysis, a southern blot, flow cytometry, in situ hybridization, positron emission tomography (PET), single photon emission computed tomography (SPECT) imaging) or a microscopic assay. For example, in some cases, a phosphorylation specific antibody is used to determine a level of PGK1, PDHK1, PDH, CDC45, histone H3, or Beclin-1 phosphorylation. In some aspects, a level of phosphorylation is determined as a ratio of phosphorylated protein:unphosphorylated protein in the same sample. In some aspects, a method of the embodiments is defined as an in vitro method in other aspects a method may be performed in vivo (e.g., by in vivo imaging).

Some aspects of the embodiment involves determining a level of PGK1 S203 phosphorylation; a level of PGK1 Y324 phosphorylation; a level of PDHK1 T338 phosphorylation; a level of PDH S293 phosphorylation; a level of CDC45 S386 phosphorylation; a level of histone H3 S10 phosphorylation; a level of Beclin-1 S30 phosphorylation; a level of PGK1 mitochondrial localization; a level of PGK1 isomerization; and/or a level of PGK1 activation relative to a reference level. Such a reference level may be a level from a non-cancer cell (e.g., from a healthy patient) or a level from an early stage or low-grade cancer cell.

Some aspects of the embodiments involve a patient, such as a patient having a cancer. As used herein a patient can be human or non-human animal patient (e.g., a dog, cat, mouse, horse, etc). In certain aspects, the patient has a cancer, such as an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, neuroendocrine tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some aspects, the cancer is a glioma. In some aspects, the cancer is an oncogenic EGFR, an oncogenic K-Ras or oncogenic B-Raf positive cancer.

Some aspects of the embodiments concern patient samples, such as a tissue sample, a fluid sample (e.g., blood, urine or stool), or a tumor biopsy sample. Such a sample can be directly obtained from a patient or can be obtained by a third party.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-I. Hypoxia- and activation of EGFR, K-Ras, and B-Raf-Induced Mitochondrial Translocation of PGK1 is Mediated by ERK1/2-dependent PGK1 S203 Phosphorylation FIGS. 1B and D-I. Immunoblotting and immunoprecipitation analyses were carried out using antibodies against the indicated proteins. FIG. 1A U87 cells were stimulated with or without hypoxia for 6 h and stained with an anti-PGK1 antibody, MitoTracker, and DAPI. FIG. 1B. U87 and U251 cells were stimulated with hypoxia for 6 h. Proteins from mitochondrial outer membrane (OM), intermembrane space (IMS), inner membrane (IM), and matrix (Ma) were isolated. FIG. 1C. U87 cells were stimulated with or without hypoxia for 6 h. Electron microscopic immunogold analysis with anti-PGK1 antibody was performed. Arrows indicate representative staining of mitochondrial PGK1. Dashed circles indicate mitochondria. FIG. 1D. Mitochondria fractions and total cell lysates were prepared from U87 cells pretreated with SP600125 (25 μM), SB203580 (10 μM), or U0126 (20 μM) for 30 min before being treated with hypoxia for 6 h. Tubulin was used as a cytosolic protein marker. FIG. 1E. EGFR-overxpressed U87 (U87/EGFR) cells pretreated with U0126 (20 μM) for 30 min were stimulated with or without EGF (100 ng/ml) for 6 h. Mitochondria fractions and total cell lysates were prepared. FIG. 1F. BxPC-3 cells were stably transfected with or without vectors expressing V5-KRAS G12V and the indicated Flag-ERK2 proteins (left panel). CHL1 cells were stably transfected with or without vectors expressing V5-BRAF V600E and the indicated Flag-ERK2 proteins (right panel). Mitochondria fractions and total cell lysates were prepared. FIG. 1G. In vitro kinase assays were carried out by mixing purified active ERK2 with purified WT GST-PGK1 or GST-PGK1 S203A in the presence of [γ³²P]ATP. The reaction mixture was separated for autoradiography and immunoblotting analyses. FIG. 1H. U87 cells transfected with vectors expressing the indicated SFB-tagged PGK1 proteins were pretreated with or without U0126 (20 μM) for 30 min before hypoxic stimulation for 6 h. Streptavidin agarose beads were used to pull down SFB-tagged proteins. FIG. 1I. U87 cells transfected with vectors expressing the indicated V5-tagged PGK1 proteins were treated with or without hypoxia for 6 h. Mitochondrial fractions and total cell lysates were prepared.

FIGS. 2A-I. PIN1 Binds to and cis-trans Isomerizes Phosphorylated PGK1 for Mitochondrial Translocation of PGK1. FIGS. 2A-F and H-I. Immunoblotting and immunoprecipitation analyses were carried out using antibodies against the indicated proteins. FIG. 2A U87 cells were pretreated with or without U0126 (20 μM) for 30 min before hypoxic stimulation for 6 h. FIG. 2B. U87 cells were treated with or without hypoxic stimulation for 6 h. A GST pull-down assay with the indicted GST-proteins was performed. FIG. 2C. U87 cells expressing the indicated PGK1 proteins were treated with or without hypoxic stimulation for 6 h. A GST pull-down assay with GST-PIN1 proteins was performed. FIG. 2D. An in vitro protein kinase assay was performed by mixing purified recombinant PGK1 with or without purified active His-ERK2, which was followed by a GST pull-down assay with the indicated GST-proteins. FIG. 2E. A GST pull-down assay was performed by mixing GST-PIN1 and the indicated purified recombinant PGK1 proteins. FIG. 2F. U87 cells expressing the indicated SFB-tagged PGK1 proteins were treated with or without hypoxia for 6 h. Streptavidin agarose beads were used to pull down SFB-tagged proteins. FIG. 2G. cis-trans isomerization assays were performed by mixing synthesized phosphorylated or nonphosphorylated oligopeptides of PGK1 containing the S203P204 motif or an oligopeptide of PGK1 containing the D203P204 motif with purified wild-type GST-PIN1 or GST-PIN1 C113A mutant. Data represent the means±SD of three independent experiments. FIG. 2H. PIN1^{-/-} cells were reconstituted to express the indicated PIN1 proteins (left panel). The total cell lysates and mitochondrial fractions were prepared from the indicated cells treated with or without hypoxia for 6 h (right panel). FIG. 2I V5-PGK1 S203D was expressed in PIN1^{+/+} cells and PIN1^{-/-} cells with or without reconstituted WT PIN1 or PIN1 C113A. Total cell lysates and mitochondrial fractions of the cells were prepared.

FIGS. 3A-H. PIN1 Regulates Binding of PGK1 to the TOM Complex. FIGS. 3A-G. Immunoblotting and immunoprecipitation analyses were carried out using antibodies against the indicated proteins. FIG. 3A. U87 cells were treated with or without hypoxia for 6 h. Total cell lysates were prepared. FIG. 3B. PIN1^{+/+} and the indicated PIN1^{-/-} cells were treated with hypoxia for 6 h. FIG. 3C. A GST pull-down assay was performed by mixing purified recombinant GST-PGK1 WT or GST-PGK1 S203D with His-TOM20 in the presence or absence of purified His-PIN1. FIGS. 3D-E. U87 cells expressing the indicated SFB-tagged PGK1 proteins were treated with or without hypoxia for 6 h. Streptavidin agarose beads were used to pull down SFB-tagged proteins. FIGS. 3F-G. U87 cells expressing the indicated V5-tagged PGK1 proteins were treated with or without hypoxia for 6 h. Total cell lysate, cytosolic, and mitochondrial fractions were prepared. FIG. 3H. U87 cells expressing the indicated V5-PGK1 proteins were stimulated with or without hypoxia for 6 h and stained with an anti-V5 antibody, MitoTracker, and DAPI.

FIGS. 4A-F. Mitochondrial PGK1 Phosphorylates PDHK1. FIGS. 4A-C and E-G. Immunoblotting analyses were performed with the indicated antibodies. FIG. 4A. U87 cells with or without PGK1 shRNA and with or without reconstituted expression of WT rPGK1 or rPGK1 S203A were stimulated with or without hypoxia for 6 h. Mitochondrial fractions of the cells were prepared and activity of PDH complex-mediated conversion of ¹⁴C-labeled pyruvate into ¹⁴C-labeled CO₂ was measured. Data represent the means±SD of three independent experiments. *p<0.01. FIG. 4B. U87 cells were stimulated with or without hypoxia for 6 h. Mitochondrial fractions of these cells were prepared. Immunoprecipitation analyses with an anti-PGK1 antibody were performed. FIG. 4C. GST pull-down analyses were performed by mixing bacterially purified SUMO-PDHK1 proteins with purified immobilized GST or GST-PGK1 on glutathione agarose beads. FIG. 4D. In vitro phosphorylation analyses were performed by mixing bacterially purified His-PGK1 and SUMO-PDHK1 in the presence of ATP. The mass spectrometry results of a fragment spectrum of a peptide at m/z 756.346 (mass error, ±4.2 ppm) matched to the doubly charged peptide 331-LFNYMYp(ST)APRPR-343 (SEQ ID NO: 10), suggesting that S337 or T338 was phosphorylated. The Mascot score was 49, Expectation Value: 4.7E-004; the SEQUEST score for this match was X_{corr}=3.5. FIG. 4E. In vitro phosphorylation analyses with autoradiography were performed by mixing purified WT PGK1 or PGK1 T378P with purified WT PDHK1 or PDHK1 T338A in the presence of [γ³²P]ATP. FIG. 4F. U251 and U87 cells with or without PGK1 shRNA and with or without reconstituted expression of WT rPGK1 or rPGK1 S203A were stimulated with or without hypoxia for 6 h.

FIGS. 5A-E. PDHK1 Phosphorylation by PGK1 Activates PDHK1. Immunoblotting analyses were performed with the indicated antibodies. FIG. 5A. Bacterially purified His-PDH with purified WT PDHK1 or PDHK1 T338A was mixed with purified WT PGK1 or PGK1 T378P. In vitro phosphorylation analyses were performed. FIG. 5B. U87 and U251 cells expressing PGK1 shRNA with or without reconstituted expression of WT rPGK1 or rPGK1 S203A were stimulated with or without hypoxia for 6 h. FIG. 5C. U87 and U251 cells expressing PDHK1 shRNA with or without reconstituted expression of WT rPDHK1 or rPDHK1 T338A were stimulated with or without hypoxia for 6 h. FIG. 5D. U87/EGFR cells expressing PGK1 shRNA and with or without reconstituted expression of WT rPGK1 or rPGK1 S203A were stimulated with or without EGF (100 ng/ml) for 6 h. FIG. 5E. BxPC-3 cells were stably transfected with or without vectors expressing V5-KRAS G12V and the indicated Flag-ERK2 proteins (left panel). CHL1 cells were stably transfected with or without vectors expressing V5-BRAF V600E and the indicated Flag-ERK2 proteins (right panel).

FIGS. 6A-G. PGK1-Mediated PDHK1 Phosphorylation Inhibits Mitochondrial Pyruvate. Metabolism, Induces Hypoxia-Induced ROS Production, and Promotes Glycolysis. FIGS. 6A-E. Data represent the means±SD of three independent experiments. *p<0.01, #p <0.05. FIG. 6A. U87 cells expressing PDHK1 shRNA with or without reconstituted expression of WT rPDHK1 or rPDHK1 T338A were stimulated with or without hypoxia for 6 h. Mitochondrial fractions of the cells were prepared and activity of PDH complex-mediated conversion of ¹⁴C-labeled pyruvate into ¹⁴C-labeled CO₂ was measured. FIG. 6B. U87 cells expressing PDHK1 shRNA with or without reconstituted expression of WT rPDHK1 or rPDHK1 T338A were stimulated with or without hypoxia for 6 h. Levels of mitochondrial acetyl-CoA were measured. FIG. 6C. U87 cells expressing PDHK1 shRNA with or without reconstituted expression of their WT counterparts and the indicated mutants were stimulated with or without hypoxia for 24 h. Levels of mitochondrial ROS were measured. FIG. 6D. U87 cells expressing PGK1 shRNA (left panel) or PDHK1 shRNA (right panel) with or without reconstituted expression of their WT counterparts and the indicated mutants were stimulated with or without hypoxia for 6 h. Levels of cytosolic pyruvate level were measured. FIG. 6E. U87 cells expressing PGK1 shRNA (left panel) or PDHK1 shRNA (right panel) with or without reconstituted expression of their WT counterparts and the indicated mutants were cultured in no-serum DMEM during hypoxia for 6 h. The media were collected for analysis of lactate production. FIG. 6F. U87/EGFR cells stimulated with or without EGF (100 ng/ml) for 24 h were labeled with D-[6-¹⁴C]-glucose or L-[U-¹⁴C]-glutamine for 2 h. The lipid synthesis of the cells was examined. FIG. 6G. Endogenous PGK1-depleted U87 cells with reconstituted expression of WT rPGK1 or rPGK1 S203D was labeled with L-[U-¹⁴C]-glutamine for 2 h. The lipid synthesis of the cells was examined.

FIGS. 7A-D. Mitrochondrial PGK1-Dependent PDHK1 Phosphorylation Promotes Cell Proliferation and Brain Tumorigenesis and Indicates a Poor Prognosis in GBM Patients. FIGS. 7A-B. The data are presented as the means±SD from three independent experiments. FIG. 7A. A total of 2×10⁵ U87 cells with or without PGK1 shRNA or PDHK1 shRNA expression and with or without reconstituted expression of their WT counterparts and the indicated mutants were plated for 4 days under hypoxic condition. The cells were then collected and counted. FIG. 7B. A total of 1×10⁶ U87 cells with or without PGK1 shRNA or PDHK1 shRNA expression and with or without reconstituted expression of their WT counterparts and the indicated mutants were intracranially injected into athymic nude mice (n=7 mice per group). After 28 days, the mice were sacrificed and examined for tumor growth. H&E-stained coronal brain sections show representative tumor xenografts. Tumor volume was calculated using length a and width b: V=ab²/2. FIG. 7C. IHC staining with anti-phospho-PGK1 S203, anti-phospho-PDHK1 T338, and anti-phospho-PDH S293 antibodies was performed on 50 human primary GBM specimens. Representative photos of four tumors are shown. FIG. 7D. The survival time for 50 patients with low (1-4 staining scores, top curve) versus high (4.1-8 staining scores, bottom curve) phosphorylation levels of PGK1 S203 (low, 14 patients; high, 36 patients) and PDHK1 T338 (low, 16 patients; high, 34 patients) were compared. The table shows the multivariate analysis results after adjustment for patient age, indicating the significance level of the association of PGK1 S203 (p=0.016) and PDHK1 T338 (p=0.017) phosphorylation with patient survival. Empty circles represent censored data from patients alive at last clinical follow-up.

FIG. 8. A Mechanism of Mitochondrial PGK1-Coordinated Glycolysis and TCA Cycle in Tumorigenesis. Hypoxia or activation of EGFR, K-Ras, and B-Raf induces ERK phosphorylation and PIN1 cis-trans isomerization-dependent translocation of PGK1 into mitochondria, where PGK1 phosphorylates PDHK1 at T338, leading to enhanced PDH S293 phosphorylation by PDHK1 and subsequently the suppression of PDH-dependent mitochondrial pyruvate metabolism and ROS production and the increase of glycolysis. This metabolic alteration promotes cell proliferation and tumorigenesis. Broken arrows: inhibited directions or reactions.

FIGS. 9A-E. Hypoxia Induces Mitochondrial Translocation of PGK1. FIG. 9A. Total cell lysate, cytosolic, and mitochondrial fractions were prepared from U87 and U251 cells stimulated with or without hypoxia for 6 h. Immunoblotting analyses were performed with the indicated antibodies. Hypoxia-induced HIF1α expression (left panel) was a control for hypoxic stimulation. WCL: whole-cell lysate; Cyto: cytosol; Mito: mitochondria. WB, Western blot. FIG. 9B. Cytosolic and mitochondrial fractions were prepared from U87 and U251 cells stimulated with hypoxia for 6 h. Immunoblotting analyses of equal percentages of cytosolic and mitochondrial fraction were performed with the indicated antibodies. WCL: whole-cell lysate; Cyto: cytosol; Mito: mitochondria. Immunoblotting analyses were performed with the indicated antibodies. The images were quantified by scanning densitometry. FIG. 9C. U87 cells transfected with HIF1α siRNA or a scrambled siRNA before hypoxia stimulation for 6 h. Mitochondrial fractions were prepared. Immunoblotting analyses were performed with the indicated antibodies. FIG. 9D. Isolated mitochondria from U87 or U251 cells stimulated with or without hypoxia for 6 h were treated with proteinase K in the presence or absence of Triton X-100 followed by immunoblotting analyses with the indicated antibodies. FIG. 9E. Isolated mitochondria from U87 or U251 cells stimulated with or without hypoxia for 6 h were treated with proteinase K in the presence or absence of digitonin (100 μM) followed by immunoblotting analyses with the indicated antibodies. TIMM22, a mitochondrial inner membrane protein, was a control.

FIGS. 10A-I. Mitochondrial Translocation of PGK1 is depended on ERK1/2-mediated PGK1 Phosphorylation. FIG. 10A. Total cell lysates were prepared from U87 cells pretreated with SP600125 (25 μM), SB203580 (10 μM), or U0126 (20 μM) for 30 min before being treated with hypoxia for 6 h. Immunoblotting analyses were performed with the indicated antibodies. FIG. 10B. Mitochondria fractions were prepared from U251 cells pretreated with U0126 (20 μM) for 30 min before being treated with hypoxia for 6 h. Immunoblotting analyses were performed with the indicated antibodies. FIG. 10C. U87 cells were pretreated with or without U0126 (20 μM) for 30 min before being treated with hypoxia for 6 h. Immunofluorescence analyses were carried out using an anti-PGK1 antibody, MitoTracker, and DAPI. FIG. 10D. U87 cells stably transfected with a vector or Flag-ERK2 K52R were treated with or without hypoxia for 6 h. Mitochondria fractions and total cell lysates were prepared. FIG. 10E. U87 cells were stably transfected with or without vectors expressing HA-MEK1 Q56P with the indicated Flag-ERK2 proteins. Mitochondria fractions and total cell lysates were prepared. Immunoblotting analyses were performed with the indicated antibodies. FIG. 10F. U87 cells were treated with or without hypoxia for 6 h. Immunoblotting and immunoprecipitation analyses were carried out using antibodies against the indicated proteins. FIG. 10G. Immobilized GST or GST-PGK1 protein was incubated with purified recombinant His-ERK2. Immunoblotting analyses were performed with the indicated antibodies. FIG. 10H. U87 cells expressing the indicated Flag-tagged ERK2 proteins were treated with or without hypoxia for 6 h. Immunoblotting analyses were performed with the indicated antibodies. FIG. 10I. U87 cells transfected with the vectors expressing the indicated SFB-tagged PGK1 proteins were treated with or without hypoxia for 6 h. Streptavidin agarose beads were used to pull down SFB-tagged proteins. Immunoblotting analyses were performed with the indicated antibodies.

FIGS. 11A-J. ERK1/2-mediated PGK1 S203 Phosphorylation Is Required for Mitochondrial Translocation of PGK1. Immunofluorescence, immunoprecipitation, and immunoblotting analyses were performed with the indicated antibodies. FIG. 11A. U87 cells stimulated with or without hypoxia for 6 h were stained with the indicated antibody, MitoTracker, and DAPI. FIG. 11B. U87 cells transfected with the vectors expressing the indicated SFB-tagged PGK1 proteins were pretreated with or without U0126 (20 μM) or SP600125 (25 μM) for 30 min before hypoxic stimulation for 6 h. Streptavidin agarose beads were used to pull down SFB-tagged proteins. FIG. 11C. U87 cells were stably transfected with or without vectors expressing HA-MEK1 Q56P with the indicated Flag-ERK2 proteins. Total cell lysates were prepared. FIG. 11D. U87 cells expressing the indicated PGK1 proteins were stimulated with or without hypoxia for 6 h and stained with an anti-V5 antibody, MitoTracker, and DAPI. FIG. 11E. Anti-PGK1 pS203 antibody was used to deplete the phosphorylated PGK1 from total cell lysates prepared from U87 cells treated with or without hypoxia for 6 h. The images were quantified by scanning densitometry. FIGS. 11F-G. Total cell lysates were prepared from U87 cells pretreated with AG1478 (100 nM) or DMSO for 30 min before being treated with hypoxia for 6 h. FIG. 11H. U87/EGFR cells stably expressing the indicated SFB-tagged PGK1 proteins were stimulated with or without EGF (100 ng/ml) for 6 h. Streptavidin agarose beads were used to pull down SFB-tagged proteins. FIG. 11I. U87/EGFR cells stably expressing the indicated V5-tagged PGK1 proteins were stimulated with or without EGF (100 ng/ml) for 6 h. Mitochondria fractions and total cell lysates were prepared. FIG. 11J. BxPC-3 cells were stably transfected with or without the vectors expressing V5-KRAS G12V and the indicated Flag-ERK2 proteins (upper panel). CHL1 cells were stably transfected with or without the vectors expressing V5-BRAF V600E and the indicated Flag-ERK2 proteins (bottom panel).

FIGS. 12A-C. PIN1 Binds to Phosphorylated PGK1. FIG. 12A. The molecular modeling analysis of PGK1 (Protein Data Bank ID: 2zgv) and PIN1 (Protein Data Bank ID: 3tcz) protein was performed using an online ZDOCK server (on the world wide web at zdock.umassmed.edu/). S203 of PGK1 is represented with yellow spheres. K63 and R69 of PIN1 are represented with red ribbons. FIG. 12B. Immobilized His-tagged synthesized phosphorylated or nonphosphorylated oligopeptide of PGK1 containing the S203P204 motif or oligopeptide of PGK1 containing the D203P204 motif was incubated with purified wild-type GST-PIN1. Immunoblotting analyses were performed with the indicated antibodies. FIG. 12C. Cytosolic and mitochondrial fractions were prepared from U87 cells transfected with vectors expressing V5-tagged PGK1 S203D proteins. Immunoblotting analyses of equal percentages of total cell lysate, cytosolic fraction, and mitochondrial fraction were performed with the indicated antibodies. WCL: whole-cell lysate; Cyto: cytosol; Mito: mitochondria. Immunoblotting analyses were performed with the indicated antibodies. The images were quantified by scanning densitometry.

FIGS. 13A-D. R39/K41 in the Presequence of PGK1 Is Required for Mitochondrial Translocation of PGK1. FIG. 13A. The schematic structure of PGK1 shows positively charged R39, K41, and K48 in the N-terminal a helix of PGK1. FIG. 13B. U251 cells expressing the indicated V5-tagged PGK1 proteins were treated with or without hypoxia for 6 h. Mitochondria fractions and total cell lysates were prepared. Immunoblotting analyses were performed with the indicated antibodies. FIG. 13C. The structure of PGK1 (Protein Data Bank ID: 2zgv) shows that PGK1 R39/K41 residues (yellow spheres) are not exposed on the surface of PGK1 protein. FIG. 13D. Purified GST-PGK1 S203D was mixed with or without purified PIN1, which was followed by immunoprecipitation with the specific anti-PGK1 antibody that recognizes 38-QRIKAA-43. Immunoblotting analysis with an anti-GST antibody was performed.

FIGS. 14A-P. Mitochondrial PGK1 Phosphorylates PDHK1 at T338. FIG. 14A. U87 cells with or without PGK1 shRNA and with or without reconstituted expression of WT rPGK1 or rPGK1 R39/K41A were stimulated with or without hypoxia for 6 h. Mitochondrial fractions of the cells were prepared and activity of PDH complex-mediated conversion of ¹⁴C-labeled pyruvate into ¹⁴C-labeled CO₂ was measured. Data represent the means±SD of three independent experiments. *p<0.01. Immunoblotting analyses were performed with the indicated antibodies. FIG. 14B. U87 cells (left panel) or U87/EGFR (right panel) cells expressing PGK1 shRNA with or without reconstituted expression of WT rPGK1 and the indicated mutants were stimulated with or without hypoxia for 6 h (left panel) or EGF (100 ng/ml) (right panel) for 6 h. These cells were incubated with [1-¹⁴C]-pyruvate for 2 h. [1-¹⁴C]CO₂ production rates were measured. FIG. 14C. U87 cells expressing SFB-PDHK1, SFB-PDHK2, SFB-PDHK3, or SFB-PDHK4 were stimulated with or without hypoxia for 6 h. Mitochondrial fractions of these cells were prepared. SFB pull-down analyses with streptavidin-conjugated beads were performed. Immunoblotting analyses were performed with the indicated antibodies. FIG. 14D. In vitro phosphorylation analyses were performed by mixing bacterially purified His-PGK1 and SUMO-PDHK1 under kinase assay condition in the presence of [γ³²P]ATP. Phospho-amino acid analysis of gel-isolated ³²P-phosphorylated SUMO-PDHK1 was performed. The left hand panel shows the stained phosphor-amino acid markers. On the autoradiogram in the right hand panel, the green circle indicates pSer; the red circle indicates pThr; and the black circle indicates pTyr. FIG. 14E. In vitro phosphorylation analyses with autoradiography were performed by mixing purified WT PGK1 or PGK1 T378P with purified WT PDHK1, PDHK1 S337A, or PDHK1 T338A in the presence of [γ³²P]ATP. FIG. 14F. In vitro phosphorylation analyses were performed by mixing purified PGK1 with purified PDHK1 in the presence or absence of 3-PG (0.5 mM). Immunoblotting analyses were performed with the indicated antibodies. FIG. 14G. In vitro phosphorylation analyses were performed by mixing purified WT PGK1 or PGK1 T378P with purified PDHK1 in the presence of glyceraldehyde 3-phosphate, NAD+, and glyceraldehyde phosphate dehydrogenase (GAPDH). Immunoblotting analyses were performed with the indicated antibodies. FIG. 14H. Mitochondria fraction prepared from U87 cells was treated with or without ATPase (1 unit/mg lysate protein) before incubating with purified WT PGK1. Immunoblotting analyses were performed with the indicated antibodies. FIG. 14I. Km of PGK1 and representative plotting of 1/V vs. 1/[ATP]. FIG. 14J. ATP concentrations in mitoplast isolated from U87 or U251 cells treated with or without hypoxia for 6 h were measured. FIG. 14K. Mitochondria fractions of U87 cells treated with or without hypoxia for 6 h were immunodepleted with or without an anti-PDHK1 pT338 antibody. Immunoblotting analyses were performed with the indicated antibodies. FIG. 14L. U87 and U251 cells expressing PGK1 shRNA were reconstituted to express WT rPGK1 or rPGK1 T378P. Immunoblotting analyses were performed with the indicated antibodies. FIGS. 14M-N. U87 and U251 cells with PGK1 shRNA and with reconstituted expression of WT rPGK1 or rPGK1 T378P were stimulated with or without hypoxia for 6 h. Mitochondria fractions were prepared. Immunoblotting analyses were performed with the indicated antibodies. FIG. 14O. The glycolytic activities of bacterially purified WT PGK1, PGK1 T378P, PGK1 S203A, and PGK1 R39/K41A were measured. Data represent the means±SD of three independent experiments. Immunoblotting analyses were performed with an anti-PGK1 antibody. FIG. 14P. U87 cells were stimulated with or without hypoxia for 6 h. Immunoblotting analyses of mitochondrial lysates were performed with the indicated antibodies.

FIGS. 15A-C. Mitochondrial PGK1 Results in Enhanced PDH Phosphorylation in a PDHK1 T338 phosphorylation-dependent Manner. FIG. 15A. U87 and U251 cells expressing PGK1 shRNA with or without reconstituted expression of WT rPGK1 or rPGK1 T378P were stimulated with or without hypoxia for 6 h. Immunoblotting analyses of mitochondrial lysates were performed with the indicated antibodies. FIG. 15B. U87 expressing PGK1 shRNA with or without reconstituted expression of WT rPGK1 or rPGK1 R39/K41A were stimulated with or without hypoxia for 6 h. Immunoblotting analyses of mitochondrial lysates were performed with the indicated antibodies. FIG. 15C. U87 expressing PGK1 shRNA with reconstituted expression of WT rPGK1 or rPGK1 R39/K41A were stimulated with or without hypoxia for 24 h. Immunoblotting analyses of mitochondrial lysates were performed with the indicated antibodies.

FIGS. 16A-M. PGK1-Mediated PDHK1 Phosphorylation Inhibits Mitochondrial Pyruvate Metabolism and Promotes Glycolysis. FIGS. 16A-K. Data represent the means±SD of three independent experiments. *p<0.01. FIG. 16A. U87 cells (left panel) or U87/EGFR (right panel) cells expressing PDHK1 shRNA with or without reconstituted expression of WT rPDHK1 and the indicated mutants were stimulated with or without hypoxia for 6 h (left panel) or EGF (100 ng/ml) (right panel) for 6 h. These cells were incubated with [1-¹⁴C]-pyruvate for 2 h. [1-¹⁴C]CO₂ production rates were measured. FIG. 16B. U251 cells expressing PDHK1 shRNA with or without reconstituted expression of WT rPDHK1 or rPDHK1 T338A were stimulated with or without hypoxia for 6 h. Levels of mitochondrial acetyl-CoA were measured. FIG. 16C. U251 cells expressing PDHK1 shRNA with or without reconstituted expression of WT rPDHK1 or rPDHK1 T338A were stimulated with or without hypoxia for 24 h. Levels of mitochondrial ROS were measured. FIG. 16D. Endogenous PDHK1-depleted U87 cells were reconstituted with different expression levels of WT rPDHK1 or rPDHK1 T338A (left panel) and were stimulated with hypoxia for 24 h. Mitochondrial ROS production of the cells under hypoxic conditions was measured (right panel). L: low expression; H: high expression. Immunoblotting analyses were performed with the indicated antibodies. FIG. 16E. Endogenous PGK1-depleted U87 cells with reconstituted expression of WT rPGK1 or rPGK1 R39/K41A were stimulated with or without hypoxia for 24 h. Levels of mitochondrial ROS were measured. FIG. 16F. U87 cells with or without PGK1 depletion were stimulated with hypoxia for 6 h. The indicated concentrations of pyruvate were added into the isolated mitochondria. Levels of mitochondrial ROS were measured. FIG. 16G. U87 cells with or without PGK1 depletion were stimulated with hypoxia for 6 h. The indicated concentrations of pyruvate were added into the isolated mitochondria. Levels of mitochondrial membrane potential were measured. ΔΨm, mitochondrial membrane potential. FIG. 16H. Endogenous PGK1-depleted U87/EGFR cells with reconstituted expression of WT rPGK1 or rPGK1 S203A were pretreated with DCA (5 mM) for 1 h before EGF (100 ng/ml) stimulation for 6 h. Mitochondrial OCR was measured. FIG. 16I. U251 cells expressing PGK1 shRNA (left panel) or PDHK1 shRNA (right panel) with or without reconstituted expression of their WT counterparts and the indicated mutants were stimulated with or without hypoxia for 6 h. Levels of cytosolic pyruvate were measured. FIG. 16J. U251 cells expressing PGK1 shRNA (left panel) or PDHK1 shRNA (right panel) with or without reconstituted expression of their WT counterparts and the indicated mutants were cultured in no-serum DMEM during hypoxia for 6 h. The media were collected for analysis of lactate production. FIG. 16K. U251 cells expressing PGK1 shRNA with reconstituted expression of WT rPGK1 or rPGK1 S203D were cultured in no-serum DMEM under normoxic condition for 6 h. The media were collected for analysis of lactate production. FIG. 16L. U87/EGFR cells expressing PGK1 shRNA (left panel) or PDHK1 shRNA (right panel) with or without reconstituted expression of their WT counterparts and the indicated mutants were stimulated with or without EGF (100 ng/ml) for 6 h. Mitochondria fractions were prepared and activity of PDH complex-mediated conversion of ¹⁴C-labeled pyruvate into ¹⁴C-labeled CO₂ was measured. Immunoblotting analyses were performed with the indicated antibodies. FIG. 16M. U87/EGFR expressing PGK1 shRNA (left panel) or PDHK1 shRNA (right panel) with or without reconstituted expression of their WT counterparts and the indicated mutants were cultured in no-serum DMEM during EGF (100 ng/ml) stimulation for 6 h. The media were collected for analysis of lactate production.

FIGS. 17A-H. Mitochondrial PGK1-Dependent PDHK1 Phosphorylation Promotes Cell Proliferation and Brain Tumorigenesis. FIGS. 17A-C. Data represent the means ±SD of three independent experiments. FIG. 17A. A total of 2×10⁵ U251 cells with or without PGK1 shRNA (left panel) or PDHK1 shRNA (right panel) expression and with or without reconstituted expression of their WT counterparts and the indicated mutants were plated for 4 days under hypoxic conditions. The cells were then collected and counted. FIG. 17B. A total of 2×10⁵ endogenous PGK1-depleted U87 cells with reconstituted expression of WT rPGK1 or rPGK1 R39/K41A were plated and treated with or without DTT (1 mM) during hypoxia for 4 days. The cells were then collected and counted. FIG. 17C. Mitochondria fractions and total cell lysates from endogenous PGK1-depleted U87/EGFRvIII cells with reconstituted expression of WT rPGK1 or rPGK1 S203A were prepared. Immunoblotting analyses were performed with the indicated antibodies (left panel). The indicated U87/EGFRvIII cells (2×10⁵) were plated for 3 days and were counted (right panel). FIG. 17D. A total of 2×10⁵ endogenous PGK1-depleted U87 cells with reconstituted expression of WT rPGK1 or rPGK1 S203D were plated and starved with or without glutamine for 3 days. The cells were then collected and counted. #p<0.05. FIG. 17E. GSC11 cells with PGK1 shRNA (upper panel) or PDHK1 shRNA (bottom panel) expression were reconstituted the expression of their WT counterparts and the indicated mutants. Immunoblotting analyses of total cell lysates were performed with the indicated antibodies. FIG. 17F. A total of 1×10⁶ GSC11 cells with or without PGK1 shRNA (upper panel) or PDHK1 shRNA (lower panel) expression and with or without reconstituted expression of their WT counterparts and the indicated mutants were intracranially injected into athymic nude mice for each group. After 21 days, the mice were sacrificed and examined for tumor growth. H&E-stained coronal brain sections show representative tumor xenografts. Tumor volume was calculated using length a and width b: V=ab²/2. FIG. 17G. IHC analyses of tumor tissues were performed with an anti-Ki67 antibody. Ki67-positive cells were quantified in 10 microscope fields. FIG. 17H. TUNEL analyses of the indicated tumor tissues were performed. Apoptotic cells were stained brown. Apoptotic cells were quantified in 10 microscope fields.

FIG. 18. ERK-mediated PGK1 Phosphorylation and PGK1-Dependent PDHK1 Phosphorylation Correlates with PDH Phosphorylation. IHC staining with anti-phospho-PGK1 S203, anti-phospho-PDHK1 T338, and anti-phospho-PDH S293 antibodies was performed on 50 human primary GBM specimens. Semi-quantitative scoring (scale, 1-8) was performed (Pearson product moment correlation test; upper left panel, r=0.66, p<0.01; upper right panel, r=0.71, p<0.01; lower panel, r=0.73, p<0.01). Note that some of the dots on the graphs represent more than one specimen (i.e., some scores overlapped).

FIGS. 19A-C. PGK1 Phosphorylates Histone H3, CDC45, and Beclin-1. FIG. 19A. Western blot for histone H3 pS10 showing that PGK1 phosphorylates histone H3 at S10. Under control conditions, EGF stimulated phosphorylation of histone H3 at S10, however this effect was greatly diminished by knock down of PGK1 using shRNA. FIG. 19B. Western blots and autoradiography blot for CDC45 showing that PGK1 phosphorylates CDC45 at PS386. Purified wild-type PGK1, but not PGK1 kinase-dead (KD) mutant, phosphorylated purified wild-type CDC45, but not CDC45 S386A, in the presence of [γ³²P]-ATP. FIG. 19C. Western blots and autoradiography blot for Beclin-1 showing that PGK1 phosphorylates Beclin-1 at S30. Purified PGK1 phosphorylated purified wild-type Beclin-1 in the presence of [γ³²P]-ATP. Beclin-1 S30A mutant was largely resistant to phosphorylation by PGK1.

FIGS. 20A-C. FIGS. 20A-B. In vitro protein kinase assays were performed by mixing purified recombinant PGK1 WT, Y324F or T378P with [γ³²P]-ATP. PGK1 Y324 phosphorylation was detected by autoradiography (FIG. 20A) or a PGK1 pY324 antibody (FIG. 20B). FIG. 20C. PGK1 enzyme activity assay. Purified recombinant PGK1 WT, Y324F or T378P were incubated in 100 μl of reaction buffer (50 mM Tris-HCl [pH 7.5], 5 mM MgCl₂, 5 mM ATP, 0.2 mM NADH, 10 mM glycerol-3-phosphate, and 10 U of GAPDH) at 25° C. in a 96-well plate and read at 339 nm in kinetic mode for 5 min.

### DESCRIPTION OF ILLUSTRATIVE EMBODIMENTS

The Warburg effect is characterized by increased glucose uptake and lactate production combined with suppressed mitochondrial pyruvate metabolism. However, the mechanism by which cytosolic glycolysis is coordinately regulated with mitochondrial metabolism remains elusive. Hypoxia, EGFR activation, and expression of K-Ras G12V and B-Raf V600E induce mitochondrial translocation of phosphoglycerate kinase 1 (PGK1); this is mediated by ERK-dependent phosphorylation of PGK1 at S203 and subsequent PIN1-mediated cis-trans isomerization of pS203.P204, allowing interaction of R39/K41 in the PGK1 N-terminal a-helix with the TOM complex. In the mitochondria, PGK1 acts as a protein kinase to phosphorylate pyruvate dehydrogenase kinase 1 (PDHK1) at T338, which activates PDHK1 to phosphorylate and inhibit the pyruvate dehydrogenase (PDH) complex. This PGK1-mediated PDH inhibition reduces the utilization of pyruvate in mitochondria, suppresses reactive oxygen species production, and increases glycolysis and glutaminolysis-driven lipid synthesis, leading to enhanced tumorigenesis. Furthermore, PGK1 S203 and PDHK1 T338 phosphorylation levels correlate with PDH S293 phosphorylation levels and poor prognosis in glioblastoma patients. These findings unearth PGK1 functioning as a protein kinase in coordinating glycolysis and the TCA cycle, which is instrumental in cancer metabolism and tumorigenesis.

This invention demonstrates that PGK1 is a metabolic enzyme containing protein kinase activity that can be targeted for cancer treatment. PGK1 is a protein kinase for tumorigenesis; phosphorylation events PGK1 pS203, PDHK pT338, CDC45 pS386, Beclin1 pS30, and Histone H3 pS10 are biomarkers for prognosis and personalized therapy.

### I. Aspects of the Invention

A tumor cell mass, developing initially in a vascular environment, can become severely hypoxic as a result of massive expansion distant from the vasculature (Brahimi-Hom et al., 2007). To survive this hypoxic stress and to support tumor cell growth, the tumor cells upregulate glycolysis and suppress pyruvate metabolism and oxidative phosphorylation in the mitochondria so that pyruvate is converted to lactate in the cytoplasm (Gatenby and Gillies, 2004). In normoxic condition, activation of receptor tyrosine kinases or the presence of prevalent K-Ras and B-Raf mutations promotes the Warburg effect (Yang et al., 2012; Yaffe et al., 1997; Tani et al., 1985). Demonstrated herein, and without being bound by theory, is a previously unknown mechanism underlying the coordinated regulation of glycolysis and the TCA cycle by subcellular compartment-dependent regulation of the glycolytic enzyme PGK1: hypoxic stress, activation of EGFR, or expression of K-Ras G12V or B-Raf V600E results in ERK1/2-dependent phosphorylation of PGK1 at S203, leading to PIN1-dependent PGK1 cis-trans isomerization, binding of PGK1 to the TOM complex, and subsequently mitochondrial translocation of PGK1. In mitochondria, PGK1 directly interacts with and phosphorylates PDHK1 at T338. This phosphorylation leads to enhanced PDHK1 activity and PDHK1-mediated PDH phosphorylation, which results in the suppression of PDH-dependent pyruvate utilization and ROS production in mitochondria and the increase of glycolysis and EGF-induced and glutaminolysis-promoted lipid synthesis. This metabolic alteration promotes cell proliferation and tumorigenesis (FIG. 8). Along with the previous findings that PKM2 functions as a histone kinase (Yang et al., 2012), the demonstration that PGK1 functions as a protein kinase highlights the dual roles of PGK1 and PKM2, which act as both glycolytic enzymes and protein kinases in cell metabolism and proliferation, expands the kinome with an important family branch, and greatly impacts the understanding of protein enzymes with “multiple faces” in controlling cellular functions.

Hypoxic cells increase glycolysis with suppressed cellular respiration. The increased glycolysis had been attributed primarily to the HIF1α- or HIF2α-dependent glycolytic enzyme expression, whereas the suppressed cellular respiration was thought to result from the paucity of oxygen required for accepting electrons from the mitochondrial respiratory chain and from the inhibition of mitochondrial pyruvate metabolism and respiration, which can be regulated by several mechanisms, including HIF1α-upregulated PDHK1 expression (Kim et al., 2006; Semenza, 2008). Deficiency of mitochondrial translocation of PGK1 induced by expressing PGK1 R39/K41A, which had no effect on hypoxia-enhanced PDHK1 expression, largely reduced hypoxia-induced PDH phosphorylation. These results indicated that overexpression of PDHK1 by itself is not sufficient to maximize its cellular activity in mitochondria and that PGK1-mediated phosphorylation of PDHK1 and hypoxia-enhanced PDHK1 expression have synergistic effects on regulating PDHK1 activity.

Although tumor cells can regulate glycolysis and mitochondria simultaneously via HIF-regulated expression of glycolytic genes and mitochondrial enzymes under hypoxic conditions, this regulation, which does not occur in normoxic conditions, is a chronic response and requires regulation of gene transcription. Activation of EGFR, K-Ras, and B-Raf under normoxic conditions or hypoxia stimulation induced an immediate or acute response of tumor cells by rapid mitochondrial translocation of PGK1, which led to inhibition of mitochondrial pyruvate metabolism, shuttling of mitochondrial pyruvate to cytosol for lactate production, and increase of glutaminolysis-promoted fatty acid synthesis. Thus, these findings provide a new concept of integrated regulation of glycolysis, the TCA cycle, and glutaminolysis and provide a critical and novel insight into the Warburg effect induced by prevalent oncogenes, such as EGFR, K-Ras, and B-Raf. Given that PGK1 expression is upregulated in human cancer and is associated with tumor metastasis and drug resistance (Zhang et al., 2005; Hwang et al., 2006; Duan et al., 2002; Zieker et al., 2010; Ahmad et al., 2013; Ai et al., 2011), these findings-demonstrating that PGK1-dependent PDHK1 T338 phosphorylation promotes tumor cell proliferation and tumorigenesis and that the phosphorylation levels of PGK1 S203 and PDHK1 T338 correlate with glioblastoma prognosis-provide a molecular basis for improved diagnosis and treatment of human cancer.

### II. Detection Methods

In certain embodiments, the method comprises the steps of obtaining a biological sample from a mammal to be tested; detecting the level of phosphorylation of a PGK1, PDHK1, PDH, CDC45, Histone H3, or Beclin-1 protein in the sample. In one embodiment, the biological sample is a cell sample from a tumor in the mammal. As used herein the phrase “selectively measuring” refers to methods wherein only a finite number of protein phosphorylation events are measured rather than assaying essentially all protein phosphorylation in a sample. For example, in some aspects “selectively measuring” protein phosphorylation events can refer to measuring no more than 100, 75, 50, 25, 15, 10, 5, or 2 different protein phosphorylation events.

In another embodiment of the methods described herein, detecting the presence a phosphorylated protein in a biological sample obtained from an individual comprises determining the level of a phosphorylated polypeptide in the sample. The level of a phosphorylated protein can be determined by contacting the sample with an antibody that specifically binds to the phosphorylated polypeptide and determining the amount of bound antibody, e.g., by detecting or measuring the formation of the complex between the antibody and the polypeptide. The antibodies can be labeled (e.g., radioactive, fluorescently, biotinylated or HRP-conjugated) to facilitate detection of the complex. Appropriate assay systems for detecting polypeptide levels include, but are not limited to, flow cytometry, Enzyme-Linked Immunosorbent Assay (ELISA), competition ELISA assays, Radioimmuno-Assays (RIA), immunofluorescence, gel electrophoresis, Western blot, and chemiluminescent assays, bioluminescent assays, immunohistochemical assays that involve assaying a phosphorylated protein in a sample using antibodies having specificity for the polypeptide product. Numerous methods and devices are well known to the skilled artisan for the detection and analysis of the instant invention. With regard to polypeptides or proteins in test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as but not limited to, biosensors and optical immunoassays, may be employed to determine the presence or amount of analytes without the need for a labeled molecule.

Alternatively, the level of phosphorylation of a PGK1, PDHK1, PDH, CDC45, Histone H3, or Beclin-1 polypeptide may be detected using mass spectrometric analysis. Mass spectrometric analysis has been used for the detection of proteins in serum samples. Mass spectroscopy methods include Surface Enhanced Laser Desorption Ionization (SELDI) mass spectrometry (MS), SELDI time-of-flight mass spectrometry (TOF-MS), Maldi Qq TOF, MS/MS, TOF-TOF, ESI-Q-TOF and ION-TRAP.

A polypeptide can be detected and quantified by any of a number of means known to those of skill in the art, including analytic biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, flow cytometry, FACS, western blotting, and the like.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye. The labeled biomarker proteins are incubated with the antibody microarray. After washes to remove the unbound proteins, the microarray is scanned. The raw fluorescent intensity data may be converted into expression values using means known in the art.

### III. Methods of Treating

Certain aspects of the present invention can be used to identify and/or treat a disease or disorder based on the phosphorylation state of S203 of PGK1, Y324 of PGK1, T338 of PDHK1, S293 of PDH, S386 of CDC45, S10 of Histone H3, and/or S30 of Beclin-1. Other aspects of the present invention provide for treating a cancer patient with PGK1, MEK/ERK, EGFR, and/or PIN1 inhibitors.

The term “subject” or “patient” as used herein refers to any individual to which the subject methods are performed. Generally the patient is human, although as will be appreciated by those in the art, the patient may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect. A tissue, tumor, or cell can be contacted with one or more compositions or pharmacological formulation(s) comprising one or more of the agents, or by contacting the tissue, tumor, and/or cell with two or more distinct compositions or formulations. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

The methods described herein are useful in treating cancer. Generally, the terms “cancer” and “cancerous” refer to or describe the physiological condition in mammals that is typically characterized by unregulated cell growth. More specifically, cancers that are treated using any one or more PGK1, MEK/ERK, EGFR, and/or PIN1 inhibitors, or variants thereof, and in connection with the methods provided herein include, but are not limited to, solid tumors, metastatic cancers, or non-metastatic cancers. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; lymphoma; blastoma; sarcoma; carcinoma, undifferentiated; meningioma; brain cancer; oropharyngeal cancer; nasopharyngeal cancer; renal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; non-small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and hairy cell leukemia.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Regarding neoplastic condition treatment, depending on the stage of the neoplastic condition, neoplastic condition treatment involves one or a combination of the following therapies: surgery to remove the neoplastic tissue, radiation therapy, and chemotherapy. Other therapeutic regimens may be combined with the administration of the anticancer agents, e.g., therapeutic compositions and chemotherapeutic agents. For example, the patient to be treated with such anti-cancer agents may also receive radiation therapy and/or may undergo surgery.

For the prevention or treatment of disease, the appropriate dosage of a therapeutic composition, e.g., a PGK1, MEK/ERK, EGFR, and/or PIN1 inhibitor, will depend on the type of disease to be treated, as defined above, the severity and course of the disease, whether the agent is administered for preventive or therapeutic purposes, previous therapy, the patient's clinical history and response to the agent, and the discretion of the physician. The agent is suitably administered to the patient at one time or over a series of treatments.

### IV. Therapeutics of the Embodiments

Certain aspects of the embodiments concern administering a targeted therapy to a patient determined to comprise one or more biomarkers of the embodiments. In some aspects, a patient identified to have a cancer expressing activated PGK2 (or a biomarker thereof) is administered one or more of a PGK1 inhibitor, a MEK/ERK inhibitor, a EGFR inhibitor, or a PIN1 inhibitor therapy. Some specific targeted therapies for use according to the embodiments are provided below.

A. MEK/ERK Kinase Inhibitors

MEK inhibitors, which include inhibitors of mitogen-activated protein kinase kinase (MAPK/ERK kinase or MEK) or its related signaling pathways like MAPK cascade, may be used in certain aspects of the embodiments. Mitogen-activated protein kinase kinase (sic) is a kinase enzyme which phosphorylates mitogen-activated protein kinase. It is also known as MAP2K. Extracellular stimuli lead to activation of a MAP kinase via a signaling cascade (“MAPK cascade”) composed of MAP kinase, MAP kinase kinase (MEK, MKK, MEKK, or MAP2K), and MAP kinase kinase kinase (MKKK or MAP3K).

A MEK inhibitor herein refers to MEK inhibitors in general. Thus, a MEK inhibitor refers to any inhibitor of a member of the MEK family of protein kinases, including MEK1, MEK2 and MEK5. Reference is also made to MEK1, MEK2 and MEK5 inhibitors. Examples of suitable MEK inhibitors, already known in the art, include the MEK1 inhibitors PD184352 and PD98059, inhibitors of MEK1 and MEK2 U0126 and SL327, and those discussed in Davies et al. (2000).

In particular, PD184352 and PD0325901 have been found to have a high degree of specificity and potency when compared to other known MEK inhibitors (Bain et al., 2007). Other MEK inhibitors and classes of MEK inhibitors are described in Zhang et al. (2000).

Inhibitors of MEK can include antibodies to, dominant negative variants of, and siRNA and antisense nucleic acids that suppress expression of MEK. Specific examples of MEK inhibitors include, but are not limited to, PD0325901 (see, e.g., Rinehart et al., 2004), PD98059 (available, e.g., from Cell Signaling Technology), U0126 (available, for example, from Cell Signaling Technology), SL327 (available, e.g., from Sigma-Aldrich), ARRY-162 (available, e.g., from Array Biopharma), PD184161 (see, e.g., Klein et al., 2006), PD184352 (CI-1040) (see, e.g., Mattingly et al., 2006), sunitinib (AZD6244/ARRY-142886/ARRY-886; see, e.g., Voss, et al., US2008/004287 incorporated herein by reference), sorafenib (see, Voss supra), Vandetanib (see, Voss supra), pazopanib (see, e.g., Voss supra), Axitinib (see, Voss supra), PTK787 (see, Voss supra), refametinib (BAY-86-9766/RDEA-119), Pimasertib (also known as AS703026 or MSC1936369B), and trametinib (GSK-1120212).

Currently, several MEK inhibitors are undergoing clinical trial evaluations. CI-1040 has been evaluated in Phase I and II clinical trials for cancer (see, e.g., Rinehart et al., 2004). Other MEK/ERK inhibitors being evaluated (e.g., in clinical trials) include PD 184352 (see, e.g., English and Cobb, 2002), BAY 43-9006 (see, e.g., Chow et al., 2001), PD-325901 (also PD0325901), ARRY-438162, RDEA119, RDEA-436, RO5126766, XL518, AZD8330 (also ARRY-704), GDC-0973, RDEA119, PD18416, SCH 900353, RG-7167, WX-554, E-6201, AS-703988, BI-847325, TAK-733, RG-7304, or FR180204.

B. PIN1 Inhibitors

Further aspects of the embodiments concern Pin1 inhibitors and the administration of such inhibitors. Examples of inhibitors of Pin1 include, without limitation, TME-001 (2-(3-chloro-4-fluoro-phenyl)-isothiazol-3-one; see, Mori et al., 2011), 5′-nitro-indirubinoxime (Yoon et al., 2012) and cyclohexyl ketone substrate analogue inhibitors, such as Ac-pSer-[C═OCH]-Pip-tryptamine (Xu et al., 2012). Xu et al. (2011) also describe a Pin1 inhibitor having the structure R-pSer-WI[CH₂N]-Pro-2-(indol-3-yl)ethylamine, wherein R is fluorenylmethoxycarbonyl (Fmoc) or Ac. Peptides such as, disulfide-cyclized peptides, have also been demonstrated as an effective Pin1 inhibitors and may be used in accordance with the present embodiments (see, e.g., Duncan et al. (2011), incorporated herein by reference).

C. Additional Targeted Inhibitors

Targeted inhibition can likewise be achieved using targeted inhibitory RNA therapies (e.g., through the administration or expression of micro RNAs (miRNAs) or small interfering RNAs (siRNAs) to a particular gene or pathway). Inhibition of, for example, PGK1, MEK/ERK, EGFR, or PIN1 can be conveniently achieved using RNA-mediated interference. Typically, a double-stranded RNA molecule complementary to all or part of a target mRNA is introduced into cancer cells, thus promoting specific degradation of mRNA molecules. This post-transcriptional mechanism results in reduced or abolished expression of the targeted mRNA and the corresponding encoded protein.

Moreover a number of assays for identifying new targeted inhibitor, including, e.g., PGK1, MEK/ERK, EGFR, or PIN1 inhibitors, are known. For example, Davies et al. (2000) describes kinase assays in which a kinase is incubated in the presence of a peptide substrate and radiolabeled ATP. Phosphorylation of the substrate by the kinase results in incorporation of the label into the substrate. Aliquots of each reaction are immobilized on phosphocellulose paper and washed in phosphoric acid to remove free ATP. The activity of the substrate following incubation is then measured and provides an indication of kinase activity. The relative kinase activity in the presence and absence of candidate kinase inhibitors can be readily determined using such an assay. Downey et al. (1996) also describes assays for kinase activity which can be used to identify kinase inhibitors that may be used in accordance with the embodiments.

D. Prodrugs

Compounds, such as targeted inhibitors of the present embodiments may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. In general, such prodrugs will be functional derivatives of the metabolic pathway inhibitors of the embodiments, which are readily convertible in vivo into the active inhibitor. Conventional procedures for the selection and preparation of suitable prodrug derivatives are described, for example, in “Design of Prodrugs”, ed. H. Bundgaard, Elsevier, 1985; Huttunen et al., 2011; and Hsieh et al., 2009, each of which is incorporated herein by reference in its entirety.

A prodrug may be a pharmacologically inactive derivative of a biologically active inhibitor (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality. Thus, prodrugs of the compounds employed in the embodiments may be prepared by modifying functional groups present in the compound in such a way that the modifications are cleaved, either in routine manipulation or in vivo, to the parent compound. Prodrugs include, for example, compounds described herein in which a hydroxy, amino, or carboxy group is bonded to any group that, when the prodrug is administered to a subject, cleaves to form a hydroxy, amino, or carboxylic acid, respectively. Thus, the invention contemplates prodrugs of compounds of the present invention as well as methods of delivering prodrugs.

E. Inhibitory Oligonucleotides

An inhibitory oligonucleotide can inhibit the transcription or translation of a gene in a cell. An oligonucleotide may be from 5 to 50 or more nucleotides long, and in certain embodiments from 7 to 30 nucleotides long. In certain embodiments, the oligonucleotide maybe 7, 8, 9, 10, 11, 12, 13, 14, 15, 16, 17, 18, 19, 20, 21, 22, 23, 24, 25, 26, 27, 28, 29, or 30 nucleotides long. The oligonucleotide may comprise a nucleic acid and/or a nucleic acid analog. Typically, an inhibitory oligonucleotide will inhibit the translation of a single gene (e.g., PGK1) within a cell; however, in certain embodiments, an inhibitory oligonucleotide may inhibit the translation of more than one gene within a cell.

Within an oligonucleotide, the components of the oligonucleotide need not be of the same type or homogenous throughout (e.g., an oligonucleotide may comprise a nucleotide and a nucleic acid or nucleotide analog). In certain embodiments of the present invention, the oligonucleotide may comprise only a single nucleic acid or nucleic acid analog. The inhibitory oligonucleotide may comprise 5, 6, 7, 8, 9, 10, 11, 12, 13, 14, 15, 16, 17, 18, 19, 20, 25, 30 or more contiguous nucleobases, including all ranges therebetween, that hybridize with a complementary nucleic acid to form a double-stranded structure.

### III. Combination Treatments

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. A tissue, tumor, or cell can be contacted with one or more compositions or pharmacological formulation(s) comprising one or more of the agents or by contacting the tissue, tumor, and/or cell with two or more distinct compositions or formulations. Also, it is contemplated that such a combination therapy can be used in conjunction with radiotherapy, surgical therapy, or immunotherapy.

Administration in combination can include simultaneous administration of two or more agents in the same dosage form, simultaneous administration in separate dosage forms, and separate administration. That is, the subject therapeutic composition and another therapeutic agent can be formulated together in the same dosage form and administered simultaneously. Alternatively, subject therapeutic composition and another therapeutic agent can be simultaneously administered, wherein both the agents are present in separate formulations. In another alternative, the therapeutic agent can be administered just followed by the other therapeutic agent or vice versa. In the separate administration protocol, the subject therapeutic composition and another therapeutic agent may be administered a few minutes apart, or a few hours apart, or a few days apart.

An anti-cancer first treatment may be administered before, during, after, or in various combinations relative to a second anti-cancer treatment. The administrations may be in intervals ranging from concurrently to minutes to days to weeks. In embodiments where the first treatment is provided to a patient separately from the second treatment, one would generally ensure that a significant period of time did not expire between the time of each delivery, such that the two compounds would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a PGK1, MEK/ERK, EGFR, and/or PIN1 inhibitor is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies may be used in combination or in conjunction with methods of the invention. In the context of cancer treatment, immunotherapeutics, generally, rely on the use of immune effector cells and molecules to target and destroy cancer cells. Rituximab (Rituxan®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hanibuchi et al., 1998; U.S. Pat. No. 5,824,311). It is contemplated that one or more anti-cancer therapies may be employed with the antibody therapies described herein.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present invention, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present invention to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present invention to improve the treatment efficacy.

### IV. Kits and Diagnostics

In various aspects of the invention, a kit is envisioned containing diagnostic agents, therapeutic agents, and/or other therapeutic and delivery agents. In some embodiments, the present invention contemplates a kit for preparing and/or administering a therapy of the invention. The kit may comprise reagents capable of use in administering an active or effective agent(s) of the invention. Reagents of the kit may include at least one inhibitor of gene expression, one or more anti-cancer component of a combination therapy, as well as reagents to prepare, formulate, and/or administer the components of the invention or perform one or more steps of the inventive methods.

In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods, and will follow substantially the same procedures as described herein or are known to those of ordinary skill.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Materials.

Rabbit polyclonal antibodies recognizing PGK1, PGK1 38-QRIKAA-43, phospho-PGK1 S203, PDHK1, phospho-PDHK1 T338, phospho-PDHK1 Y243, EGFR, phospho-EGFR Y1172, and HA were obtained from Signalway Antibody (College Park, Md.). Rotenone, rabbit polyclonal antibodies recognizing PGK1, PDHK1, COX IV, TIMM22, V5, PDH Ela, PDH Ela pS293, and mouse monoclonal antibodies recognizing rabbit IgG with native conformation were obtained from Abcam (Cambridge, Mass.). Rabbit polyclonal antibodies recognizing MAPK/APK2, MAPK/APK2 pT222, c-Jun, and c-Jun pS73 were purchased from Cell Signaling Technology (Danvers, Mass.). Mouse antibodies recognizing HIF1α, cytochrome c, and phospho-serine were obtained from BD Biosciences (Bedford, Mass.). Monoclonal antibodies against GST, tubulin, ERK1/2, pERK1/2, PIN1, and phospho-threonine were purchased from Santa Cruz Biotechnology (Santa Cruz, Calif.). Active Glyceraldehyde 3-Phosphate Dehydrogenase (GAPDH) recombinant protein, mouse monoclonal antibodies for Flag, Triton X-100, a-chymotrypsin, glyceraldehyde 3-phosphate, lithium chloride, pyruvate, malate, EGTA, ATPase, EGF, dichloroacetate (DCA), phosphorenolpyruvate, pyruvate kinase, lactic dehydrogenase, ADP, NADH, and NAD⁺ were purchased from Sigma (St. Louis, Mo.). Rabbit polyclonal antibodies recognizing MnSOD and TOM20, U0126, SP600125, SB203580, AG1478, puromycin, blasticidin, and DNase-free RNase A were purchased from EMD Biosciences (San Diego, Calif.). Proteinase K and MitoTracker Red CMXRos were purchased from Invitrogen (Carlsbad, Calif.). Monoclonal antibodies against TOM20 and Ki67 were purchased from Millipore (San Diego, Calif.). HyFect transfection reagents were from Denville Scientific (Metuchen, N.J.). Purified His-PDH Ela recombinant protein was from SignalChem (Richmond, Canada). Immunogold-labeled anti-rabbit secondary antibodies were purchased from Ted Pella (Redding, Calif.). Synthesized phosphorylated (HHHHHHLEpSPER-pNA; SEQ ID NO: 1) and nonphosphorylated (HHHHHHLESPER-pNA [SEQ ID NO: 1] and HHHHHHLEDPER-pNA [SEQ ID NO: 2]) oligopeptide of PGK1 containing the 5203/P204 motif were purchased from Selleck Chemicals (Houston, Tex.). [γ³²P]ATP was purchased from MP Biochemicals (Santa Ana, Calif.). [1-¹⁴C]-pyruvate was purchased from American Radiolabeled Chemicals (St. Louis, Mo.). DAPI, Alexa Fluor 488 goat anti-rabbit antibody, hyamine hydroxide, scintillation vials, and siRNA targeting HIF1α were purchased from Thermo Fisher Scientific (Pittsburgh, Pa.). Streptavidin beads were purchased from Pierce (Rockford, Ill.).

Cells and Cell Culture Conditions.

U87, U87/EGFR, and U251 human GBM cells, BxPC-3 human pancreatic cancer cells, CHL1 human melanoma cells, and mouse embryo fibroblast (MEF) cells were maintained in Dulbecco's modified Eagle's medium (DMEM) supplemented with 10% dialyzed bovine calf serum without pyruvate (HyClone, Logan, Utah). Human primary GSC11 GBM cells were maintained in DMEM/F-12 50/50 supplemented with B27 (Invitrogen, Carlsbad, Calif.), epidermal growth factor (10 ng/ml), and basic fibroblast growth factor (10 ng/ml). Cells were cultured under normoxic (20% oxygen) or hypoxic (1% oxygen) conditions. For EGF treatments, cell cultures were made quiescent by growing them to confluence, and the medium was replaced with fresh medium containing 0.5% serum for 1 day. EGF at a final concentration of 100 ng/ml was used for cell stimulation.

Transfection.

Cells were plated at a density of 4×10⁵ per 60-mm dish 18 h before transfection. Transfection was performed as previously described (Xia et al., 2007).

Subcellular Fractionation.

Mitochondrial and cytosolic fractions were isolated using a mitochondria/cytosol fractionation kit from BioVision (Mountain View, Calif.). Mitochondrial proteins and cytosolic proteins were used in immunoblotting analyses. For the proteinase K protection assay, the mitochondria pellet was resuspended in hypotonic buffer (10 mM HEPES-KOH [pH 7.4] and 1 mM EDTA) and digested on ice with proteinase K (200 mg/ml) with or without 1% Triton X-100 or digitonin (100 μM) for 20 min. The digest was then precipitated with trichloroacetic acid and analyzed by sodium dodecyl sulfate-polyacrylamide gel electrophoresis (SDS-PAGE) and immunoblotting. Mitochondrial subfractionation was performed as described previously (She et al., 2011). Briefly, isolated mitochondria were resuspended in 10 μM KH₂PO₄ (pH 7.4) for 20 min on ice. An equal volume of iso-osmotic solution (32% sucrose, 30% glycerol, 10 mM MgCl₂) was added and spun at 10,000 g and 4° C. for 10 min. The supernatant was centrifuged at 15,000 g and 4° C. for 1 h; the pellet and supernatant contained outer membrane and intermembrane space proteins, respectively. Then, the pellet from the first-time centrifuge was resuspended in 10 μM KH₂PO₄ (pH 7.4) for 20 min on ice, and iso-osmotic solution was added, followed by centrifuge at 15,000 g and 4° C. for 1 h; the pellet and supernatant contained inner membrane and matrix proteins, respectively.

Mitochondrial [1-¹⁴C]-Pyruvate Conversion Assay.

The [1-¹⁴C]-pyruvate conversion assay was performed as previously described (Pezzato et al., 2009) with minor modification. Briefly, ¹⁴CO₂ production through the PDH complex was measured using isolated mitochondria (1 mg) in the 1 ml mitochondria resuspension buffer (200 mM sucrose, 10 mM Hepes [pH 7.4], 5 mM malate, 2 mM monosodium phosphate, 2 mM ADP, 1 mM EGTA) containing [1-¹⁴C]-pyruvate (0.1 μCi/ml). The incubation mixture in a 2-ml Eppendorf tube was placed at the bottom of a 20-ml scintillation vial with a foil-lined screw cap and maintained in agitation. The ¹⁴CO₂ produced during incubation was trapped by 1 ml of hyamine hydroxide at the bottom of the scintillation vial. The reaction mixture was removed from the scintillation vial and blocked with 0.5 ml of 50% trichloroacetic acid for 1 h. Then, 19 ml of scintillator liquid was added to each scintillation vial and radioactivity was measured on a scintillation counter. The results were normalized based on mitochondrial protein levels measured by Bradford assay using bovine serum albumin as the standard.

Immunogold Transmission Electron Microscopy.

Fixed samples were washed in 0.1 M cacodylate buffer and treated with 0.1% Millipore-filtered buffered tannic acid, postfixed with 1% buffered osmium tetroxide for 30 min, and stained en bloc with 1% Millipore-filtered uranyl acetate. The samples were washed several times in water, dehydrated in increasing concentrations of ethanol, infiltrated, and embedded in LX-112 medium (Ladd Research, Williston, Vt.). The samples were polymerized in a 60° C. oven for 2 days. Ultrathin sections were cut with a Leica Ultracut microtome (Deerfield, Ill.), stained with uranyl acetate and lead citrate in a Leica EM stainer, and examined with a JEM 1010 transmission electron microscope (JEOL, USA, Inc., Peabody, Mass.) at an accelerating voltage of 80 kV. Digital images were obtained using an Advanced Microscopy Techniques imaging system (Danvers, Mass.).

In Vitro Isomerization Assay.

The isomerization rate was shown with the cis-peptide content, which was determined by isomer-specific proteolysis. Cis-peptides were prepared by incubating the peptides with a-chymotrypsin at 0° C. for 2 min to completely hydrolyze the trans isomer at the 4-nitroanilide bond to obtain the pure cis-peptides. The pure cis-peptides were left to re-equilibrate. As the isomerization proceeded, aliquots were taken at the indicated times. Chymostatin was added to inactivate chymotrypsin. The absorbance of the released 4-nitroaniline was measured at 390 nm.

Measurement of ROS.

Harvested cells were washed in TD buffer (25 mM Tris [pH 7.4], 5 mM KCl, 400 μM Na₂HPO₄, and 150 mM NaCl) and then resuspended in TD buffer containing 5 mM MitoSOX (red mitochondrial superoxide indicator) (Invitrogen, M36008) at 37° C. for 15 min. After an additional washing with TD buffer, the cells were plated on 96-well plates and measured with a Synergy HT microplate reader at 485/590 nm (BioTek, Winooski, Vt.). Isolated mitochondria in resuspensions in buffer (200 mM sucrose, 10 mM HEPES [pH 7.4], 5 mM malate, 2 mM monosodium phosphate, 2 mM ADP, 1 mM EGTA) containing different concentrations of pyruvate were incubated at 20° C. for 30 min. Measurement of mitochondrial ROS was performed as the description for harvested cells.

Measurement of Mitochondrial Membrane Potential.

Isolated mitochondria (1 mg) in resuspension buffer (200 mM sucrose, 10 mM HEPES [pH 7.4], 5 mM malate, 2 mM monosodium phosphate, 2 mM ADP, 1 mM EGTA) containing different concentrations of pyruvate were incubated at 20° C. for 30 min. Mitochondrial membrane potential was measured using a mitochondrial membrane potential assay kit (Abcam, Cambridge, Mass.).

Measurement of Lactate Production.

Cells were seeded in culture dishes, and the medium was changed after 6 h with non-serum DMEM. Cells were incubated for 6 h, and the culture medium was then collected for measurement of lactate concentrations. Lactate levels were determined using a BioVision lactate assay kit (BioVision, Milpitas, Calif.).

Measurement of Cytosolic Pyruvate and Mitochondrial Acetyl-CoA Concentrations.

Cells were seeded in culture dishes and cultured during normoxia or hypoxia for 6 h. Cytosolic pyruvate and mitochondrial acetyl-CoA concentrations were measured by using a pyruvate colorimetric assay kit (BioVision) and an acetyl-CoA fluorometric assay kit (BioVision), respectively.

Measurement of PGK1 Activity.

Purified recombinant WT or mutant PGK1 (1 ng) was incubated in 100 μl of reaction buffer (5 mM KH₂PO₄, pH 7.0, 1 mM GAP, 0.3 mM beta-NAD, 0.2 mM ADP, 5 mM MgSO₄, 100 mM glycine, 5 ng/μl GAPDH) at 25° C. in 96-well plate and read at 339 nm in kinetic mode for 5 min.

Mass Spectrometry Analysis.

An in vitro PGK1-phosphorylated sample of purified PDHK1 was digested in-gel in 50 mM ammonium bicarbonate buffer containing Rapigest (Waters Corp., Milford, Mass.) overnight at 37° C. with 200 ng of sequencing-grade modified trypsin (Promega, Madison, Wis.). The digest was analyzed by LC-MS/MS on an Obitrap-Elite mass spectrometer (Thermo Fisher Scientific, Waltham, Mass.). Proteins were identified by database searching of the fragment spectra against the SwissProt protein database (EBI) using Mascot Server v.2.3 (Matrix Science, London, UK) and SEQUEST v.1.27 (University of Washington, Seattle, Wash.) via Proteome Discoverer software v.1.4 (Thermo Fisher Scientific). Phosphopeptide matches were analyzed by using the PhosphoRS algorithm implemented in Proteome Discoverer and manually curated (Taus et al., 2011).

Immunoprecipitation and Immunoblotting Analysis.

Extraction of proteins with a modified buffer from cultured cells was followed by immunoprecipitation and immunoblotting with corresponding antibodies as described previously (Lu et al., 1998).

Streptavidin and GST Pull-Down Assays.

Streptavidin or glutathione agarose beads were incubated with cell lysate (1 mg/ml) or purified protein for 12 h. The beads were washed with the lysate buffer three times.

Cell Proliferation Assay.

A total of 2×10 cells were plated and counted at 4 days after seeding in DMEM with 10% dialyzed bovine calf serum during hypoxia. Data represent the means±SD of three independent experiments.

DNA Constructs and Mutagenesis.

Polymerase chain reaction (PCR)-amplified human PGK1, TOM20, PDHK1, PDHK2, PDHK3, PDHK4, K-Ras G12V, or B-Raf V600E were cloned into pCold I, pGEX-4T-1, pE-SUMO, pcDNA6/His V5 or pcDNA6/SFB vector. pcDNA6/His V5 PDHK1 T338A, pE-SUMO PDHK1 T338A, pGEX-4T-1 PGK1 S203A, pGEX-4T-1 PGK1 S203D, pGEX-4T-1 PGK1 R39/K41A, pGEX-4T-1 PGK1 S203D R39/K41A, pCold I PGK1 S203A, pCold I PGK1 S203D, pCold I PGK1 T378P, pCold I PGK1 R39/K41A, pcDNA6/His V5 PGK1 S203A, pcDNA6/His V5 PGK1 S203D, pcDNA6/His V5 PGK1 T378P, pcDNA6/His V5 PGK1 R39/K41A, pcDNA6/His V5 PGK1 K48A, pcDNA6/SFB PGK1 R39/K41A, pcDNA6/SFB PGK1 K48A, pcDNA6/SFB PGK1 V81/83A, pcDNA6/SFB PGK1 V177/179A, and pcDNA6/SFB PGK1 V278A/I280R/L282R were made by using the QuickChange site-directed mutagenesis kit (Stratagene, La Jolla, Calif.). pcDNA6/His V5 rPGK1 contains non-sense mutations of C652G, T655C, C658G, and T661C. pcDNA6/His V5 rPDHK1 contains non-sense mutations of C848T, A850G, A853G, C854T, and T856G.

The pGIPZ control was generated with control oligonucleotide GCTTCTAACACCGGAGGTCTT (SEQ ID NO: 3). pGIPZ PGK1 shRNA and PDHK1 shRNA were generated with GGATGTCTATGTCAATGATGC (SEQ ID NO: 4) and CCGAACTAGAACTTGAAGA (SEQ ID NO: 5), respectively.

Purification of Recombinant Proteins.

WT and mutant His-PGK1, GST-PGK1, His-TOM20, His-PIN1, GST-PIN1, and SUMO-PDHK1 were expressed in bacteria and purified as described previously (Xia et al., 2007).

In Vitro Kinase Assays and Phosphoamino Acid Analysis (PAA).

The kinase reactions were performed as described previously (Fang et al., 2007). In brief, the bacterially purified recombinant PGK1 (10 ng) was incubated with PDHK1 (100 ng) in 25 μl of kinase buffer (50 mM Tris-HCl [pH 7.5], 100 mM KCl, 50 mM MgCl₂, 1 mM Na₃VO₄, 1 mM DTT, 5% glycerol, 0.5 mM ATP, and 10 ρCi [γ³²P]ATP) at 25° C. for 1 h. For the assay using 1,3-biphosphoglycerate (1,3-BPG) as a phosphate donor, active glyceraldehyde 3-phosphate dehydrogenase (200 ng) was incubated with PGK1 (100 ng) and PDHK1 (100 ng) in 25 μl reaction buffer (5 mM KH₂PO₄ [pH 7.0], 1 mM GAP, 0.3 mM beta-NAD, 5 mM MgSO₄, 100 mM glycine, 1 mM Na₃VO₄, 1 mM DTT) at 25° C. for 1 h. The reactions were terminated by the addition of SDS-PAGE loading buffer and heated to 100° C. The reaction mixtures were then subjected to SDS-PAGE analyses.

For PAA, the kinase reaction mixtures were separated by SDS-PAGE, transferred to polyvinylidene difluoride membrane, and the membrane corresponding to the mobility of phosphorylated PDHK1 was excised. PAA using two-dimensional electrophoresis on thin layer cellulose plates was performed as described previously (van der Geer and Hunter, 1994).

Immunofluorescence Analysis.

Immunofluorescence analyses were performed as described previously (Fang et al., 2007).

Molecular Modeling Analysis.

The molecular docking analysis of PGK1 and PIN1 proteins was performed using an online ZDOCK server (on the world wide web at zdock.umassmed.edu/). S203 of PGK1 and WW domain of PIN1 (K63 and R69) were chosen for docking.

Measurement of Pyruvate Oxidation Rate.

U87 cells (2×10⁶) cultured in a 25-cm² angled neck culture flask with a sodium bicarbonate free DMEM supplemented with 10% BCS, 20 mM HEPES, 5 mM glucose, and 1 mM pyruvate, were treated without or with hypoxia for 6 h, followed by adding 0.2 μCi of [1-¹⁴C]-pyruvate (American Radiolabeled Chemicals). The outlet of the flask was covered with filter paper soaked in hyamine hydroxide, and incubated at 37° C. for 2 h. The filter paper was removed, and the radioactivity was determined using a LS 6500 Multi-Purpose Scintillation Counter (Beckman Counter). The pyruvate oxidation rate was indicated as the radioactivity levels of samples normalized to cell numbers. For EGF treatment, 2×10⁶ U87/EGFR cells cultured in a 25-cm² angled neck culture flask with a sodium bicarbonate free DMEM supplemented with 0.5% BCS, 20 mM HEPES, 5 mM glucose, 1 mM pyruvate, were treated without or with EGF (100 ng/ml) for 6 h, followed by addition of 0.2 μCi of [1-¹⁴C]-pyruvate.

Measurement of Oxygen-Consumption Rate.

Cells (3×10⁴) were plated onto XF24 plates in DMEM (0.5% BCS, 25 mM glucose, 2 mM glutamine) (Seahorse Bioscience, North Billerica, Mass.) and incubated at 37° C., 5% CO₂ overnight, pretreated with 5 mM dichloroacetate for 1 h, and then stimulated with or without EGF (100 ng/ml) for 6 h. The medium was then replaced with 675 μl of unbuffered assay medium (Seahorse Bioscience) supplemented with 2 mM glutamine, 25 mM glucose (pH was adjusted to 7.4 using sodium hydroxide 0.5 mM). The cells were then placed at 37° C. in a CO₂-free incubator for 30 min. Basal oxygen-consumption rate (OCR) was recorded using the XF24 plate reader. Mitochondrial OCR was calculated (delta OCR value was the OCR difference of pre and post 1 μM rotenone treatment) and was normalized with cell numbers.

¹⁴C-Lipid Synthesis Assay.

¹⁴C-lipids derived from ¹⁴C-glucose or ¹⁴C glutamine were measured. Subconfluent cells seeded on a 6-well plate were pre-incubated with or without EGF (100 ng/ml) for 24 h. These cells were then incubated in fresh medium containing 1 μCi of D-[6-¹⁴C]-glucose (American Radiolabeled Chemicals) or L-[U-¹⁴C]-glutamine (PerkinElmer) for 2 h followed by PBS washing. Lipids were extracted by the addition of 500 μl hexane:isopropanol (3:2 v/v). The cells were incubated with an additional 500 μl of hexane:isopropanol solution. The lipid extracts were combined and air-dried with heat. Extracted lipids were resuspended in 50 μl chloroform and were subjected to scintillation counting. Scintillation counts were normalized with cell numbers.

Determining K_{M} of PGK1.

Purified recombinant WT PGK1 (0.2 ng) was incubated in 100 μl of reaction buffer (50 mM Tris-HCl [pH 7.5], 600 ng/μl Sumo-PDHK1, 5 mM MgSO₄, 1 mM Na₃VO₄, 1 mM DTT, 1.8 mM phosphorenolpyruvate, 7 units pyruvate kinase, 10 units lactic dehydrogenase, 0.01 mM NADH) with indicated ATP concentration at 37° C. in 96-well plate and were read by multidetection microplate readers (BMG LABTECH, Cary, N.C.) at 339 nm in kinetic mode for 5 min. The reaction velocity (V) was obtained by measuring the product concentration as a function of time. KM was calculated from a plot of 1/V vs. 1/[ATP] according to the Lineweaver-Burke plot model. Data represent the means ±SD of three independent experiments.

Measurement of ATP Concentrations in Mitochondrial Matrix.

Isolated mitochondria (2 mg) were washed in the washing buffer (200 mM sucrose, 10 mM HEPES [pH 7.4], 100 NM digitonin) and then lysed in 100 μl assay buffer. Samples were deproteinized using 10 kDa spin columns (Millipore), and ATP levels were determined using a BioVision ATP assay kit (BioVision).

Intracranial Injection.

GBM cells were intracranially injected (1×10⁶ cells in 5 μl of DMEM per mouse) with endogenous PGK1 or PDHK1 depletion and reconstituted expression of their WT or mutant proteins into 4-week-old female athymic nude mice. The injections were performed as described previously (Gomez-Manzano et al., 2006). Seven mice per group in each experiment were used. Animals injected with U87 or GSC11 cells were sacrificed 28 or 21 days after glioma cell injection, respectively. The brain of each mouse was harvested, fixed in 4% formaldehyde, and embedded in paraffin. Tumor formation and phenotype were determined by histologic analysis of H&E-stained sections. The animals were treated in accordance with relevant institutional and national guidelines and regulations. The use of animals was approved by the Institutional Review Board at The University of Texas MD Anderson Cancer Center.

Histologic Evaluation and Immunohistochemical Staining.

Mouse tumor tissues were fixed and prepared for staining. The specimens were stained with Mayer's hematoxylin and subsequently with eosin (H&E) (Biogenex Laboratories, San Ramon, Calif.). Afterward, the slides were mounted with use of Universal Mount (Research Genetics Huntsville, Ala.).

The tissue sections from paraffin-embedded human GBM specimens were stained with antibodies against phospho-PGK1 S203, phospho-PDHK1 T338, phospho-PDH S293, or nonspecific IgG as a negative control. The tissue sections were quantitatively scored according to the percentage of positive cells and staining intensity, as previously defined (Ji et al., 2009). The following proportion scores were assigned: 0 if 0% of the tumor cells showed positive staining, 1 if 0% to 1%, 2 if 2% to 10%, 3 if 11% to 30%, 4 if 31% to 70%, and 5 if 71% to 100%. The intensity of staining was rated on a scale of 0 to 3:0, negative; 1, weak; 2, moderate; and 3, strong. The proportion and intensity scores were combined to obtain a total score (range, 0-8), as described previously (Ji et al., 2009). Scores were compared with overall survival, defined as the time from date of diagnosis to death or last known date of follow-up. All patients had received standard adjuvant radiotherapy after surgery, which was followed by treatment with an alkylating agent (temozolomide in most cases). The use of human brain tumor specimens and the database was approved by the Institutional Review Board at The University of Texas MD Anderson Cancer Center.

TUNEL Assay.

Mouse tumor tissues were sectioned with 5 m thickness. Apoptotic cells were detected by using DeadEnd™ TUNEL Systems (Promega, Madison, Wis.) according to the manufacturer's instructions.

### Example 1—Hypoxia- and Activation of EGFR, K-Ras, and B-Raf-Induced Mitochondrial Translocation of PGK1 is Mediated by ERK1/2-Dependent PGK1 S203 Phosphorylation

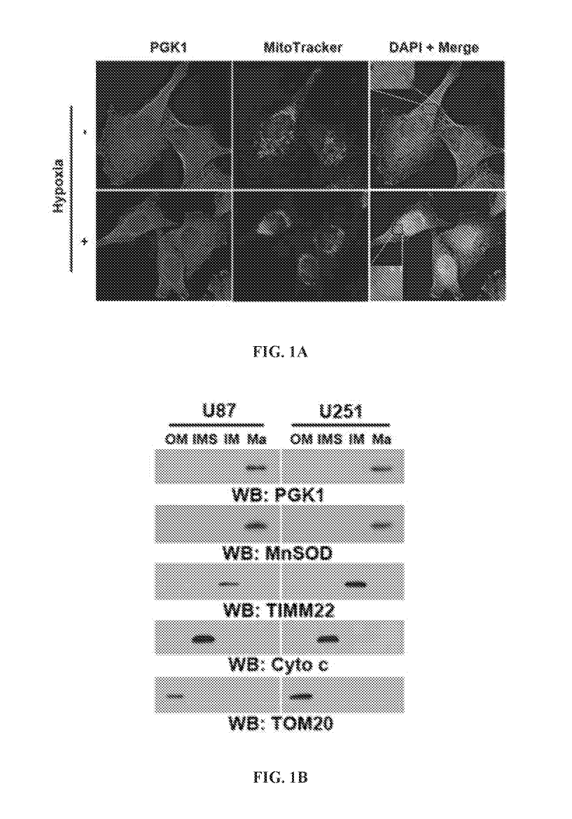

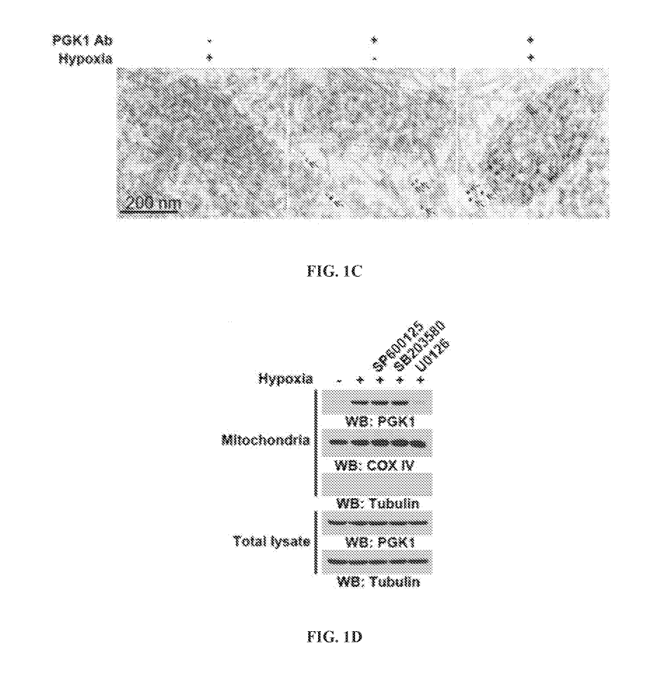

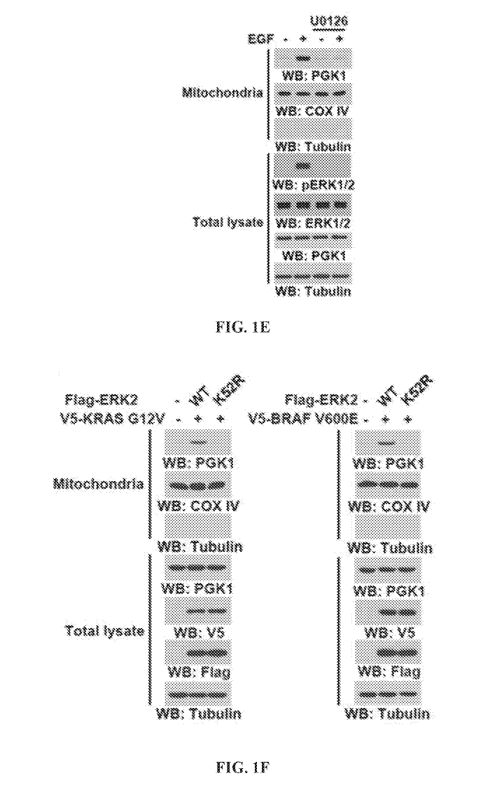

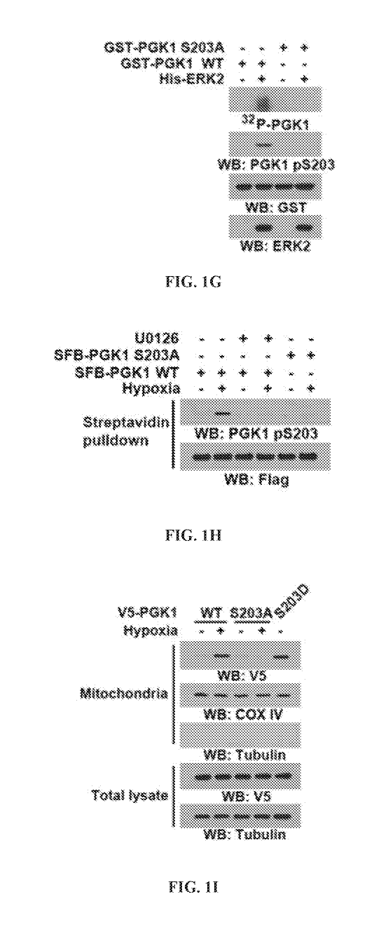

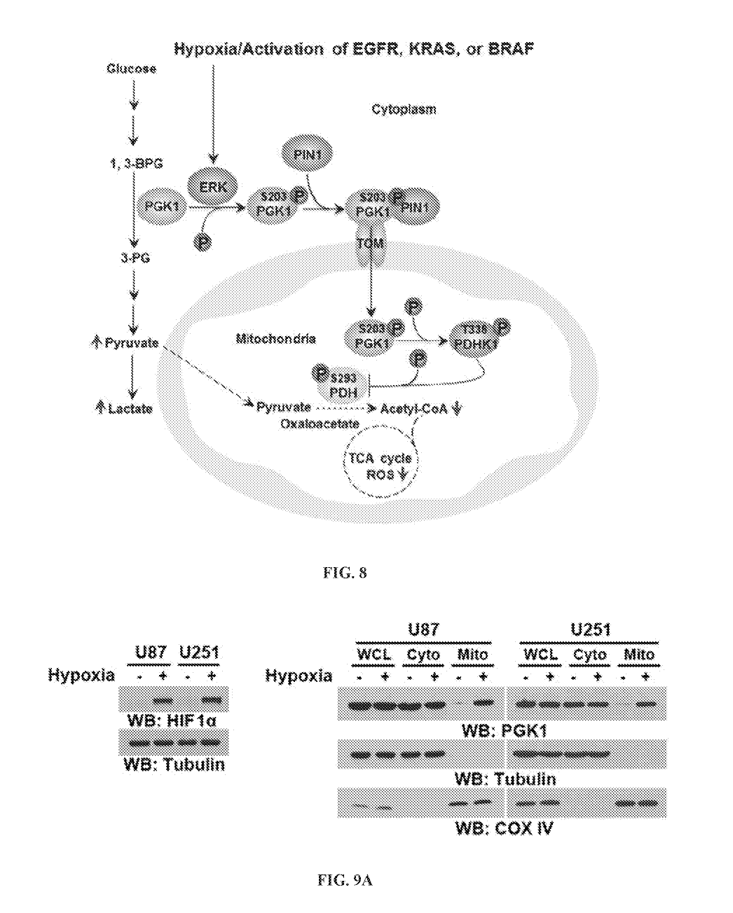

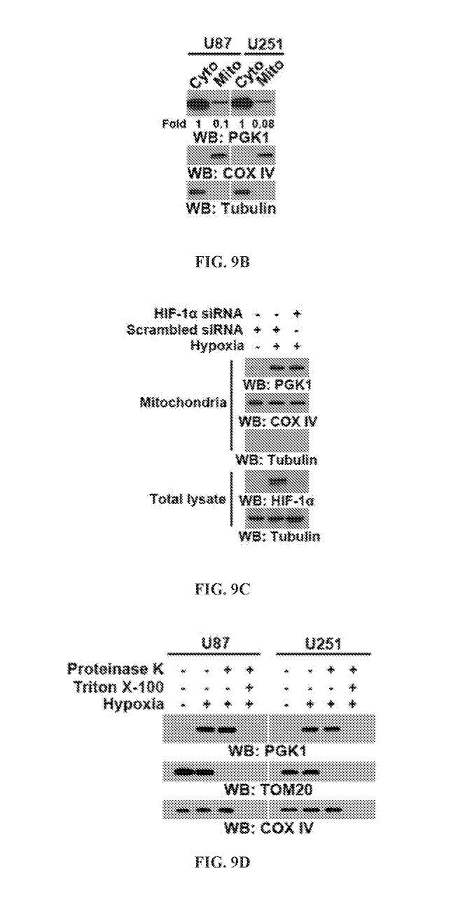

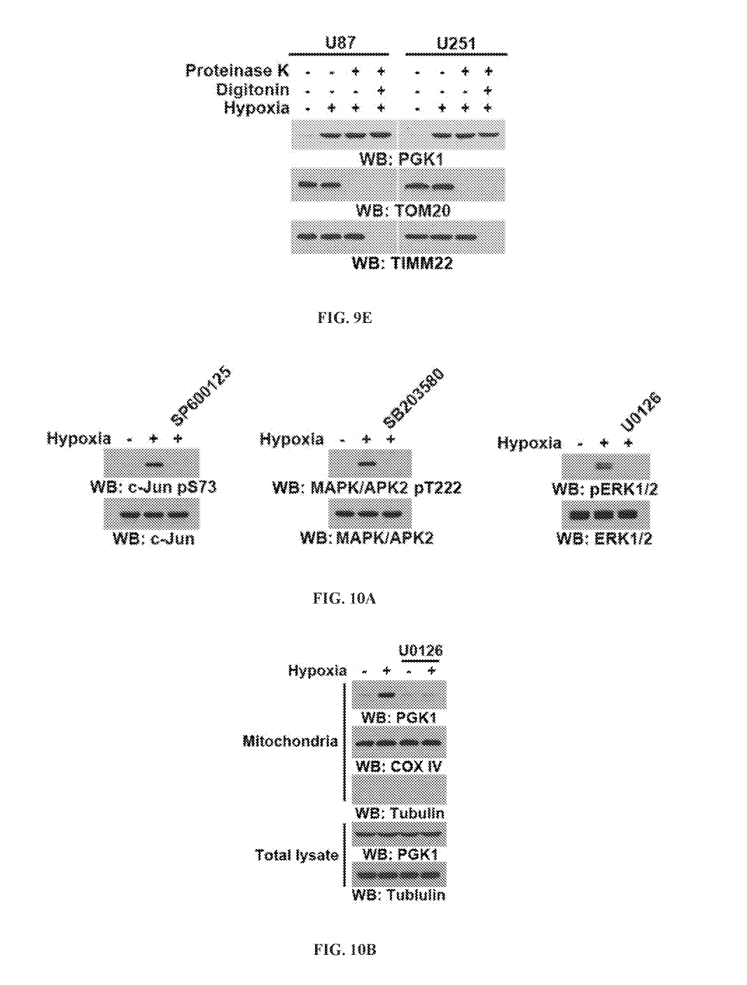

In solid tumors, hypoxia appears to be strongly associated with tumor growth and progression (Koppenol et al., 2011; Hockel and Vaupel, 2001; Caims et al., 2011). To test whether PGK1, an ATP-generating enzyme in the glycolytic pathway, has any subcellular compartment-dependent function, cells were exposed to hypoxia and the cellular distribution of PGK1 examined. U87 human glioblastoma (GBM) cells were incubated under hypoxic conditions for 6 h (FIG. 1A). Immunofluorescence analyses with an anti-PGK1 antibody showed that hypoxia induced the perinuclear accumulation of PGK1. Because mitochondria also localize at the perinuclear region, the cells were co-stained with the anti-PGK1 antibody and MitoTracker, a fluorescent mitochondrial dye. As shown in FIG. 1A, PGK1 co-localized with mitochondria, suggesting that PGK1 translocates to mitochondria upon hypoxic stimulation. This finding was supported by cell fractionation analyses that included mitochondrial marker COX IV and cytosolic tubulin as controls, showing that hypoxia, which resulted in HIF1α accumulation (FIG. 9A, left panel), induced mitochondrial translocation of PGK1 in both U87 and U251 GBM cells (FIG. 9A, right panel). Quantification analyses revealed that about 10% of cytosolic PGK1 translocates to mitochondria (FIG. 9B). Because prolonged hypoxic stimulation enhances PGK1 expression mediated by HIF1α (Marin-Hernandez et al., 2009; Semenza, 2010), whether PGK1 translocates to mitochondria in a HIF1α-dependent manner was next examined. Depletion of HIF1 a by siRNA did not block hypoxia-induced mitochondrial translocation of PGK1, indicating that this process occurs independently of HIF1α (FIG. 9C).

To determine whether PGK1 binds the outer membrane of mitochondria or translocates into them, a proteinase K protection assay was performed using mitochondria isolated from U87 and U251 cells with or without hypoxic stimulation. The outer membrane marker TOM20 and the intramitochondrial marker COX IV were included as controls (Hitosugi et al., 2011). In the absence of Triton X-100 (which solubilizes the outer and inner membranes of mitochondria), TOM20, but not PGK1 and COX IV, was completely digested by proteinase K treatment, whereas the presence of Triton X-100 made PGK1 and COX IV accessible to proteinase K digestion (FIG. 9D). In contrast, brief digitonin treatment, which damages the outer membrane, but not the inner membrane of mitochondria, had limited effect on the accessibility of mitochondrial PGK1 for proteinase K digestion (FIG. 9E). In addition, mitochondrial subfractionation analyses revealed that PGK1 was co-isolated with mitochondria matrix protein MnSOD, but not with inner membrane protein TIMM22 and intermembrane space protein cytochrome c (FIG. 1B), indicating that PGK1 translocated into the mitochondrial matrix. These findings were further supported by immunogold transmission electron microscopy analyses, which demonstrated that hypoxia induced the translocation of PGK1 into mitochondria (FIG. 1C).

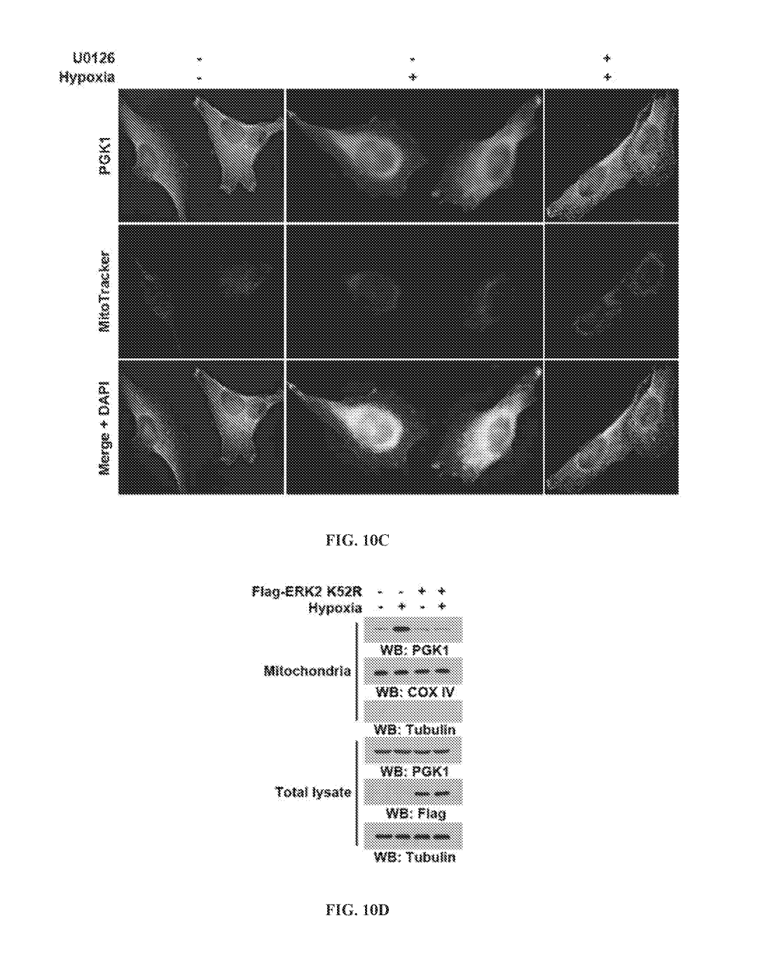

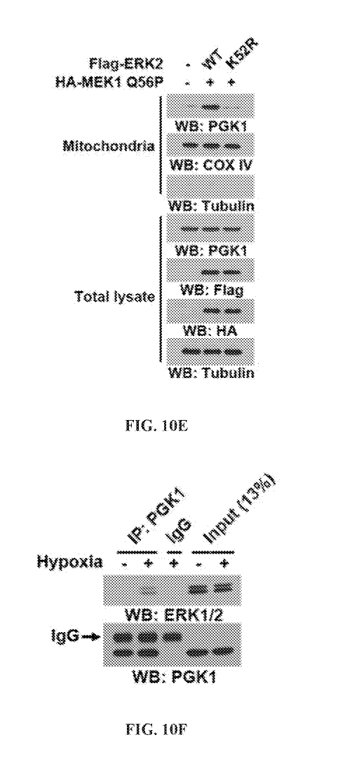

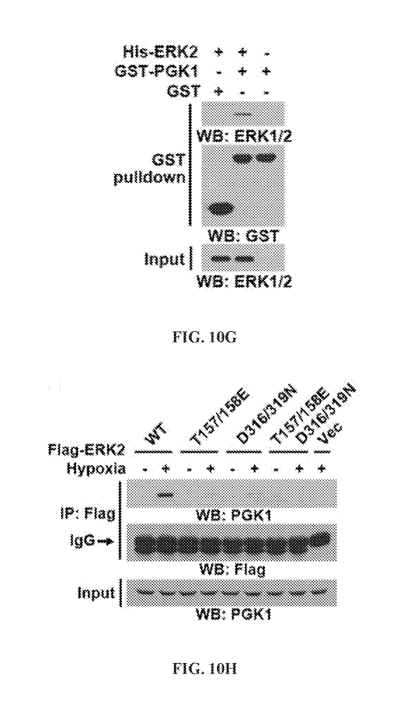

MAP kinase activation plays instrumental roles in hypoxia-induced cellular activities (Kronblad et al., 2005; Wykosky et al., 2011; Riley et al., 1986). Pretreatment of U87 cells with the JNK inhibitor SP600125, p38 inhibitor SB203580, or MEK/ERK inhibitor U0126 blocked hypoxia-induced phosphorylation of c-Jun, MAPK/APK2, and ERK1/2, respectively (FIG. 10A). Immunoblotting analyses revealed that only MEK/ERK inhibition notably reduced the hypoxia-induced mitochondrial translocation of PGK1 in U87 (FIG. 1D) and U251 cells (FIG. 10B). These results were supported by the results of immunofluorescence analyses (FIG. 10C). In addition, expression of the Flag-ERK2 K52R kinase-dead mutant blocked the hypoxia- (FIG. 10D) and active HA-MEK1 Q56P mutant-(FIG. 10E) induced mitochondrial translocation of PGK1. Thus, ERK1/2 activation is necessary and sufficient for mitochondrial translocation of PGK1. In line with this conclusion, EGF stimulation (FIG. 1E) or expression of oncogenic K-Ras G12V in BxPC-3 human pancreatic cancer cells (with no endogenous Ras mutation) and B-Raf V600E in CHL1 human melanoma cells (with no endogenous B-Raf mutation) (Flockhart et al., 2009; Yun et al., 2009) (FIG. 1F) induced mitochondrial translocation of PGK1; notably, this translocation was blocked by U0126 treatment or expression of ERK2 K52R kinase-dead mutant.

To examine whether ERK1/2 interacts with PGK1, an immunoblotting analysis of immunoprecipitated PGK1 was conducted with an anti-ERK1/2 antibody and showed that ERK1/2 associated with PGK1 upon hypoxia stimulation (FIG. 10F). In addition, an in vitro GST pull-down assay with incubation of purified recombinant active His-ERK2 with GST-PGK1 revealed that these two proteins directly interact with each other (FIG. 10G).

The docking groove of the MAP kinases, which consists of the common docking (CD) domain and glutamate/aspartate (ED) sites, serves as a common docking region for various MAP kinase-interacting molecules (Lu and Xu, 2006). D316 and D319 in the CD domain and T157 and T158 in the ED sites of ERK2 are important for the recognition of its substrates (Lu and Xu, 2006). Co-immunoprecipitation assays revealed that binding of endogenous PGK1 to Flag-ERK2 D316/D319N and to Flag-ERK2 T157/T158E was greatly reduced, and completely failed to interact with an ERK2 mutant with combined mutations of the CD domain and ED sites (FIG. 10H). These results indicate that PGK1 binds to the ERK2 docking groove.

ERK substrates often have a docking domain that is characterized by a cluster of basic residues followed by an LXL motif (L represents leucine, but can also be isoleucine or valine; X represents any amino acid) (Yang et al., 2012). Analysis of the PGK1 amino acid sequence with the Scansite program identified the putative ERK-binding sequences 74-DKYSLEPVAVE-84 (SEQ ID NO: 6), 170-HRAHSSMVGVN-180 (SEQ ID NO: 7), and 273-AEKNGVKITLP-283 (SEQ ID NO: 8), which contain LXL motifs at V81/V83, V177/V179, and V278/I280/L282, respectively. Streptavidin pull-down of S-Flag-Biotin (SFB)-PGK1 proteins showed that only PGK1 V278A/I280R/L282R mutation markedly reduced its binding to ERK1/2 (FIG. 10I). These results indicate that the ERK2 docking groove binds to a docking domain in PGK1 at V278/I280/L282.

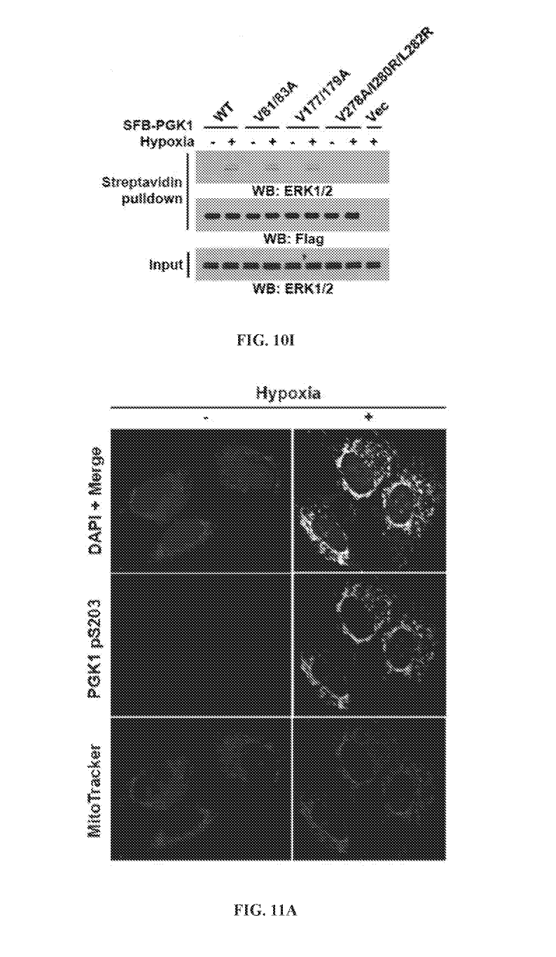

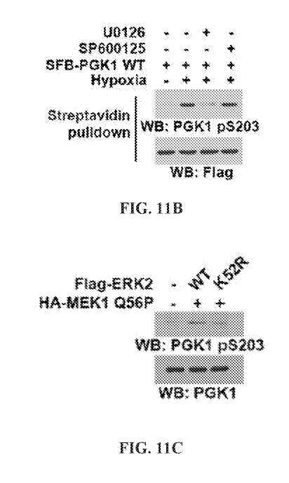

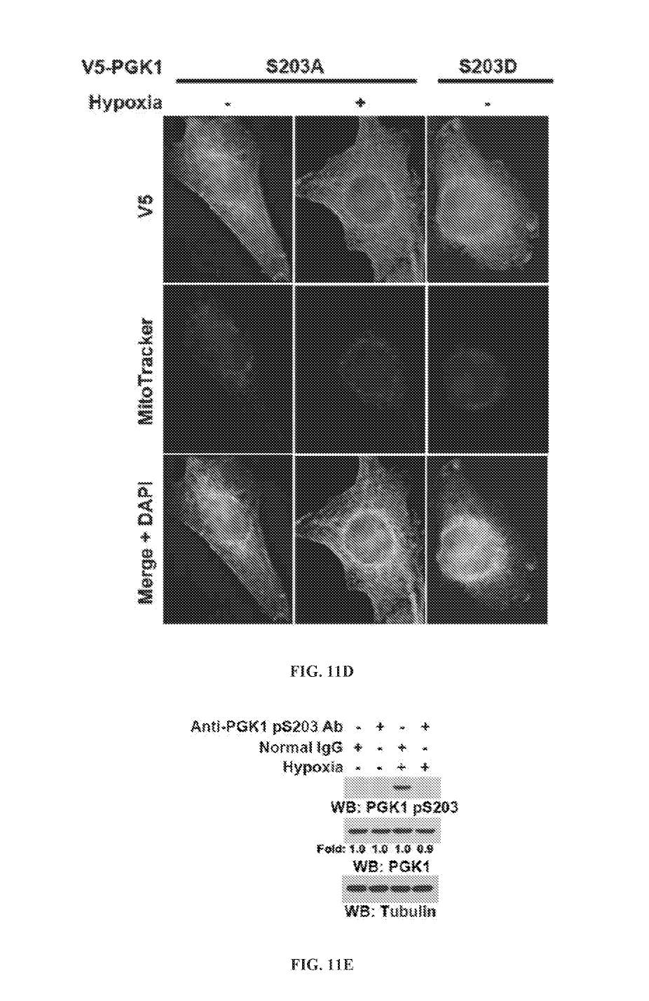

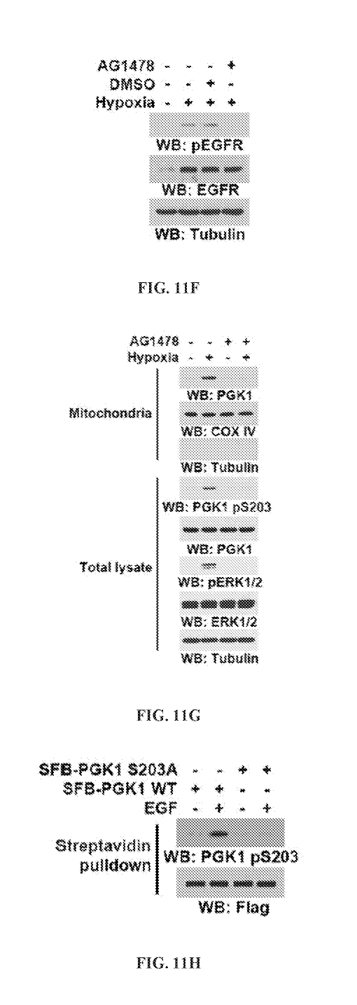

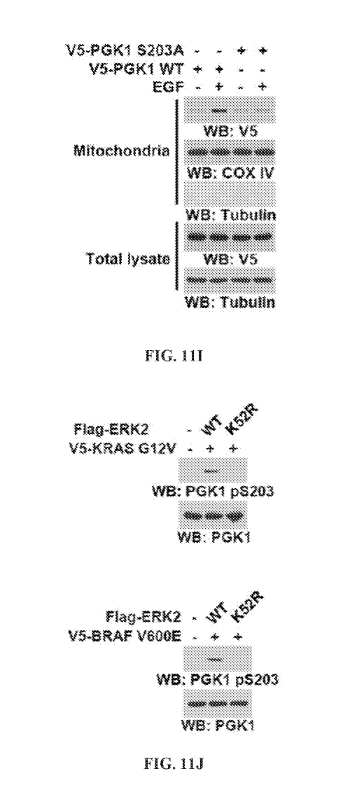

To determine whether PGK1 is a substrate of ERK1/2, an in vitro kinase assay was performed showing that purified and active ERK2 phosphorylated bacterially purified PGK1 (FIG. 1G). ERK1/2 phosphorylates its substrates with a P-X-S/TP (where X can be any amino acid) consensus sequence (Ji et al., 2009). An analysis of PGK1 amino acid sequences revealed that PGK1 has only one ERK1/2 phosphorylation motif, which is at S203. Mutation of S203 to Ala abolished ERK2-mediated PGK1 phosphorylation in vitro with [γ³²P]-ATP, as demonstrated by an autoradiography assay and immunoblotting analyses using a specific anti-PGK1 pS203 antibody (FIG. 1G). Immunofluorescence analysis showed that hypoxia stimulation resulted in accumulation of phosphorylated PGK1 S203 in mitochondria (FIG. 11A). In addition, pull-down of SFB-tagged wild-type (WT) PGK1 and PGK1 S203A by streptavidin agarose beads revealed that U0126 treatment and PGK1 S203A mutation (FIG. 1H), but not SP600125 treatment (FIG. 11B), abolished hypoxia-induced PGK1 S203 phosphorylation in U87 cells. In line with these findings, expression of ERK2 K52R kinase-dead mutant blocked active MEK1 Q56P-induced PGK1 S203 phosphorylation (FIG. 11C). Notably, PGK1 S203A was resistant to hypoxia-induced mitochondrial translocation (FIGS. 11 and 11D). In contrast, a phosphorylation-mimic PGK1 S203D mutant was able to accumulate in mitochondria in the absence of hypoxia stimulation (FIGS. 11 and 11D), indicating that ERK1/2-mediated PGK1 phosphorylation at S203 is required for mitochondrial translocation of PGK1. Quantification of cytosolic PGK1 with or without immune-depletion using an anti-phospho-PGK1 S203 antibody showed that depletion of phosphorylated PGK1 only removed about 10% of total PGK1 protein (FIG. 11E), further supporting the finding that only a small portion of PGK1 translocated into mitochondria.

Hypoxia resulted in EGFR activation (Michelson et al., 1985). Treatment with EGFR inhibitor AG1478 blocked hypoxia-induced EGFR phosphorylation (FIG. 11F), ERK activation, and phosphorylation and mitochondrial translocation of PGK1 (FIG. 11G). These results indicated that hypoxia induces ERK activation and mitochondrial translocation of PGK1 through activation of EGFR. Consistent with this finding, EGF-induced PGK1 S203 phosphorylation (FIG. 11H) and S203 phosphorylation-dependent mitochondrial translocation of PGK1 (FIG. 11I) were also observed. In addition, expression of ERK K52R blocked K-Ras G12V- and B-Raf V600E-induced PGK1 S203 phosphorylation (FIG. 11J). These findings indicated that hypoxia, activation of EGFR, and expression of oncogenic K-Ras and B-Raf induces ERK-dependent phosphorylation and mitochondrial translocation of PGK1.

### Example 2—PIN1 Binds to and Cis-Trans Isomerizes Phosphorylated PGK1 for Mitochondrial Translocation of PGK1

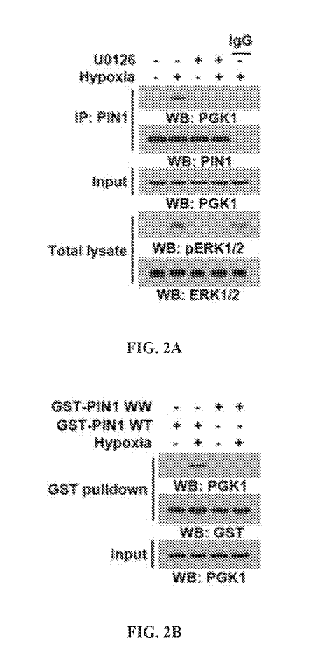

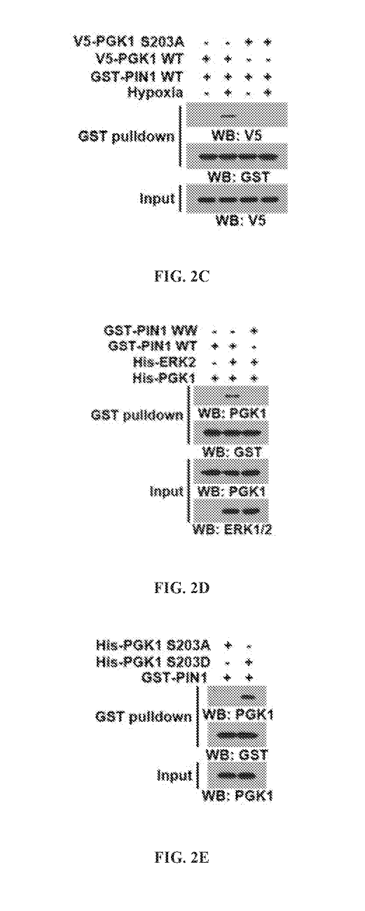

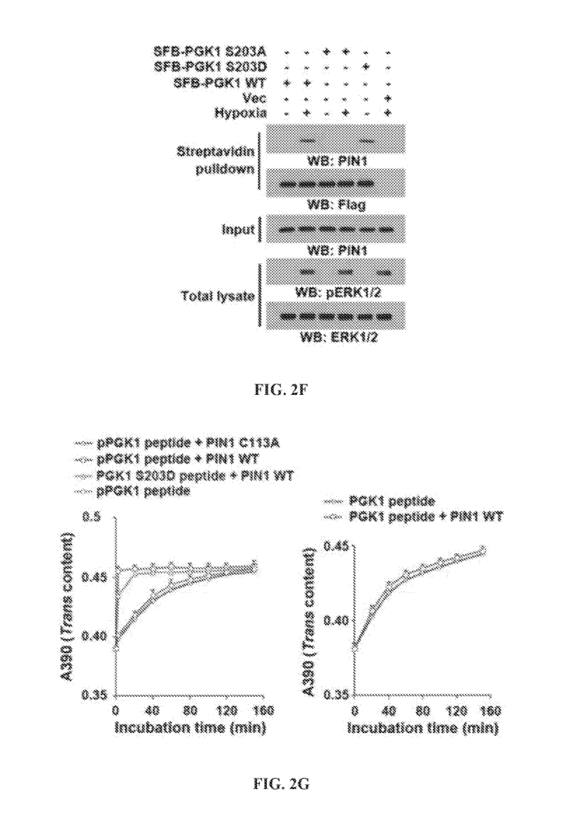

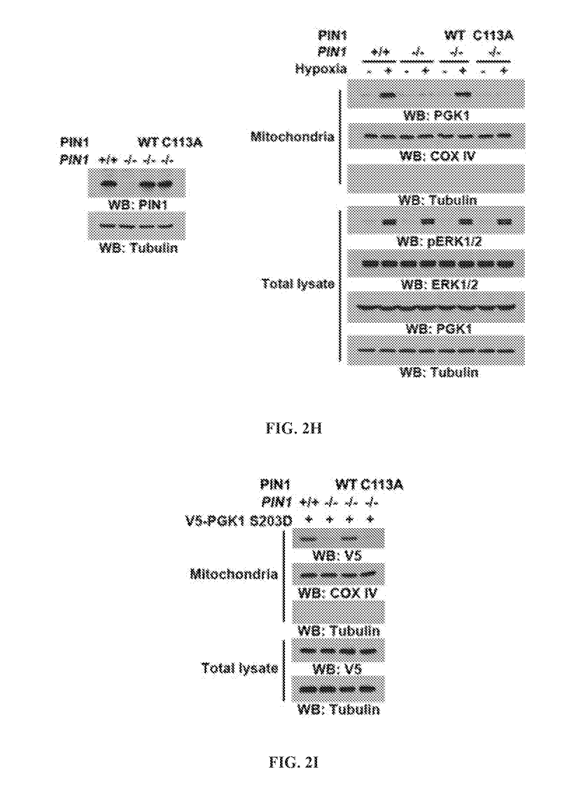

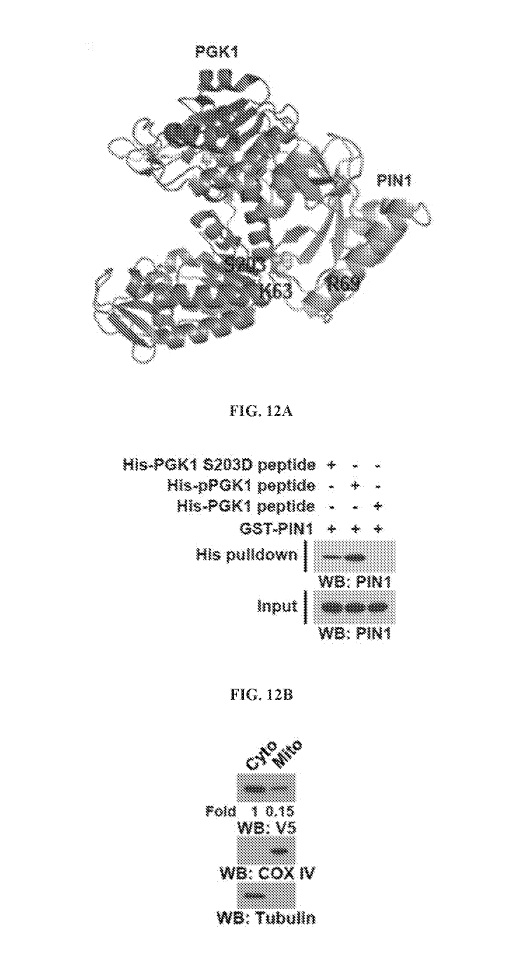

ERK-phosphorylated Ser or Thr in pS/TP-peptide sequences can be recognized by the peptidylproline isomerase Protein Interacting with Never in Mitosis A 1 (PIN1), which catalyzes their cis-trans isomerization (Lu and Zhou, 2007; Zheng et al., 2009). PIN1 regulates subcellular redistribution of its substrates (Yang et al., 2012). Whether ERK-regulated PGK1 phosphorylation leads to PIN1-dependent conformational change of PGK1 was determined and subsequent mitochondria translocation of PGK1. Coimmunoprecipitation analyses showed that hypoxia stimulation significantly increased the interaction between endogenous PIN1 and PGK1, which was blocked by U0126 treatment (FIG. 2A). In addition, hypoxia stimulation induced a strong binding of endogenous PGK1 to agarose bead-immobilized WT GST-PIN1 but not GST-PIN1 WW domain mutant, which prevents the binding of PIN1 to a pS/TP substrate (FIG. 2B). Compared with its WT counterpart, PGK1 S203A failed to interact with GST-PIN1 (FIG. 2C). Molecular modeling analysis of PGK1 and PIN1 protein structures showed that S203 of PGK1 can be adjacent to the K63 and R69 phosphobinding pocket in the WW domain (FIG. 12A) (Michelson et al., 1985; Yaffe et al., 1997), suggesting that the PIN1 WW domain is able to bind to phosphorylated S203P. The requirement of ERK1/2 for the interaction between PIN1 and PGK1 was confirmed by an in vitro binding assay, which showed that purified His-PGK1 binds to purified WT GST-PIN1 but not GST-PIN1 WW mutant, only in the presence of ERK2 and ATP (FIG. 2D). In addition, purified PGK1 S203D, but not PGK1 S203A, was able to pull down either purified GST-PIN1 (FIG. 2E) or endogenous PIN1 in U87 cells without hypoxia stimulation (FIG. 2F).

To further examine whether the phosphorylated S203/P204 motif of PGK1 is a PIN1 substrate, PGK1 oligopeptides were synthesized containing phosphorylated or nonphosphorylated S203/P204. WT GST-PIN1, but not a catalytically inactive GST-PIN1 C113A mutant, isomerized the phosphorylated S203/P204 peptide (FIG. 2G, left panel) but not its nonphosphorylated counterpart (FIG. 2G, right panel). In addition, GST-PIN1 isomerized the phosphorylation-mimic D203/P204 peptide, which occurred at a lower efficiency than for phosphorylated S203/P204 peptide, but at a higher efficiency than for the nonphosphorylated peptide (FIG. 2G, left panel). Consistent with this finding, His-tagged phosphorylation-mimic D203/P204 peptide bound to GST-PIN1 with a lower efficiency than the phosphorylated peptide, but with a higher affinity than its nonphosphorylated counterpart (FIG. 12B). Cell fraction analysis showed that about 15% of PGK1 S203D translocated into the mitochondria (FIG. 12C), which may reflect the low efficiency of PGK1 S203D.P204 isomerization by PIN1 and the limited amount of available PIN1 for PGK1 binding. These results suggest that PIN1 specifically isomerizes the phosphorylated S203/P204 within PGK1.

To determine the role of PIN1 in the mitochondrial translocation of PGK1, hypoxia was used to stimulate PIN1^{+/+}, PIN1^{+/-}, or PIN1^{-/-} mouse embryonic fibroblasts with reconstituted expression of WT PIN1 or a PIN1 C113A mutant (FIG. 2H, left panel). PIN1 deficiency completely blocked the hypoxia-induced mitochondrial translocation of PGK1, whereas this block was rescued by re-expression of WT PIN1 but not the PIN1 C113A mutant (FIG. 2H, right panel). In addition, PIN1 deficiency blocked the mitochondrial translocation of the phosphorylation-mimic PGK1 S203D mutant, and the failure of PGK1 S203D translocation was rescued by reconstituted expression of WT PIN1 but not the PIN1 C113A mutant (FIG. 2I). These results indicate that ERK1/2-mediated PGK1 phosphorylation leads to the binding of PIN1 to PGK1, which in turn leads to the cis-trans isomerization and subsequent mitochondrial translocation of PGK1.

### Example 3—PIN1 Regulates Binding of PGK1 to the TOM Complex

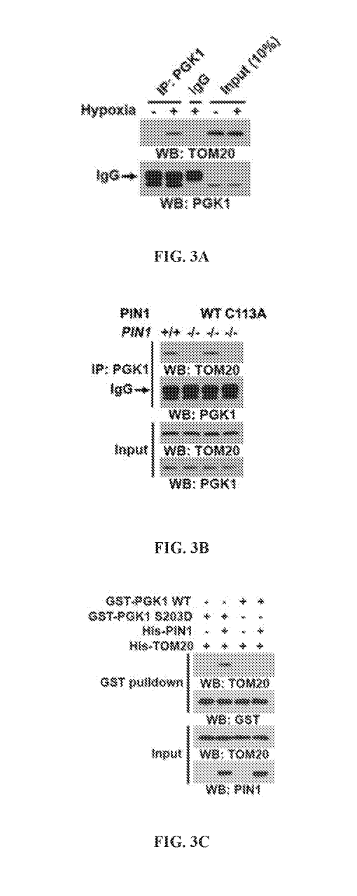

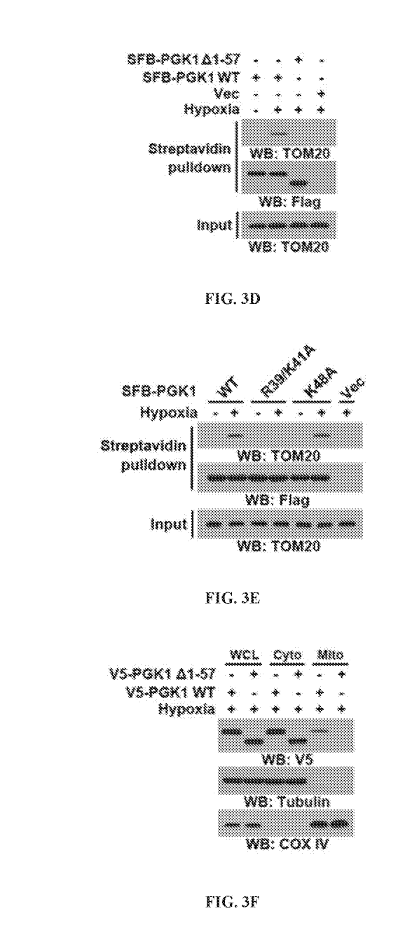

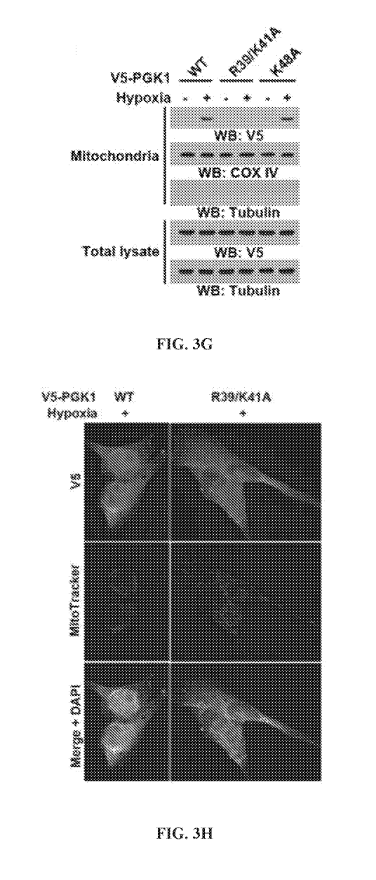

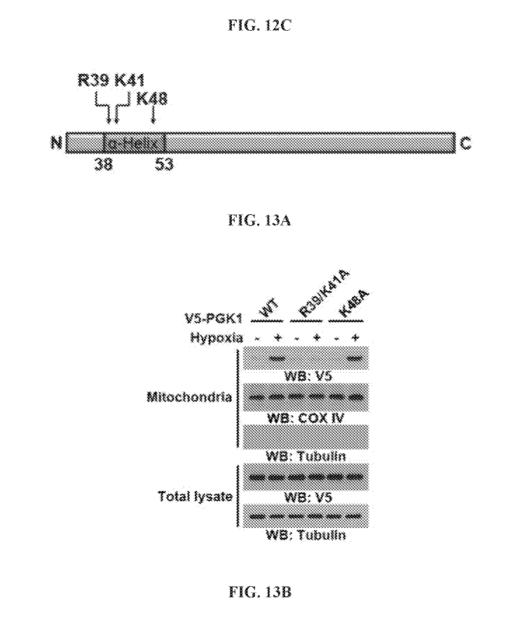

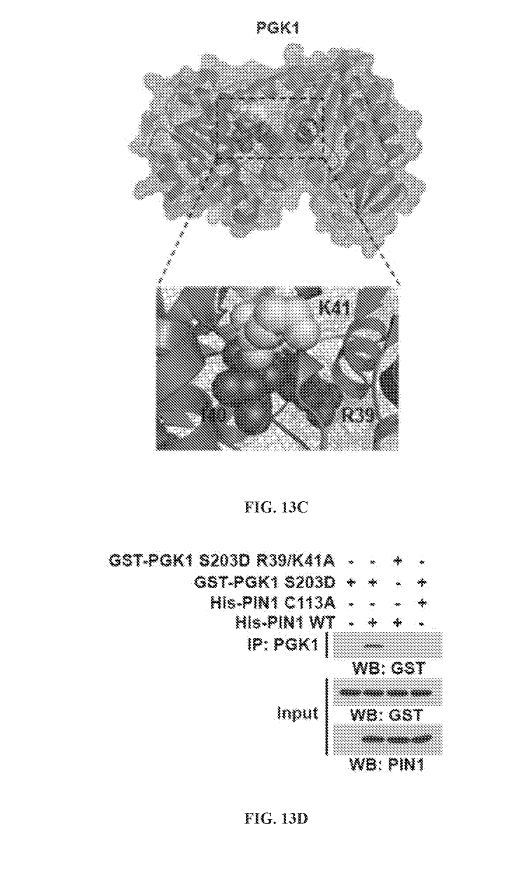

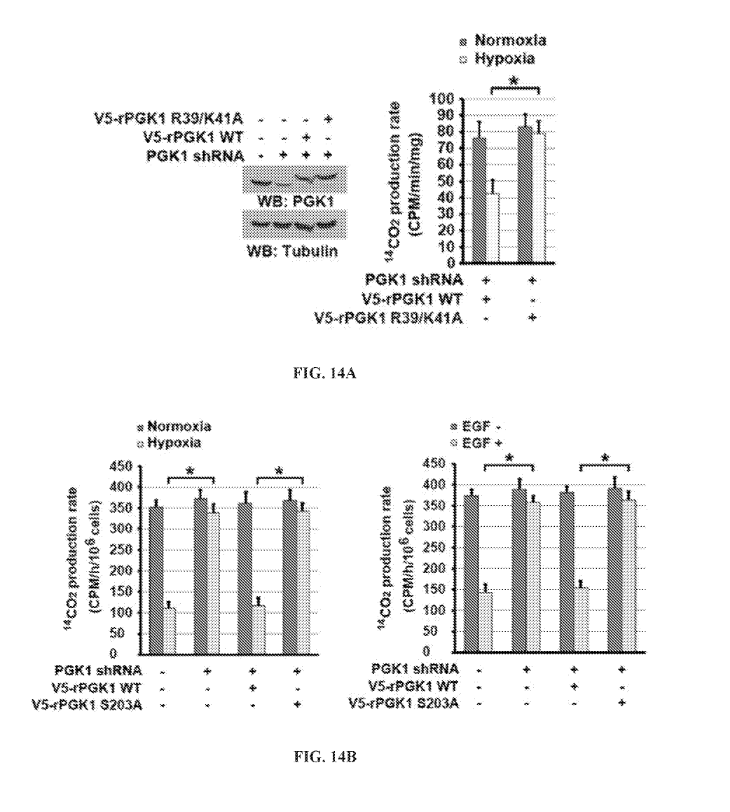

Nearly all mitochondrial pre-proteins are imported via the general entry gate, which is the translocase of the outer membrane (TOM). Three receptor proteins, TOM20, TOM70, and TOM22, function as part of the TOM complex. TOM20 acts as a general import receptor and is the initial recognition site for substrates with presequences (Chacinska et al., 2009). Presequences, which are often located at the amino terminus of precursor proteins and form positively charged amphipathic a helices, are the classic type of mitochondrial targeting signals (Chacinska et al., 2009). A structural analysis of PGK1 revealed that it contains an a-helix (amino acids 38-53) at its N-terminus (FIG. 13A). Co-immunoprecipitation analyses showed that hypoxia stimulation resulted in an interaction between PGK1 and TOM20 (FIG. 3A). PIN1 deficiency abrogated this interaction, which was rescued by reconstituted expression of WT PIN1 but not the PIN1 C113A mutant (FIG. 3B). In addition, incubation of purified WT GST-PGK1 or GST-PGK1 S203D mutant with purified His-TOM20 in the presence or absence of PIN1 showed that GST-PGK1 S203D, but not WT GST-PGK1, was able to bind to TOM20 in the presence, but not absence, of PIN1 (FIG. 3C). These results indicate that PIN1 is required for phosphorylated PGK1 to bind to the TOM complex.

To determine the presequence of PGK1 needed for its mitochondrial translocation, the N-terminal 1-57 amino acids containing an a-helix were deleted and the protein expressed as a C-terminally SFB-tagged protein in U87 cells. SFB-PGK1 Δ1-57, unlike its WT counterpart, lost the ability to interact with TOM20 (FIG. 3D). Mutation of positively charged R39, K41, and K48 in the a-helix into Ala showed that PGK1 R39/K41A, but not PGK1 K48A, abrogated the interaction between PGK1 and TOM20 (FIG. 3E), strongly suggesting that R39/K41 are the residues of PGK1 involved in binding to the TOM complex. Notably, neither the PGK1 Δ1-57 mutant (FIG. 3F) nor the PGK1 R39/K41A mutant (FIGS. 3G-H) was able to translocate into mitochondria upon hypoxia stimulation of U87 cells, as demonstrated by immunoblotting (FIGS. 3F-G) and immunofluorescence (FIG. 3H) analyses. Similar results for PGK1 R39/K41A were observed in U251 cells (FIG. 13B). Given that PGK1 R39/K41 are not exposed on the surface of the PGK1 protein structure (FIG. 13C), these results strongly suggest that PIN1-dependent cis-trans isomerization of the pS203.P204 bond in PGK1 exposes the mitochondrial targeting signal containing R39/K41 residues of PGK1 for recognition by the TOM complex. To further support this conclusion, purified GST-PGK1 S203D mutant was incubated with or without purified PIN1 for isomerization, which was followed by immunoprecipitation with the specific anti-PGK1 antibody that recognizes 38-QRIKAA-43 (SEQ ID NO: 9). Immunoblotting analyses with an anti-GST antibody showed that anti-PGK1 antibody against 38-QRIKAA-43 successfully recognized GST-PGK1 S203D in the presence of PIN1, but not in the absence of PIN1 (FIG. 13D). These results suggested that PIN1-regulated isomerization of PGK1 exposes the 38-QRIKAA-43 residues so that they can be recognized by the peptide-specific antibody.

### Example 4—Mitochondrial PGK1 Phosphorylates PDHK1

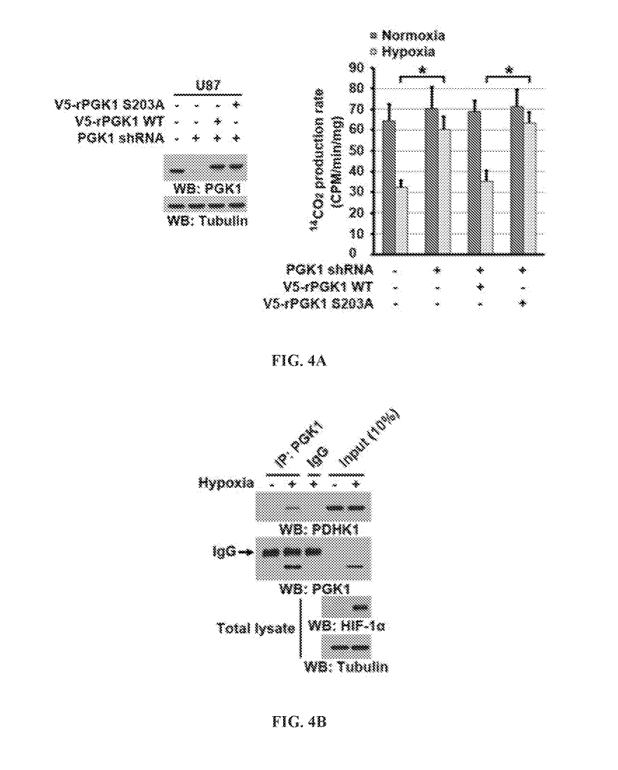

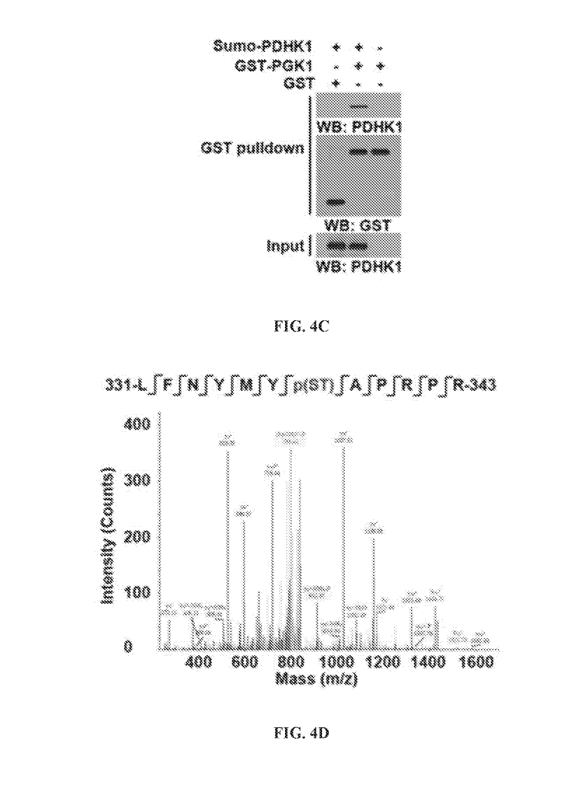

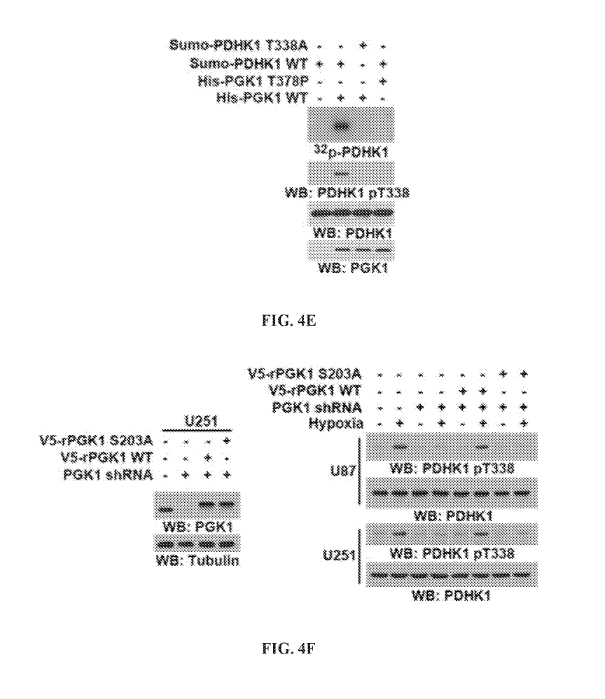

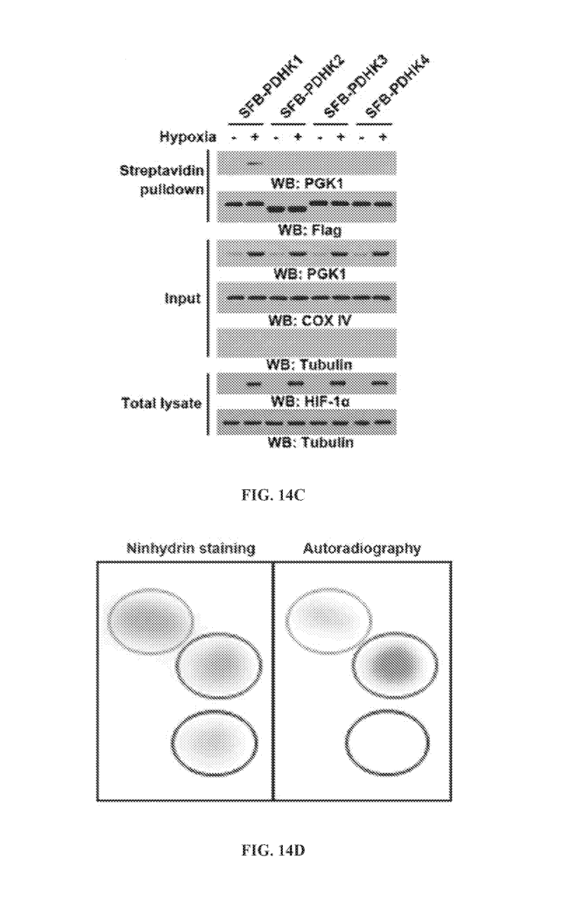

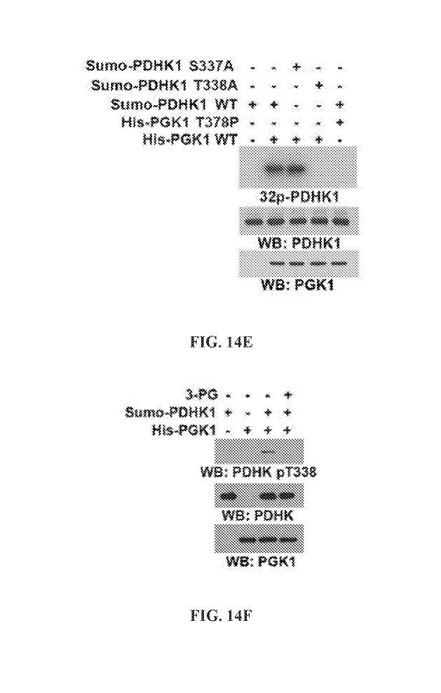

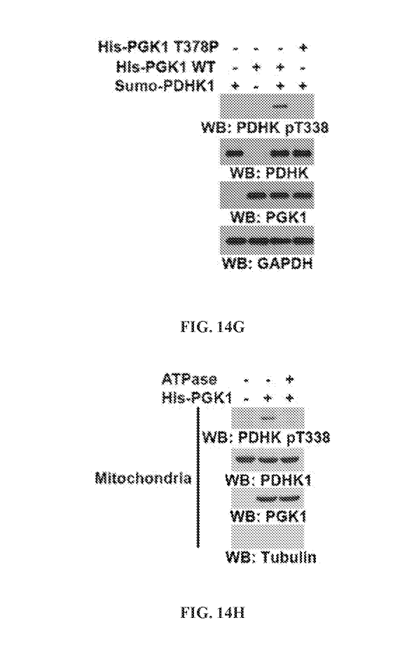

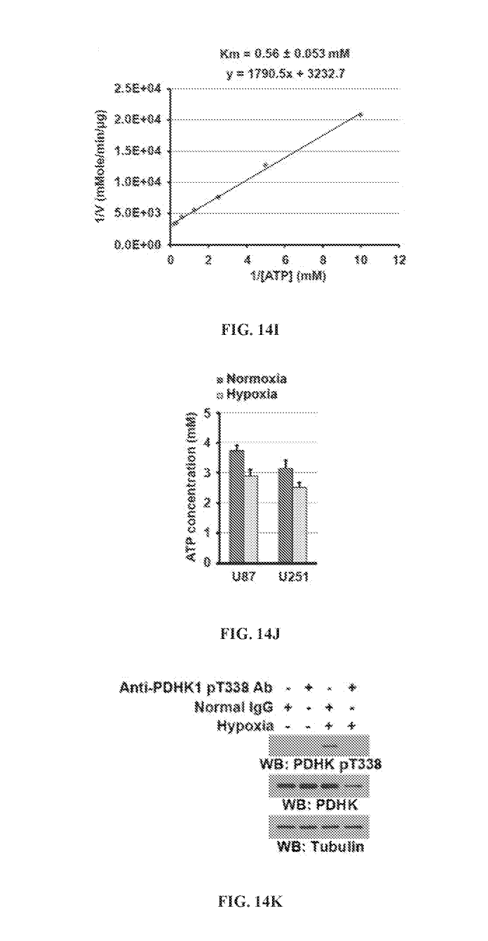

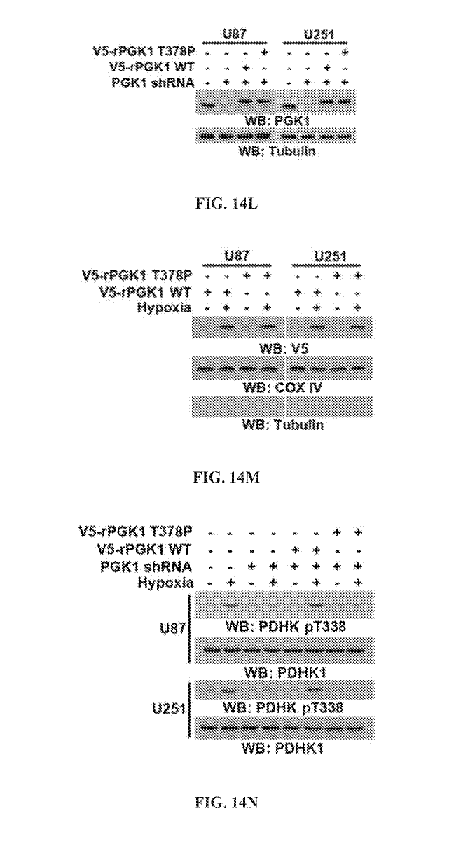

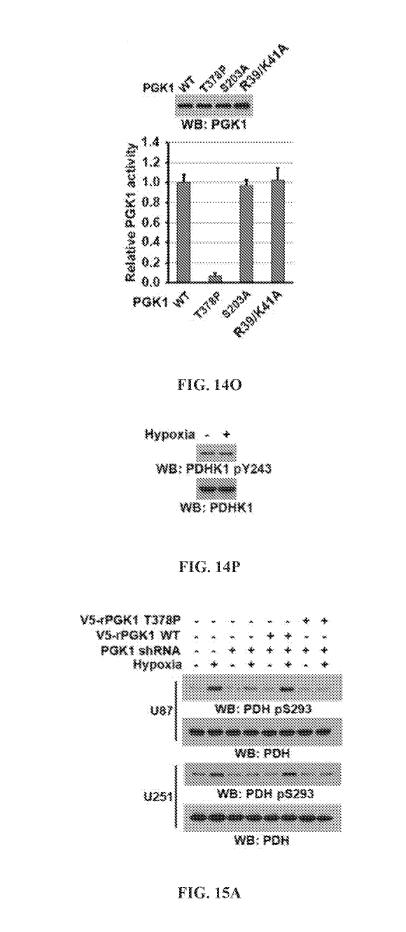

Hypoxia enhances the glycolytic pathway and results in pyruvate being converted into lactate rather than being used for mitochondrial oxidation (Vander Heiden et al., 2009; Semenza, 2010). As expected, hypoxia decreased the conversion rate of ¹⁴C-labeled pyruvate to ¹⁴C-labeled CO₂ in isolated mitochondria (FIG. 4A). To determine whether mitochondrial PGK1 regulates the rate of PDH complex-mediated conversion of pyruvate to CO₂, PGK1 was depleted with short hairpin RNA (shRNA) and the expression of RNA interference-resistant (r) V5-tagged WT PGK1, rPGK1 S203A, or rPGK1 R39/K41A was reconstituted in U87 and U251 cells. PGK1 depletion significantly counteracted the suppression of hypoxia-induced conversion of pyruvate to CO₂, and this suppression was rescued by reconstituted expression of WT rPGK1, but not of rPGK1 S203A or rPGK1 R39/K41A, in U87 cells (FIGS. 4A and 14A). Similar results were also obtained for hypoxia- or EGF-stimulated cells incubated with [1-¹⁴C]-pyruvate (FIG. 14B). These results indicate that mitochondrial PGK1 regulates the activity of the PDH complex.

In line with this finding, immunoblotting analyses of immunoprecipitated PGK1 from mitochondria of U87 cells with an anti-PDHK1 antibody showed that hypoxia induced an interaction between endogenous PGK1 and PDHK1 (FIG. 4B). In addition, a Streptavidin pulldown assay showed that SFB-tagged PDHK1, but not PDHK2, PDHK3, or PDHK4, interacted with endogenous PGK1 upon hypoxic stimulation (FIG. 14C). Furthermore, an in vitro binding assay in which bacterially purified SUMO-tagged PDHK1 proteins were mixed with purified and immobilized GST or GST-PGK1 showed that PGK1 directly bound to PDHK1 (FIG. 4C). These results indicated that hypoxia results in direct interaction between PGK1 and PDHK1.

PKM2 and PGK1 catalyze the only two ATP-generating reactions in the glycolytic pathway. PGK1-catalyzed conversion of 1,3-BPG to 3-PG and ATP is a reversible reaction (Bernstein and Hol, 1998) such that PGK1 can also utilize ATP as a phosphate donor. PKM2 functions as a protein kinase (Yang et al., 2012). To test whether PGK1 might act as a protein kinase to phosphorylate PDHK1, an in vitro phosphorylation assay was performed by mixing bacterially purified PGK1, PDHK1, and ATP. Liquid chromatography-coupled Orbitrap mass spectrometry (LC-MS/MS) analyses of tryptic digests of PDHK1 showed that PGK1 phosphorylates PDHK1 at S337 or T338 (FIG. 4D). Phosphoamino acid analysis with [γ³²P]-ATP showed that PGK1 phosphorylates PDHK1 predominantly at threonine (FIG. 14D), suggesting that PDHK1 T338 is phosphorylated. In addition, WT PGK1, but not a PGK1 T378P kinase-dead mutant (Chiarelli et al., 2012), was able to phosphorylate WT PDHK1, but not PDHK1 T338A, in the presence of [γ³²P]-ATP, which was detected by both autoradiography and a specific anti-phospho-PDHK1 T338 antibody (FIG. 4E). In contrast, mutation of the adjacent PDHK1 S337A had no effect on PGK1-mediated PDHK1 phosphorylation (FIG. 14E). This phosphorylation was abrogated by incubation with an excess amount of 3-PG (FIG. 14F), suggesting that PDHK1 and 3-PG compete with each other for phosphorylation by PGK1. In addition, PDHK1 T338 was phosphorylated upon incubation of purified PGK1, PDHK1, and 1,3-BPG, which was generated by incubation of glyceraldehyde phosphate dehydrogenase (GAPDH) with glyceraldehyde 3-phosphate and NAD⁺ (FIG. 14G). These results indicated that both ATP and 1,3-BPG can be a phosphate donor for PDHK1 phosphorylation in vitro.

To identify the physiological phosphate donor for PGK1-mediated PDHK1 phosphorylation, mitochondria were extracted and the mitochondrial lysate mixed with purified PGK1 in the presence or absence of exogenous ATPase. As demonstrated in FIG. 14H, incubation with ATPase, which hydrolyzes mitochondrial ATP, abrogated PGK1-mediated PDHK1 T338 phosphorylation. These results strongly suggested that PGK1 utilizes ATP as a phosphate donor in mitochondria to phosphorylate PDHK1. Given that the Km (0.56±0.053 mM) of ATP for PGK1-dependent PDHK phosphorylation (FIG. 14I) is much lower than the physiological mitochondrial concentrations of ATP in U87 and U251 cells, which range from 2.5 to 3.5 mM under normoxic and hypoxic conditions (FIG. 14J), these results suggest that PGK1 is able to efficiently phosphorylate PDHK1 in mitochondria utilizing ATP.

Depletion of phosphorylated PDHK1 from a mitochondrial extract using a PDHK1 pT338 antibody largely reduced the amount of PDHK1 in mitochondria, suggesting that the majority of PDHK1 was phosphorylated by PGK1 in mitochondria upon hypoxic stimulation (FIG. 14K). To determine whether PGK1 phosphorylates PDHK1 in cells, PGK1 expression was depleted in U87 and U251 cells with PGK1 shRNA, with or without reconstituted expression of WT rPGK1 or the rPGK1 T378P catalytically-dead mutant (FIG. 14L). Mitochondrial fraction analyses showed that both WT rPGK1 and the inactive mutant of rPGK1 were able to translocate into mitochondria (FIG. 14M). Immunoblotting analyses showed that PGK1 depletion blocked hypoxia-induced PDHK1 T338 phosphorylation, which was rescued by reconstituted expression of WT rPGK1 but not rPGK1 T378P (FIG. 14N). In addition, reconstituted expression of rPGK1 S203A, which had comparable glycolytic activity to its WT counterpart (FIG. 14O), failed to induce PDHK1 T338 phosphorylation under hypoxic conditions (FIG. 4F). PDHK1 is known be phosphorylated at tyrosine residues by FOP2-fibroblast growth factor receptor (FGFR) 1, an oncogenic, soluble FGFR1 fusion protein (Hitosugi et al., 2011). Consistent with this previous finding (Hitosugi et al., 2011), PDHK1 Y243 phosphorylation mediated by activated FOP2-FGFR was not altered upon hypoxic stimulation (FIG. 14P). Given that depletion of phosphorylated PDHK1 in mitochondria with a PDHK1 pT338 antibody largely reduced the amount of PDHK1 in mitochondria, these results suggest that FGFR is not involved in the regulation of PDHK1 during hypoxia. These results indicated that PGK1 functions as a protein kinase and phosphorylates PDHK1 T338 in mitochondria under hypoxic condition.

### Example 5—PDHK1 Phosphorylation by PGK1 Activates PDHK1

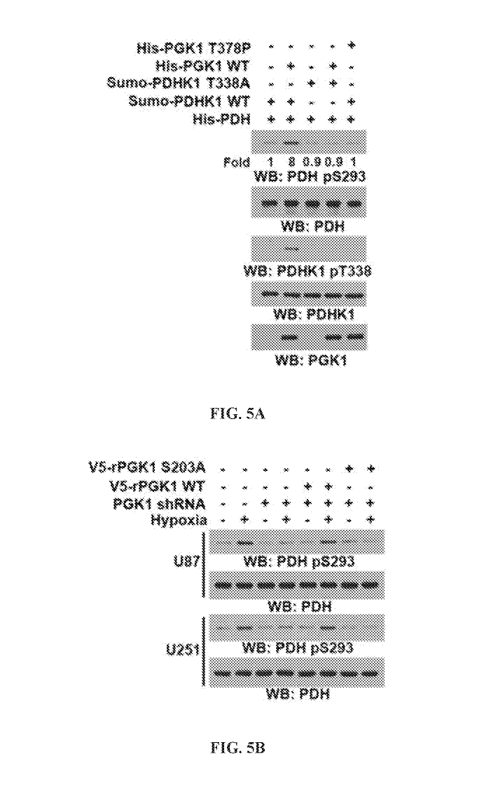

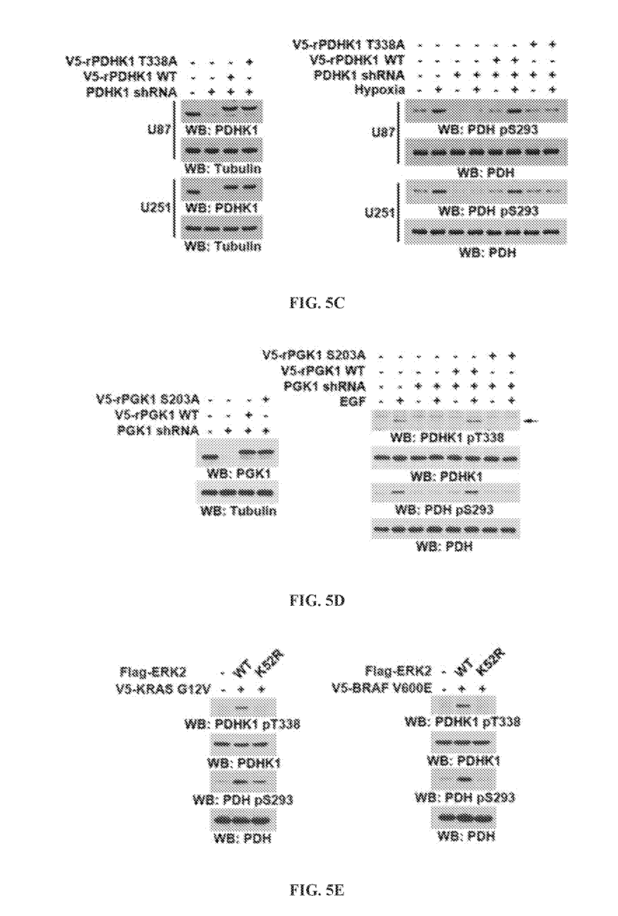

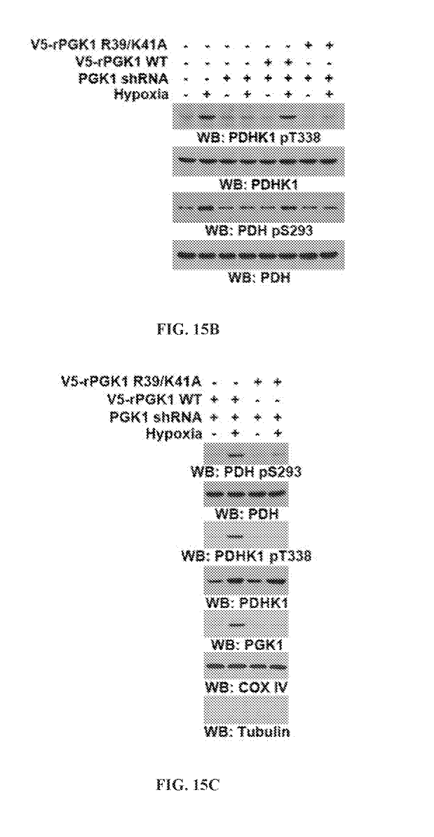

To determine whether PGK1 regulates PDHK1 activity by phosphorylation, the effect of PGK1 on PDHK1-phosphorylated PDH was examined by performing an in vitro protein kinase assay. Mixing bacterially purified His-PDH with purified WT PDHK1 or PDHK1 T338A in the absence or presence of WT PGK1 or the PGK1 T378P mutant and ATP showed that PDHK1-dependent PDH phosphorylation at Ser293 was significantly enhanced by WT PGK1 but not PGK1 T378P (FIG. 5A). In contrast, PDHK1 T338A, whose basal PDH phosphorylation activity was the same as the WT counterpart, did not exhibit PGK1-enhanced PDH phosphorylation (FIG. 5A). These in vitro results were validated in U87 and U251 cells, which showed that expression of PGK1 shRNA blocked hypoxia-induced PDH phosphorylation and that this defect in phosphorylation was rescued by reconstituted expression of WT rPGK1 but not rPGK1 S203A (FIG. 5B), rPGK1 T378P (FIG. 15A), or rPGK1 R39/K41A (FIG. 15B) that had comparable glycolytic activity to its WT counterpart (FIG. 14N). Notably, cells expressing rPGK1 R39/K41A, which does not translocate into mitochondria, did not exhibit increased PDH phosphorylation with long-term hypoxia stimulation, which dramatically enhanced PDHK1 expression (FIG. 15C). These results indicated that PDHK1 requires PGK1-dependent phosphorylation for its full activation.

To further examine the effect of PDHK1 phosphorylation by PGK1 in the regulation of PDH, PDHK1 was depleted from U87 and U251 cells and their expression reconstituted with WT rPDHK1 or rPDHK1 T338A (FIG. 5C, left panel). PDHK1 depletion blocked hypoxia-induced PDH phosphorylation, which was rescued by reconstituted expression of WT rPDHK1 but not rPDHK1 T338A (FIG. 5C, right panel). In addition, EGF treatment (FIG. 5D) or expression of K-Ras G12V and B-Raf V600E (FIG. 5E) resulted in enhanced phosphorylation of PDHK1 T338 and PDH S293, which was blocked by either reconstituted expression of rPGK1 S203A (FIG. 5D) or expression of kinase-dead ERK K52R (FIG. 5E). These results indicated that hypoxia, activation of EGFR, and expression of K-Ras G12V and B-Raf V600E all result in PGK1-mediated phosphorylation of PDHK1, which enhanced PDHK1 activity toward PDH phosphorylation.

### Example 6—PGK1-Mediated PDHK1 Phosphorylation Inhibits Mitochondrial Pyruvate Metabolism and Promotes Glycolysis and Glutaminolysis-Driven Lipid Synthesis

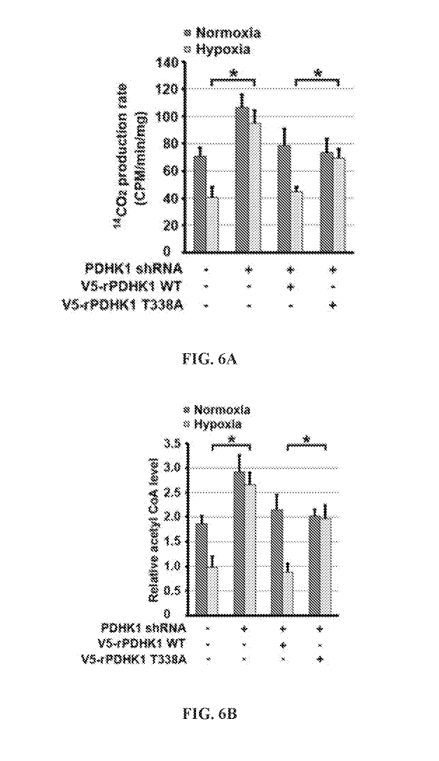

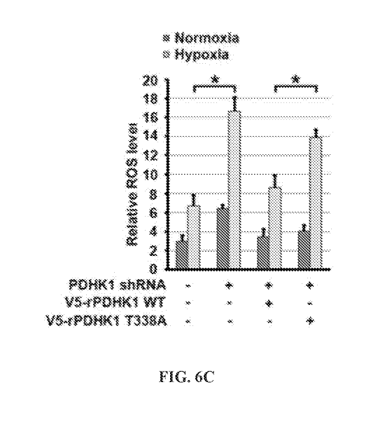

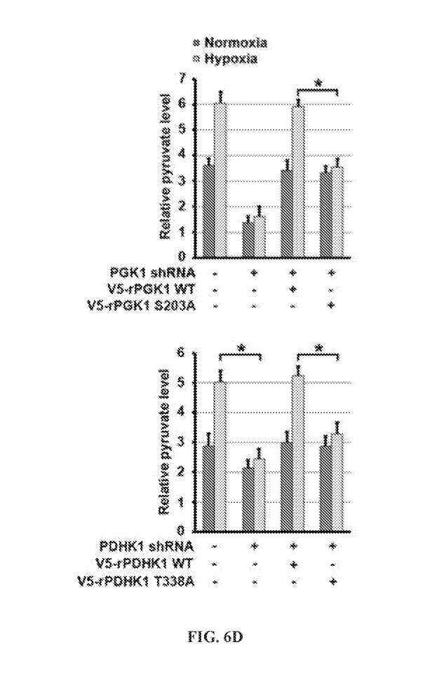

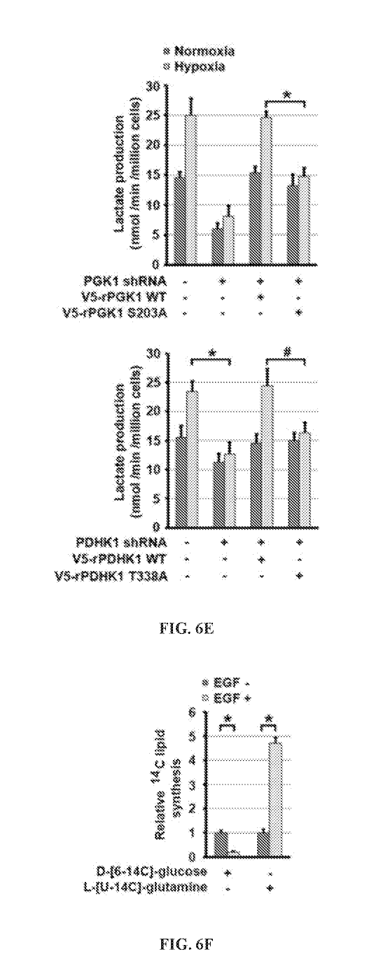

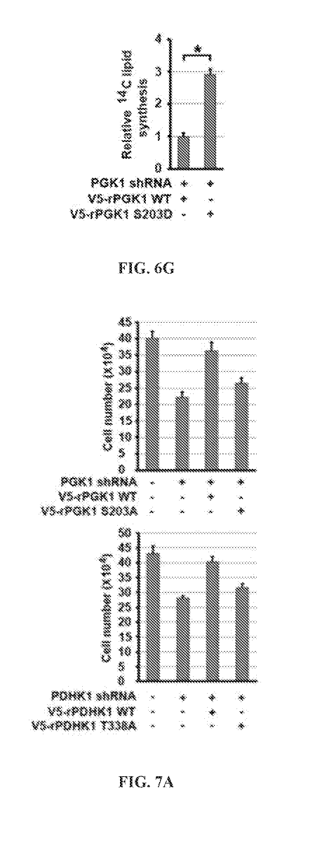

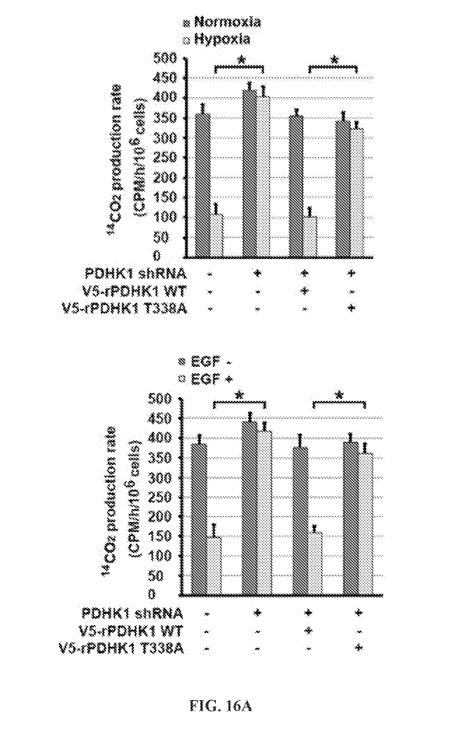

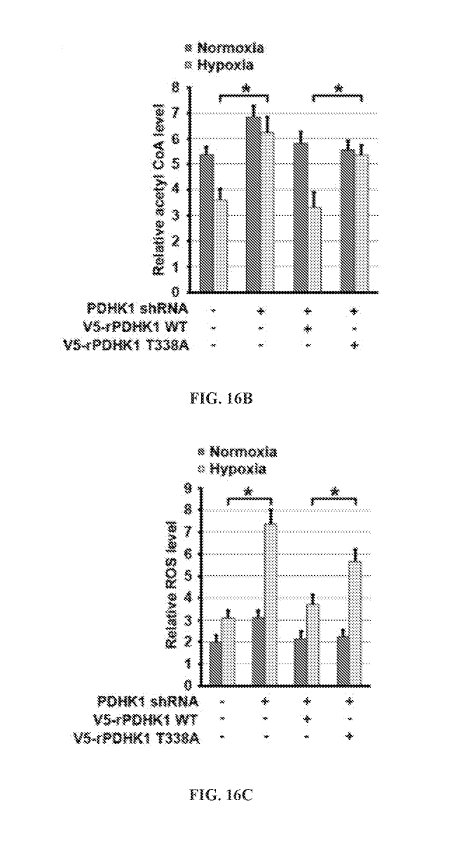

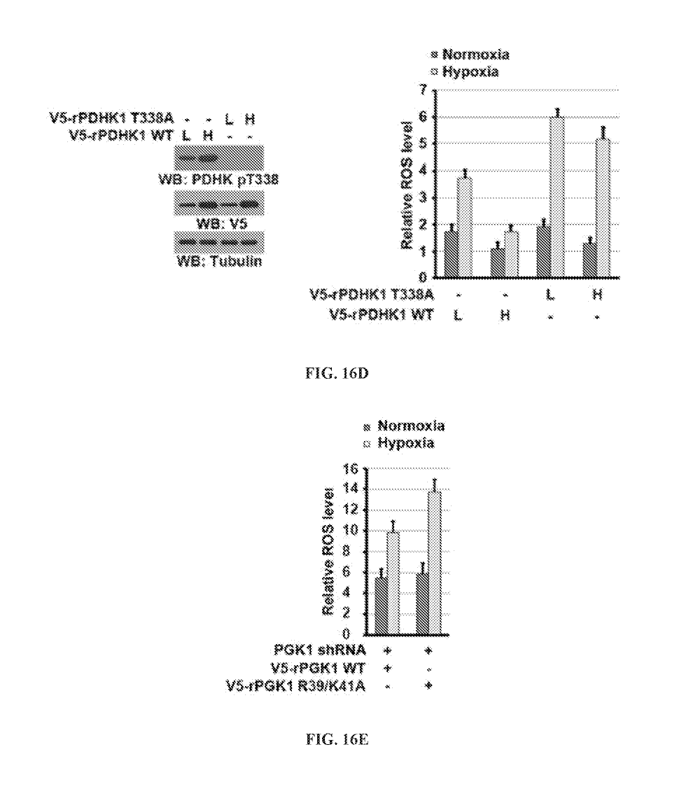

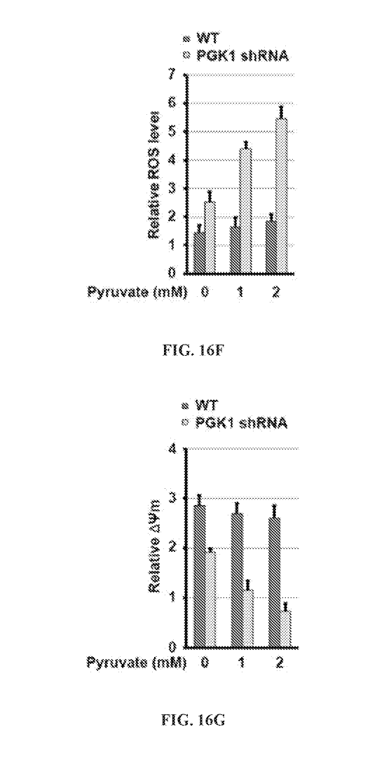

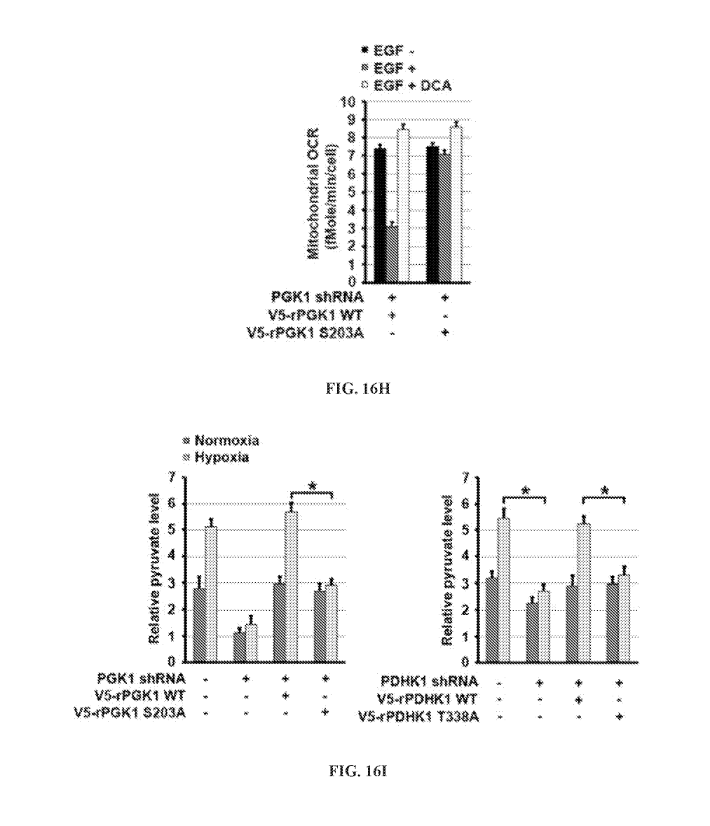

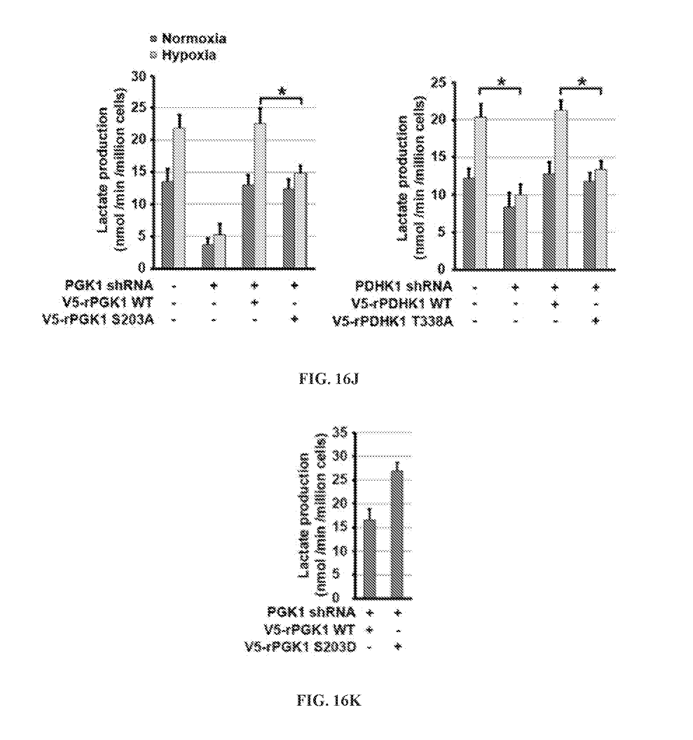

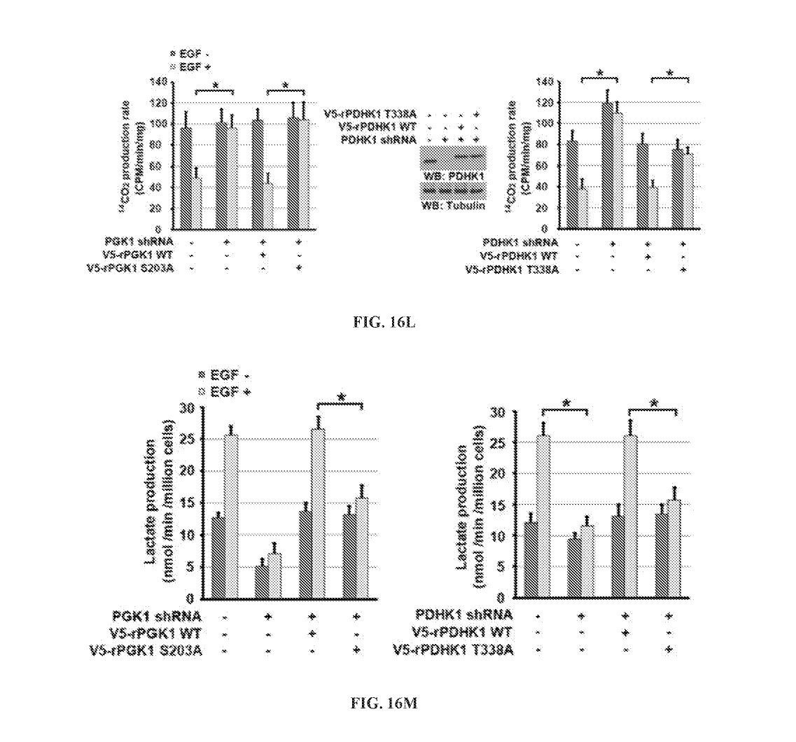

PDHK1 phosphorylates PDH and inhibits its activity (Holness and Sugden, 2003; Kim et al., 2006). To determine the role of PGK1-dependent PDHK1 phosphorylation in regulating mitochondrial function, ¹⁴C-labeled pyruvate was mixed with isolated mitochondria from hypoxia-stimulated U87 cells with or without PDHK1 depletion and reconstituted expression of WT rPDHK1 or rPDHK1 T338A (see FIG. 5C). PDHK1 depletion, acting similarly to rPGK1 S203A expression (FIG. 4A), significantly enhanced the conversion rate of ¹⁴C-labeled pyruvate to ¹⁴C-labeled CO₂ and counteracted the suppression induced by hypoxia (FIG. 6A). These effects were abrogated by reconstituted expression of WT rPDHK1. In contrast, rPDHK1 T338A expression was unable to suppress hypoxia-induced PDH-dependent pyruvate metabolism (FIG. 6A). Similar results were also obtained with hypoxia- or EGF-stimulated U87 cells incubated with [1-¹⁴C]-pyruvate (FIG. 16A). In line with this finding, production of acetyl-CoA levels in mitochondria of U87 cells (FIG. 6B) and U251 cells (FIG. 16B) was suppressed by hypoxia, and this suppression was abrogated by depletion of PDHK1 and restored by reconstituted expression of WT rPDHK1, but not rPDHK1 T338A. Notably, hypoxic stimulation of U87 (FIG. 6C) and U251 cells (FIG. 16C) for 24 h enhanced ROS production, which was further increased by PDHK1 depletion. Reconstituted expression of WT rPDHK1 greatly suppressed ROS production compared to expression of rPDHK1 T338A (FIGS. 6C and 16C). In addition, increased expression of WT rPDHK1, which resulted in an increase in PDHK1 T338 phosphorylation (FIG. 16D, left panel), led to dramatic increase in suppression of ROS production under hypoxic conditions. In contrast, increased expression of rPDHK1 T338A only had limited effects on hypoxia-induced ROS production (FIG. 16D, right panel), suggesting a critical role of PDHK1 T338 phosphorylation in mitochondrial functions of PDHK1. Consistent with this finding, rPGK1 R39/K41A expression enhanced ROS production compared to expression of WT rPGK1 (FIG. 16E). Furthermore, PGK1 depletion induced higher ROS production (FIG. 16F) and mitochondrial membrane potential inhibition (FIG. 16G), which was further enhanced by the addition of exogenous pyruvate. In line with these findings, EGF treatment suppressed the oxygen consumption rate (OCR), and this effect was alleviated by treatment of the cells with dichloroacetate (DCA) PDHK inhibitor or reconstituted expression of PGK1 S203A (FIG. 16H). These results indicate that mitochondrial PGK1-mediated PDHK1 phosphorylation is instrumental in suppressing PDH activity-dependent pyruvate metabolism and hypoxia- or EGF-induced mitochondrial ROS production and respiration.

Consistent with the notion that hypoxia enhances glycolysis (Kim et al., 2006; Papndreou et al., 2006), increased levels of cytosolic pyruvate were detected (FIGS. 6D and 16I) and production of lactate (FIGS. 6E and 16J) in U87 (FIGS. 5D-E) and U251 (FIGS. 16I-J) cells with short term-hypoxic stimulation, which did not alter the expression level of PGK1 and PDHK1 expression (FIGS. 1D and 4G). Notably, this increase was blocked by depletion of PGK1 (left panels) or PDHK1 (right panels), which was completely restored by reconstituted expression of WT rPGK1 or rPDHK1 but not rPGK1 S203A or rPDHK1 T338A, respectively. In addition, reconstituted expression of rPGK1 S203D enhanced lactate production in contrast to reconstituted expression of WT rPGK1 (FIG. 16K).

EGFR activation results in PGK1-mediated phosphorylation and activation of PDHK1 and subsequent PDH phosphorylation. As expected, EGF stimulation repressed the conversion of pyruvate to CO₂ (FIG. 16L) and increased lactate production (FIG. 16M); these effects were alleviated by depletion of PGK1 or PDHK1, which can be rescued by reconstituted expression of WT rPGK1 or WT rPDHK1, but not with rPGK1 S203A or rPDHK1 T338A. These results indicate that mitochondrial PGK1-mediated PDHK1 phosphorylation promotes cytosolic glycolysis by attenuation of mitochondrial pyruvate metabolism.

Glycolysis and glutaminolysis produce citrate via the TCA cycle for fatty acid synthesis. Enhanced conversation of cytosolic pyruvate to lactate and inhibition of mitochondrial pyruvate metabolism may affect the dynamics of fatty acid synthesis contributed from glycolysis and glutaminolysis. Consistent with a previous report that EGFR activation activates glutaminase for glutamine metabolism (Thangavelu et al., 2012), FIG. 6F shows that EGF treatment inhibited ¹⁴C-labeled fatty acid synthesis derived from D-[6-¹⁴C] glucose but greatly increased L-[U-¹⁴C] glutamine-derived lipid synthesis. Importantly, glutaminolysis-promoted lipid synthesis was significantly enhanced by reconstituted expression of rPGK1 S203D compared to the reexpression of WT PGK1 (FIG. 6G). These results strongly suggest that EGFR activation attenuates glycolysis-derived fatty acid synthesis by inhibition of mitochondrial pyruvate metabolism and enhances glutaminolysis-promoted lipid synthesis.

### Example 7—Mitochondrial PGK1-Dependent PDHK1 Phosphorylation Promotes Cell Proliferation and Brain Tumorigenesis and Indicates a Poor Prognosis in GBM Patients

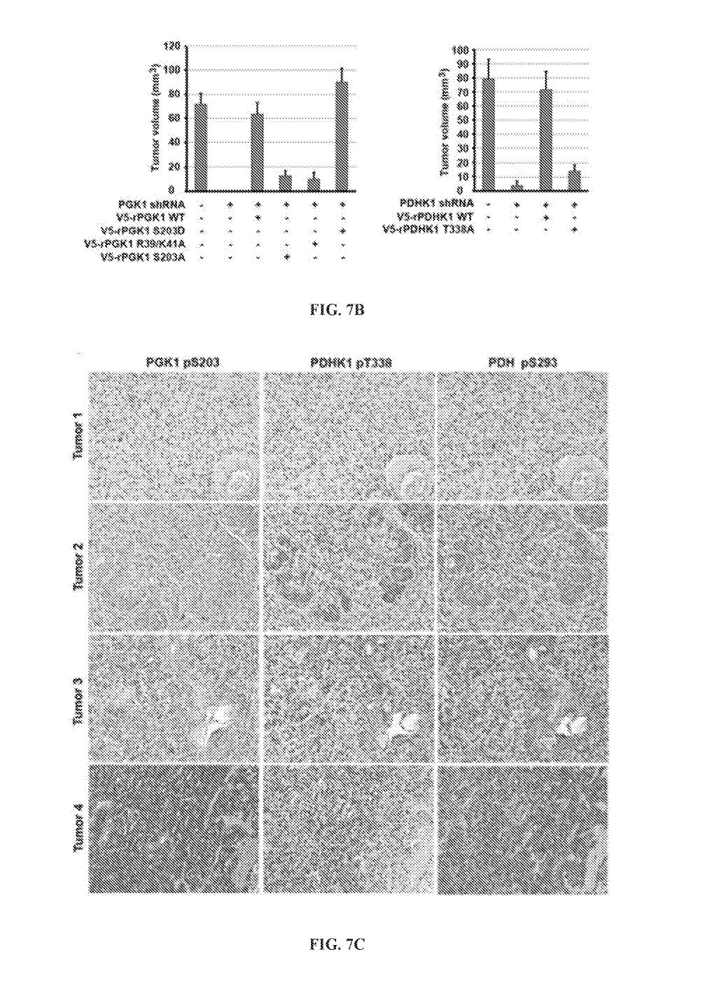

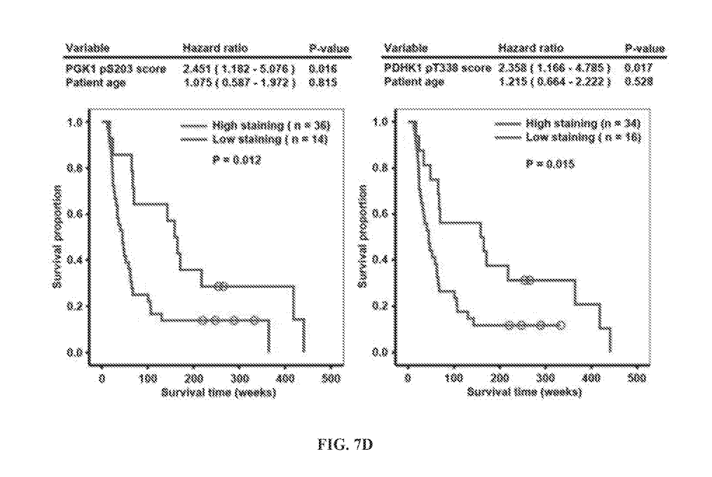

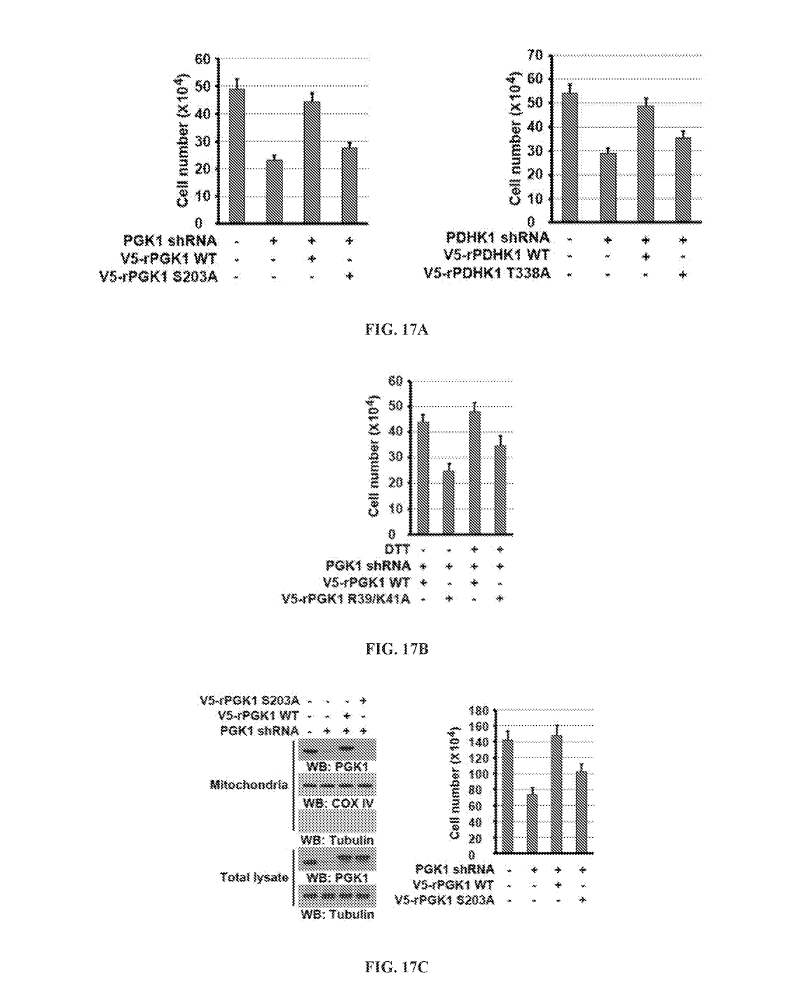

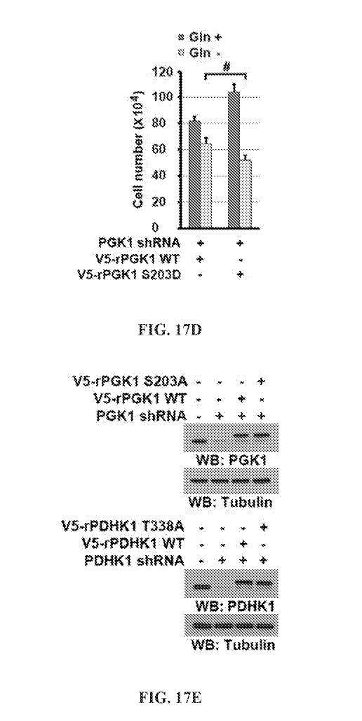

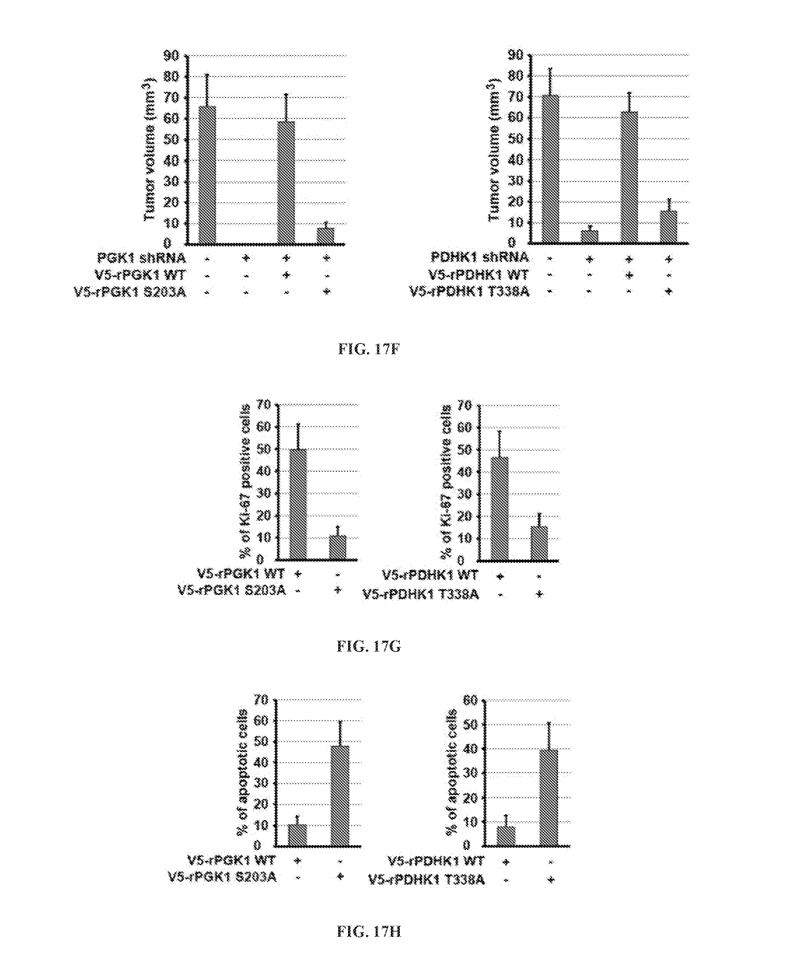

Mitochondrial PGK1-regulated cell metabolism and ROS production likely regulates cell proliferation. As expected, depletion of PGK1 and PDHK1 in U87 and U251 cells, which were in culture for four days under hypoxic conditions, inhibited proliferation (FIGS. 7A and 17A). Reconstituted expression of WT rPGK1 or rPDHK1 restored cell proliferation. In contrast, reconstituted expression of rPGK1 S203A (which maintained cytosolic but not mitochondrial functions) and rPDHK1 T338A (which had basal but not PGK1-enhanced activity) resulted in only partial rescue of these deleterious effects on cells. In line with results from a previous publication (Anastasiou et al. 2011), DTT treatment, which reduced the hypoxia-induced ROS production in mitochondria, partially rescued the hypoxia-induced growth suppression of the U87 cells with reconstituted expression of WT PGK1 or PGK1 R39/K41A mutant (FIG. 17B). Under normoxic conditions, depletion of PGK1 in U87 cells expressing active EGFRvIII mutant inhibited cell proliferation, which was rescued by reconstituted expression of WT rPGK1, but not rPGK1 S203A (FIG. 17C). In contrast, enhanced cell proliferation was observed from the cells expressing rPGK1 S203D, which also rendered the cells more sensitive to glutamine deprivation-induced cell proliferation inhibition (FIG. 17D). These results indicate that mitochondrial PGK1-dependent PDHK1 phosphorylation promotes cell proliferation under both hypoxic and normoxic conditions.

To determine the possible mitochondrial function of PGK1 in brain tumor development, U87 or GSC11 human primary GBM cells were injected intracranially (FIG. 17E) with or without depleted PGK1 or PDHK1 and reconstituted expression of their WT counterparts, rPGK1 S203A, rPGK1 S203D, rPGK1 R39/K41A, or rPDHK1 T338A into athymic nude mice. Dissection of the brains revealed tumor growth in all of the animals injected with U87 cells (FIG. 7B) or GSC11 cells (FIG. 17F). In contrast, no tumor growth or much smaller tumors were detected in the brains of mice injected with the cells with depleted PGK1 or PDHK1, respectively. Reconstituted expression of WT rPGK1 or rPDHK1, but not rPGK1 S203A, rPGK1 R39/K41A, or rPDHK1 T338, in endogenous PGK1- or PDHK1-depleted cells restored tumor growth while rPGK1 S203D-enhanced tumor growth was observed (FIGS. 7B and 17F). Immunohistochemical (IHC) staining revealed strong phosphorylation of PGK1 S203 and PDHK1 T338 in U87 cells with reconstituted expression of their WT counterparts, but not with reconstituted expression of rPGK1 S203A or rPDHK1 T338A. Ki67 staining (FIG. 17G) and analyses with TUNEL assays (FIG. 17H) of tumor tissue revealed rapid cell proliferation and few apoptotic cells with reconstituted expression of WT rPGK1 or rPDHK1, in contrast to slow cell proliferation and more apoptotic cells with reconstituted expression of rPGK1 S203A or rPDHK1 T338A. These results indicate that mitochondrial PGK1-dependent PDHK1 phosphorylation promotes brain tumorigenesis.

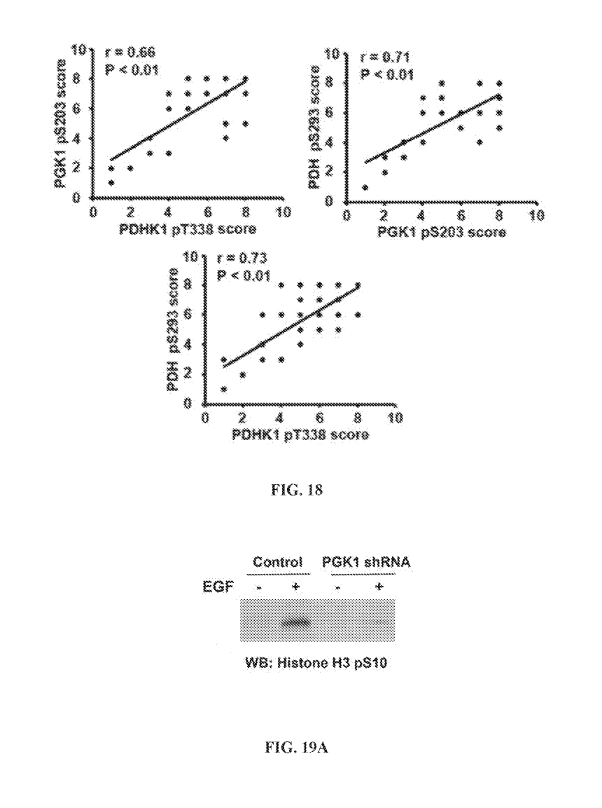

To determine the clinical relevance of the finding that mitochondrial PGK1-dependent PDHK1 phosphorylation regulates PDH activity, IHC analysis was performed with 50 human primary GBM specimens (World Health Organization grade IV) with anti-phospho-PGK1 S203, anti-phospho-PDHK1 T338, and anti-phospho-PDH S293 antibodies. The antibody specificities were validated (Kaplon et al., 2013) by using IHC analyses with specific blocking peptides. As shown in FIG. 6C, the phosphorylation levels of PGK1 S203, PDHK1 T338, and PDH S293 were correlated with each other. Quantification of the staining showed that these correlations were significant (FIG. 18).

The survival duration of the 50 patients, all of whom had received standard adjuvant radiotherapy after surgical resection of GBM followed by treatment with an alkylating agent (temozolomide in most cases), were compared with tumor phosphorylation levels of PGK1 S203 and PDHK1 T338 (low: staining score 0-4; high: staining score 4.1-8). The median survival duration was 201.3 and 192.4 weeks for patients whose tumors had low PGK1 S203 and PDHK1 T338 phosphorylation levels, respectively, and 90.2 and 82.9 weeks for those whose tumors had high phosphorylation levels of PGK1 S203 and PDHK1 T338, respectively. In a Cox multivariate model, the IHC scores of PGK1 S203 and PDHK1 T338 phosphorylation were independent predictors of GBM patient survival after adjustment for patient age, which is a relevant clinical covariate (FIG. 7D). These results support a role for mitochondrial PGK1-dependent PDHK1 phosphorylation in the clinical behavior of human GBM and reveal a correlation among ERK1/2-dependent PGK1 phosphorylation, PGK1-dependent PDHK1 phosphorylation, and the clinical aggressiveness of GBM.

### Example 8—PGK1 Phosphorylates Histone H2, CDC45, and Beclin-1

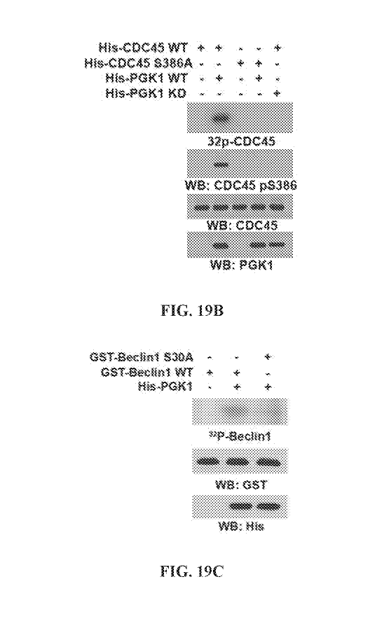

As shown in FIG. 19A, PGK1 phosphorylates histone H3 at Ser10. In addition, expression of PGK1 shRNA in U87 cells blocked EGF-induced phosphorylation of histone H3 at Ser10, which is important for gene transcription and mitosis progression.

CDC45 is an essential protein required for the initiation of DNA replication. Purified wild-type PGK1 but not PGK1 kinase-dead (KD) mutant phosphorylated purified wild-type CDC45, but not CDC45 S386A, in the presence of [f³²P]-ATP (FIG. 19B).

Beclin-1 is involved in initiation of autophagy. Purified PGK1 phosphorylated purified wild-type Beclin-1 in the presence of [f³P]-ATP (FIG. 19C). Beclin-1 S30A mutant was largely resistant to phosphorylation by PGK1.

### Example 9—Autophosphorylation at Y324 of PGK1 Increased PGK1 Glycolytic Enzyme Activity

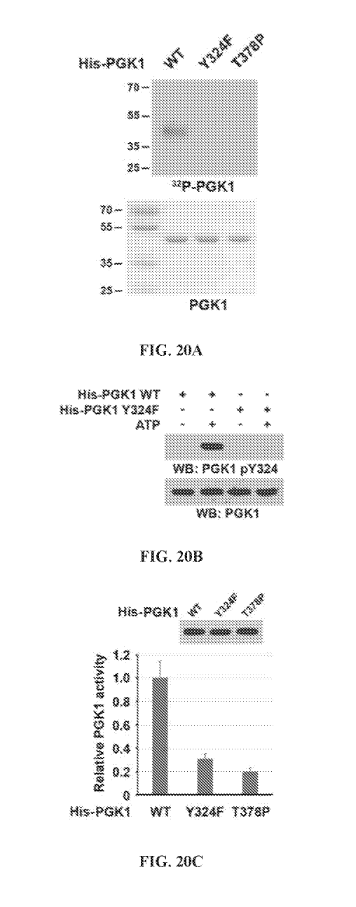

PGK1, a glycolytic enzyme that produces ATP in glycolysis, functions as a protein kinase utilizing ATP to phosphorylate its substrate. In addition to phosphorylating other proteins, PGK1 can undergo autophosphorylation. Mass spectrometry analysis identified tyrosine 324 (Y324) as an autophosphorylation site of PGK1. In vitro protein kinase assays showed that substitution from tyrosine 324 to phenylalanine (Y324F) prevented autophosphorylation (FIGS. 20A-B). A PGK1 enzyme activity assay showed that the glycolytic enzyme activity of the PGK1 Y324F mutant is severely reduced relative to that of the wild-type (WT) enzyme, and comparable to that of PGK1 T378P, an enzyme activity-dead mutant that disrupts binding of ATP (FIG. 20C).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,870,287
U.S. Pat. No. 5,739,169
U.S. Pat. No. 5,760,395
U.S. Pat. No. 5,801,005
U.S. Pat. No. 5,824,311
U.S. Pat. No. 5,830,880
U.S. Pat. No. 5,846,945
U.S. Pat. Publn. 2008/004287
Ahmad et al., Phosphoglycerate kinase 1 as a promoter of metastasis in colon cancer. International journal of oncology, International Journal of Oncology, 43:586-590, 2013.
Ai et al., FLNA and PGK1 are two potential markers for progression in hepatocellular carcinoma. Cellular Physiology and Biochemistry: International Journal of Experimental Cellular Physiology, Biochemistry, and Pharmacology, 27:207-216, 2011.
Anastasiou et al., Inhibition of pyruvate kinase M2 by reactive oxygen species contributes to cellular antioxidant responses, Science, 334:1278-1283, 2011.
Austin-Ward and Villaseca, Revista Medica de Chile, 126(7):838-845, 1998.
Bain et al., The selectivity of protein kinase inhibitors: a further update, Biochem. J., 408:297-315, 2007.
Bernstein and Hol, Crystal structures of substrates and products bound to the phosphoglycerate kinase active site reveal the catalytic mechanism, Biochemistry, 37:4429-4436, 1998.
Brahimi-Horn et al., Hypoxia signalling controls metabolic demand. Current Opinion in Cell Biology, 19:223-229, 2007.
Brown, Control of respiration and ATP synthesis in mammalian mitochondria and cells, The Biochemical Journal, 284:1-13, 1992.
Bukowski et al., Clinical Cancer Res., 4(10):2337-2347, 1998.
Cairns et al., Regulation of cancer cell metabolism, Nature Reviews, Cancer 11:85-95, 2011.
Chacinska et al., Importing mitochondrial proteins: machineries and mechanisms, Cell, 138:628-644, 2009.
Chiarelli et al., Molecular insights on pathogenic effects of mutations causing phosphoglycerate kinase deficiency, PloS ONE, 7:e32065, 2012.
Chow et al., Measurement of MAP kinase activation by flow cytometry using phospho-specific antibodies to MEK and ERK: potential for pharmacodynamic monitoring of signal transduction inhibitors, Cytometry, 46:72-78, 2001.
Christodoulides et al., Microbiology, 144 (Pt 11):3027-3037, 1998.
Davidson et al., J. Immunother., 21(5):389-398, 1998.
Davies et al., Specificity and mechanism of action of some commonly used protein kinase inhibitors, Biochem. J., 341:95-105, 2000.
“Design of Prodrugs”, ed. H. Bundgaard, Elsevier, 1985.
Downey et al., Chemotactic Peptide-induced Activation of MEK-2, the Predominant Isoform in Human Neutrophils: Inhibition by Wortmannin, J. Biol. Chem., 271:21005-21011, 1996.
Duncan et al., Discovery and characterization of a nonphosphorylated cyclic peptide inhibitor of the peptidylprolyl isomerase, Pin1, J. Med. Chem., 54:3854-3865, 2011.
Duan et al., Overexpression of human phosphoglycerate kinase 1 (PGK1) induces a multidrug resistance phenotype, Anticancer Research, 22L1933-1941, 2002.
English and Cobb, Pharmacological inhibitors of MAPK pathways, Trends Pharmacol Sci.,
23:40-45, 2002.
Fang et al., Phosphorylation of beta-catenin by AKT promotes beta-catenin transcriptional activity, The Journal of Biological Chemistry, 282:11221-11229, 2007.
Flockhart et al., NFAT signalling is a novel target of oncogenic BRAF in metastatic melanoma, British Journal of Cancer, 101:1448-1455, 2009.
Gao et al., c-Myc suppression of miR-23a/b enhances mitochondrial glutaminase expression and glutamine metabolism, Nature, 458:762-765, 2009.
Gao et al., Pyruvate kinase M2 regulates gene transcription by acting as a protein kinase, Molecular Cell, 45:598-609, 2012.
Gatenby and Gillies, Why do cancers have high aerobic glycolysis? Nature reviews, Cancer, 4:891-899, 2004.
Gomez-Manzano et al., Delta-24 increases the expression and activity of topoisomerase I and enhances the antiglioma effect of irinotecan, Clin. Cancer Res., 12:556-562, 2006.
Hanibuchi et al., Int. J. Cancer, 78(4):480-485, 1998.
Hellstrand et al., Acta Oncologica, 37(4):347-353, 1998.
Hitosugi et al., Tyrosine phosphorylation of mitochondrial pyruvate dehydrogenase kinase 1 is important for cancer metabolism, Molecular Cell, 44:864-877, 2011.
Hockel and Vaupel, Tumor hypoxia: definitions and current clinical, biologic, and molecular aspects, Journal of the National Cancer Institute, 93:266-276, 2001.
Holness and Sugden, Regulation of pyruvate dehydrogenase complex activity by reversible phosphorylation, Biochemical Society Transactions, 31:1143-1151, 2003.
Hsieh et al., Current prodrug design for drug discovery, Curr. Pharm. Des., 15:2236-2250, 2009.
Hui and Hashimoto, Infection Immun., 66(11):5329-5336, 1998.
Huttunen et al., Prodrugs—from Serendipity to Rational Design, Pharmacological Rev., 63:750-771, 2011.
Hwang et al., Overexpression and elevated serum levels of phosphoglycerate kinase 1 in pancreatic ductal adenocarcinoma, Proteomics, 6:2259-2272, 2006.
Ji et al., EGF-induced ERK activation promotes CK2-mediated disassociation of alpha-Catenin from beta-Catenin and transactivation of beta-Catenin, Molecular Cell, 36:547-559, 2009.
Jiang et al., PKM2 Regulates Chromosome Segregation and Mitosis Progression of Tumor Cells, Molecular Cell, 53:75-87, 2014.
Kaplon et al., A key role for mitochondrial gatekeeper pyruvate dehydrogenase in oncogene-induced senescence, Nature, 498:109-112, 2013.
Kim et al., HIF-1-mediated expression of pyruvate dehydrogenase kinase: a metabolic switch required for cellular adaptation to hypoxia, Cell Metabolism, 3:177-185, 2006.
Klein et al., The effects of a novel MEK inhibitor PD184161 on MEK-ERK signaling and growth in human liver cancer, Neoplasia, 8:1-8, 2006.
Koppenol et al., Otto Warburg's contributions to current concepts of cancer metabolism, Nature Reviews, Cancer, 11:325-337, 2011.
Kronblad et al., ERK1/2 inhibition increases antiestrogen treatment efficacy by interfering with hypoxia-induced downregulation of ERalpha: a combination therapy potentially targeting hypoxic and dormant tumor cells, Oncogene, 24:6835-6841, 2005.
Lowery et al., Proteomic screen defines the Polo-box domain interactome and identifies Rock2 as a Plk1 substrate, The EMBO Journal, 26:2262-2273, 2007.
Lu and Xu, ERK1/2 MAP kinases in cell survival and apoptosis, IUBMB Life, 58:621-631, 2006.
Lu and Zhou, The prolyl isomerase PIN1: a pivotal new twist in phosphorylation signalling and disease, Nature Reviews, Molecular Cell Biology, 8:904-916, 2007.
Lu et al., Activation of protein kinase C triggers its ubiquitination and degradation, Molecular and Cellular Biology, 18:839-845, 1998.
Marin-Hernandez et al., HIF-lalpha modulates energy metabolism in cancer cells by inducing over-expression of specific glycolytic isoforms, Mini Reviews in Medicinal Chemistry,
9:1084-1101, 2009.
Mattingly et al., The mitogen-activated protein kinase/extracellular signal-regulated kinase kinase inhibitor PD184352 (CI-1040) selectively induces apoptosis in malignant schwannoma cell lines, J. Pharmacol. Exp. Ther., 316:456-465, 2006.
Michelson et al., Structure of the human phosphoglycerate kinase gene and the intron-mediated evolution and dispersal of the nucleotide-binding domain, Proceedings of the National Academy of Sciences USA, 82:6965-6969 1985.
Mori et al., A dual inhibitor against prolyl isomerase Pin1 and cyclophilin discovered by a novel real-time fluorescence detection method, Biochem. Biophys, Res. Commun., 406:439-443, 2011.
Papandreou et al., HIF-1 mediates adaptation to hypoxia by actively down-regulating mitochondrial oxygen consumption, Cell Metabolism, 3:187-197, 2006.
Pezzato et al., Ca²⁺-independent effects of spermine on pyruvate dehydrogenase complex activity in energized rat liver mitochondria incubated in the absence of exogenous Ca²⁺ and Mg²⁺, Amino Acids, 36:449-456, 2009.
Qin et al., Proc. Natl. Acad. Sci. USA, 95(24):14411-14416, 1998.
Riley et al., Chromatin structure of active and inactive human X-linked phosphoglycerate kinase gene, Somatic Cell and Molecular Genetics, 12:73-80, 1986.
Rinehart et al., Multicenter phase II study of the oral MEK inhibitor, CI-1040, in patients with advanced non-small-cell lung, breast, colon, and pancreatic cancer, J. Clin. Oncol., 22:4456-4462, 2004.
Roche and Hiromasa, Pyruvate dehydrogenase kinase regulatory mechanisms and inhibition in treating diabetes, heart ischemia, and cancer, Cellular and Molecular Life Sciences: CMLS, 64:830-849, 2007.
Semenza, Hypoxia-inducible factor 1 and cancer pathogenesis, IUBMB Life, 60:591-597, 2008.
Semenza, HIF-1: upstream and downstream of cancer metabolism, Current Opinion in Genetics and Development, 20:51-56, 2010.
She et al., Direct regulation of complex I by mitochondrial MEF2D is disrupted in a mouse model of Parkinson disease and in human patients, The Journal of Clinical Investigation, 121:930-940, 2011.
Tani et al., Molecular cloning and structure of an autosomal processed gene for human phosphoglycerate kinase, Gene, 35:11-18, 1985.
Taus et al., Universal and confident phosphorylation site localization using phosphoRS, Journal of Proteome Research, 10:5354-5362, 2011.
Thangavelu et al., Structural basis for the allosteric inhibitory mechanism of human kidney-type glutaminase (KGA) and its regulation by Raf-Mek-Erk signaling in cancer cell metabolism, Proceedings of the National Academy of Sciences USA, 109:7705-7710, 2012.
van der Geer and Hunter, Phosphopeptide mapping and phosphoamino acid analysis by electrophoresis and chromatography on thin-layer cellulose plates, Electrophoresis, 15:544-554, 1994.
Vander Heiden et al., Understanding the Warburg effect: the metabolic requirements of cell proliferation, Science, 324:1029-1033, 2009.
Wykosky et al., Therapeutic targeting of epidermal growth factor receptor in human cancer: successes and limitations, Chinese Journal of Cancer, 30:5-12, 2011.
Xia et al., c-Jun down-regulation by HDAC3-dependent transcriptional repression promotes osmotic stress-induced cell apoptosis, Molecular Cell, 25:219-232, 2007.
Xu et al., Reduced-Amide Inhibitor of Pin1 Binds in a Conformation Resembling a Twisted-Amide Transition State, Biochemistry, 50:9545-9550, 2011.
Xu et al., Cyclohexyl Ketone Inhibitors of Pin1 Dock in a Trans-Diaxial Cyclohexane Conformation, PLoS ONE, 7:e44226, 2012.
Yaffe et al., Sequence-specific and phosphorylation-dependent proline isomerization: a potential mitotic regulatory mechanism, Science, 278:1957-1960, 1997.
Yan et al. Over-expression of cofilin-1 and phosphoglycerate kinase 1 in astrocytomas involved in pathogenesis of radioresistance, CNS Neuroscience and Therapeutics, 18:729-736, 2012.
Yang et al., ERK1/2-dependent phosphorylation and nuclear translocation of PKM2 promotes the Warburg effect, Nature Cell Biology, 14:1295-1304, 2012.
Yang et al., PKM2 phosphorylates histone H3 and promotes gene transcription and tumorigenesis, Cell, 150:685-696, 2012.
Yoon et al., Inhibition of Plk1 and Pin1 by 5′-nitro-indirubinoxime suppresses human lung cancer cells, Cancer Lett., 316:97-104, 2012.
Yun et al., Glucose deprivation contributes to the development of KRAS pathway mutations in tumor cells, Science, 325:1555-1559, 2009.
Zhang et al., Synthesis and structure-activity relationships of 3-cyano-4-(phenoxyanilino)quinolines as MEK (MAPKK) inhibitors, Bioorg. Med. Chem. Lett., 10:2825-2828, 2000.
Zhang et al., Proteomic study reveals that proteins involved in metabolic and detoxification pathways are highly expressed in HER-2/neu-positive breast cancer, Molecular & Cellular Proteomics: MCP, 4:1686-1696, 2005.
Zheng et al., FAK phosphorylation by ERK primes ras-induced tyrosine dephosphorylation of FAK mediated by PIN1 and PTP-PEST, Molecular Cell, 35:11-25, (2009).
Zieker et al., Phosphoglycerate kinase 1 a promoting enzyme for peritoneal dissemination in gastric cancer. International journal of cancer, Journal International du Cancer, 126:1513-1520, 2010.

What is claimed is:

1. A composition for use in treating a patient having a cancer determined to comprise: an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level, the composition comprising a PGK1 inhibitor, a MEK/ERK inhibitor, a EGFR inhibitor, or a PIN1 inhibitor.

2. The composition of claim 1, wherein the cancer is an oncogenic EGFR, an oncogenic K-Ras, or oncogenic B-Raf positive cancer.

3. The composition of claim 1, wherein the cancer is a glioma.

4. The composition of claim 1, wherein the cancer is a oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumor, thyroid cancer, parathyroid cancer, pituitary tumor, adrenal gland tumor, osteogenic sarcoma tumors, neuroendocrine tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

5. The composition of claim 1, comprising a PGK1 inhibitor.

6. The composition of claim 5, wherein the PGK1 inhibitor is a small molecule PGK1 inhibitor.

7. The composition of claim 6, wherein the small molecule PGK1 inhibitor selectively inhibits the kinase activity of PGK1.

8. The composition of claim 5, wherein the PGK1 inhibitor comprises an inhibitory polynucleotide complementary to all or part of a PGK1 gene.

9. The composition of claim 8, wherein the inhibitory polynucleotide is a siRNA.

10. The composition of claim 1, further comprising at least a second therapeutic.

11. The composition of claim 10, wherein the second therapy is a MEK/ERK inhibitor therapy.

12. The composition of claim 1, comprising a MEK/ERK inhibitor.

13. The composition of claim 12, wherein the MEK/ERK inhibitor is U0126, AZD6244, PD98059, GSK1120212, GDC-0973, RDEA119, PD18416, CI1040 or FR180204.

14. The composition of claim 1, comprising an EGFR inhibitor.

15. The composition of claim 14, wherein the EGFR inhibitor is AG1478.

16. The composition of claim 1, comprising a PIN1 inhibitor.

17. A method for treating a patient having a cancer comprising:
(a) selecting a patient whose cancer cells have been determined to comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level; and
(b) treating the patient with a PGK1 inhibitor therapy, a MEK/ERK inhibitor therapy, a EGFR inhibitor therapy, or a PIN1 inhibitor therapy.

18. An in vitro method of selecting a patient having a cancer for a PGK1 inhibitor therapy, a MEK/ERK inhibitor therapy, a EGFR inhibitor therapy, or a PIN1 inhibitor therapy comprising determining whether cancer cell of the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level, wherein if the patient comprises an elevated level then the patient is selected for a PGK1 inhibitor therapy, a MEK/ERK inhibitor therapy, a EGFR inhibitor therapy, or a PIN1 inhibitor therapy.

19. An in vitro method of selecting a patient having a cancer for a PGK1 inhibitor therapy, a MEK/ERK inhibitor therapy, a EGFR inhibitor therapy, or a PIN1 inhibitor therapy comprising (a) determining whether cancer cell of the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level, and (b) selecting a patient for a PGK1 inhibitor therapy, a MEK/ERK inhibitor therapy, a EGFR inhibitor therapy, or a PIN1 inhibitor therapy if cancer cells of the patient comprise an elevated level.

20. A method of predicting a response to a PGK1 inhibitor therapy, a MEK/ERK inhibitor therapy, a EGFR inhibitor therapy, or a PIN1 inhibitor therapy in a patient having cancer comprising determining whether cancer cells of the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level, wherein if the cancer cells comprise an elevated level, then the patient is predicted to have a favorable response to a PGK1 inhibitor therapy, a MEK/ERK inhibitor therapy, a EGFR inhibitor therapy, or a PIN1 inhibitor therapy.

21. A method of predicting a response to a PGK1 inhibitor therapy, a MEK/ERK inhibitor therapy, a EGFR inhibitor therapy, or a PIN1 inhibitor therapy in a patient having cancer comprising (a) determining whether cancer cells of the patient comprise: an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level, and (b) identifying the patient as predicted to have a favorable response to a PGK1 inhibitor therapy, a MEK/ERK inhibitor therapy, a EGFR inhibitor therapy, or a PIN1 inhibitor therapy if cancer cells from the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level; or identifying the patient as not predicted to have a favorable response to a PGK1 inhibitor therapy, a MEK/ERK inhibitor therapy, a EGFR inhibitor therapy, or a PIN1 inhibitor therapy if cancer cells from the patient do not comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation; or an elevated level of Beclin-1 S30 phosphorylation compared to a reference level.

22. The method of claim 21, further comprising reporting whether cancer cells of the patient comprise an elevated level.

23. The method of claim 22, wherein the reporting comprises providing a written or electronic report.

24. The method of claim 21, further comprising reporting whether that patient was identified as predicted or not predicted to have a favorable response to a PGK1 inhibitor therapy, a MEK/ERK inhibitor therapy, a EGFR inhibitor therapy, or a PIN1 inhibitor therapy.

25. The method of claim 24, wherein the reporting comprises providing a written or electronic report.

26. The method of claim 21, wherein the determining comprises use of a phosphorylation specific antibody.

27. The method of claim 21, wherein the determining comprises performing an ELISA, an immunoassay, a radioimmunoassay (RIA), immunohistochemistry, an immunoradiometric assay, a fluoroimmunoassay, a chemiluminescent assay, a bioluminescent assay, a gel electrophoresis, a Western blot analysis, a southern blot, flow cytometry, in situ hybridization, positron emission tomography (PET), single photon emission computed tomography (SPECT) imaging) or a microscopic assay.

28. The method of claim 21, wherein a favorable response comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, increased patient survival or an increase in the sensitivity of the tumor to an anticancer therapy.

29. The method of claim 21, wherein the reference level is a level from a non-cancer cell.

30. The method of claim 21, wherein the reference level is a level from an early stage or low-grade cancer cell.

31. A method of determining a prognosis in a patient having a cancer comprising determining whether cancer cells of the patient comprise an elevated level of PGK1 S203 phosphorylation or an elevated level of PDHK1 T338 phosphorylation compared to a reference level, wherein if the cancer cells comprise an elevated level of PGK1 S203 phosphorylation or an elevated level of PDHK1 T338 phosphorylation, then the patient is predicted to have an aggressive cancer.

32. An in vitro method of determining a prognosis in a patient having a cancer comprising: (a) determining whether cancer cells of the patient comprise an elevated level of PGK1 S203 phosphorylation; an elevated level of PGK1 Y324 phosphorylation; an elevated level of PDHK1 T338 phosphorylation; an elevated level of PDH S293 phosphorylation; an elevated level of CDC45 S386 phosphorylation; an elevated level of histone H3 S10 phosphorylation compared to a reference level; and (b) identifying the patient as predicted to have an aggressive cancer, if cancer cells from the patient comprise the elevated level or identifying the patient as not predicted to have an aggressive cancer, if cancer cells from the patient do not comprise the elevated level.

33. The method of claim 32, further comprising administering one or more anticancer therapy to the patient if the patient is predicted to have an aggressive cancer.

34. The method of claim 32, wherein the reference level is a level from a non-cancer cell.

35. The method of claim 32, wherein the reference level is a level from an early stage or low grade cancer cell.

36. The method of claim 32, further comprising reporting whether cancer cells of the patient comprise an elevated level.

37. The method of claim 36, wherein the reporting comprises providing a written or electronic report.

38. The method of claim 32, further comprising reporting whether the patient was identified as predicted or not predicted to have an aggressive cancer.

39. A method for screening candidate PGK1 inhibitors or anti-cancer agents comprising determining the binding of PGK1 to PDHK1 and/or the phosphorylation of PDHK1 by PGK1 in the presence or absence of an agent, wherein an agent that disrupts binding of PGK1 to PDHK1 and/or disrupts phosphorylation of PDHK1 by PGK1 is a candidate PGK1 inhibitor or anti-cancer agent.

40. A method for screening candidate PGK1 inhibitors or anti-cancer agents comprising:
(a) determining the binding of PGK1 to PDHK1 and/or the phosphorylation of PDHK1 by PGK1 in the presence or absence of an agent; and
(b) selecting a candidate PGK1 inhibitor or anti-cancer agent based on the agent disrupting the binding of PGK1 to PDHK1 and/or the phosphorylation of PDHK1 by PGK1.

41. The method of claim 40, wherein the agent is a small molecule.

42. The method of claim 41, further defined as a cell-free method.

43. An in vitro method of predicting the severity of a cancer in a patient comprising:
(a) determining a level of PGK1 activity, a level of PGK1 S203 phosphorylation, a level of PGK1 Y324 phosphorylation, or a level of PGK1 mitochondrial localization in a patient sample; and
(b) predicting the severity of a cancer in the subject based on the level of PGK1 activity, a level of PGK1 S203 phosphorylation, a level of PGK1 Y324 phosphorylation, or a level of PGK1 mitochondrial localization, wherein an elevated level of PGK1 activity, PGK1 S203 phosphorylation, PGK1 Y324 phosphorylation, or PGK1 mitochondrial localization relative to a reference level indicates a more severe cancer.

44. The method of claim 43, wherein determining a level of PGK1 activity comprises determining a level of PDHK1 T338 phosphorylation.

45. The method of claim 43, wherein determining comprises determining a level of PGK1 S203 phosphorylation.

46. The method of claim 43, wherein determining comprises determining a level of PGK1 Y324 phosphorylation.

47. The method of claim 43, wherein determining comprises determining a level of PGK1 mitochondrial localization.

48. The method of claim 44, wherein determining the level of PDHK1 T338 phosphorylation comprises contacting the sample with a phosphorylation specific antibody.

49. The method of claim 45, wherein determining the level of PGK1 S203 phosphorylation comprises contacting the sample with a phosphorylation specific antibody.

50. The method of claim 46, wherein determining the level of PGK1 Y324 phosphorylation comprises contacting the sample with a phosphorylation specific antibody.

51. The method of claim 43, wherein determining the level of PGK1 activity, PGK1 S203 phosphorylation, PGK1 Y324 phosphorylation, or PGK1 mitochondrial localization comprises performing an ELISA, an immunoassay, a radioimmunoassay (RIA), immunohistochemistry, an immunoradiometric assay, a fluoroimmunoassay, a chemiluminescent assay, a bioluminescent assay, a gel electrophoresis, a Western blot analysis, a southern blot, flow cytometry, in situ hybridization, positron emission tomography (PET), single photon emission computed tomography (SPECT) imaging) or a microscopic assay.

52. The method of claim 43, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumor, thyroid cancer, parathyroid cancer, pituitary tumor, adrenal gland tumor, osteogenic sarcoma tumor, neuroendocrine tumor, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

53. The method of claim 43, wherein the sample is a tumor biopsy sample.


SEQUENCE LISTING (10 sequences)

SEQ ID NO: 1 - protein, 14 aa, Artificial Sequence
  Synthetic polypeptide
HHHHHHLESPERNA

SEQ ID NO: 2 - protein, 14 aa, Artificial Sequence
  Synthetic polypeptide
HHHHHHLEDPERNA

SEQ ID NO: 3 - DNA, 21 bp, Artificial Sequence
  Synthetic oligonucleotide
gcttctaacaccggaggtctt

SEQ ID NO: 4 - DNA, 21 bp, Artificial Sequence
  Synthetic oligonucleotide
ggatgtctatgtcaatgatgc

SEQ ID NO: 5 - DNA, 19 bp, Artificial Sequence
  Synthetic oligonucleotide
ccgaactagaacttgaaga

SEQ ID NO: 6 - protein, 11 aa, Artificial Sequence
  Synthetic polypeptide
DKYSLEPVAVE

SEQ ID NO: 7 - protein, 11 aa, Artificial Sequence
  Synthetic polypeptide
HRAHSSMVGVN

SEQ ID NO: 8 - protein, 11 aa, Artificial Sequence
  Synthetic polypeptide
AEKNGVKITLP

SEQ ID NO: 9 - protein, 6 aa, Artificial Sequence
  Synthetic polypeptide
QRIKAA

SEQ ID NO: 10 - protein, 13 aa, Artificial Sequence
  Synthetic polypeptide
LFNYMYSTAPRPR